(19) United States
(12) Patent Application Publication
(10) Pub. No.: US 2022/0020450 A1
(43) Pub. Date: Jan. 20, 2022

(54) DEVICES AND METHODS FOR DIAGNOSTICS BASED ON ANALYSIS OF NUCLEIC ACIDS

(71) Applicant: RGA International Corporation, Grand Cayman, KY (US)

(72) Inventors: Benjamin YU, Del Mar, CA (US); Ahmed GHOURI, San Diego, CA (US); Gary RAYNER, Las Vegas, NV (US); Raghu SUGUVANAM, La Jolla, CA (US); Zoltan PAPP, Spicewood, TX (US)

(21) Appl. No.: 17/372,199

(22) Filed: Jul. 9, 2021

Related U.S. Application Data
(63) Continuation of application No. 15/011,036, filed on Jan. 29, 2016, now abandoned.
(60) Provisional application No. 62/110,175, filed on Jan. 30, 2015.

(51) Int. Cl. G16B 30/00; C12Q 1/68; G16H 50/20; G16B 20/20; G16B 50/30; C12Q 1/6874

(52) U.S. Cl. CPC G16B 30/00; C12Q 1/68; G16H 50/20; G16B 50/00; G16B 50/30; C12Q 1/6874; G16B 20/20

(57) ABSTRACT

A condition can be diagnosed based on a symptom experienced by a subject and based on a biological sample including nucleic acids. Based on the symptom, a first set of the nucleic acids can be preselected for analysis. A first plurality of the nucleic acids of the first set that are present in the first biological sample can be captured. For each of the captured nucleic acids of the first plurality, an amount of that captured nucleic acid that is present in the first biological sample can be quantified and sequenced and based on the sequence of that captured nucleic acid, an origin of that captured nucleic acid can be identified. An indication can be output of the quantified amount and the identified origin of at least one captured nucleic acid that is present in the first biological sample.

### CROSS-REFERENCE TO RELATED APPLICATIONS

This application is a continuation application of U.S. application Ser. No. 15/011,036, filed Jan. 29, 2016, which claims the benefit of U.S. Provisional Patent Application No. 62/110,175, filed Jan. 30, 2015 and entitled “Devices and Methods for Diagnostics Based on Analysis of Nucleic Acids,” each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jul. 8, 2021, is named 13617-004-999_SL.txt and is 3,618 bytes in size.

### FIELD

This application relates to devices and methods for diagnostics based on analysis of nucleic acids.

### BACKGROUND

Physicians order diagnostic tests, devices, and procedures to identify the cause of their patients' symptoms. For any particular symptom or like indication of a disease or abnormality, a patient can undergo several different tests, ranging from a simple physical exam to extensive or invasive assays. The use of multiple tests can be time-consuming. Many tests require technical equipment, extensive training, and specialists to perform and interpret each test. As a result, the current state of medical testing is relatively expensive, complicated and inaccessible to millions of patients. Paradoxically, this practice potentially can lead to delayed diagnosis and care. For example, physicians who are pressed to ration tests, may forego ordering a test and miss a diagnosis. New approaches are needed to streamline diagnostic testing and improve accessibility.

An exemplary justification for ordering multiple tests is that some tests evaluate only one process at a time, and as a result, additional tests can be needed to evaluate several possible diagnoses (FIGS. 1A-1B). For example, FIGS. 1A-B illustrate the use of multiple tests for each symptom and an aggregate view of exemplary devices and procedures that can be used for diagnostic testing for an exemplary symptom. FIG. 1A illustrates a current diagnostic paradigm utilizing multiple tests, specialists, and test procedures to evaluate multiple diagnoses. The table illustrated in FIG. 1B lists a relatively common symptom seen by the physician, e.g., chest pain, and its possible causes (or diagnoses). Multiple test choices exist to assist the physician in identifying the possible cause of a patient's symptoms. For example, a given symptom can arise from a particular site (e.g., a particular location of the body), can have an associated diagnosis (e.g., a cause for the symptom), can have an associated pathology (e.g., detectable manifestation). Additionally, one or more tests can be available to assist the physician in making a diagnosis, e.g., by ordering one or more tests that potentially can distinguish among pathologies associated one or more potential diagnoses.

As one example, for the symptom “chest pain” illustrated in FIG. 1B, the symptom potentially can arise from the aorta, which can be associated with a diagnosis of aortic dissection, which can be associated with a pathology of aortic wall damage that can be tested using one or more of CXR, transesophageal echocardiogram, angiography, MRI, or CT). As illustrated in FIG. 1B, the symptom “chest pain” also potentially can arise from the esophagus, which can be associated with a diagnosis of esophagitis, which can be associated with a pathology of esophagus damage that can be tested using one or more of endoscopy, pH, or perfusion test. As illustrated in FIG. 1B, the symptom “chest pain” also potentially can arise from the heart, which can be associated with a diagnosis of angina pectoris, which can be associated with a pathology of heart muscle ischemia that can be tested using one or more of serum troponin, angiography, EKG, or image-perfusion tests; or can be associated with a diagnosis of myocardial infarction, which can be associated with a pathology of heart muscle ischemia that can be tested using one or more of serum troponin, angiography, EKG, or image-perfusion tests; or can be associated with a diagnosis of pericarditis, which can be associated with a pathology of external heart and diaphragm muscle pain that can be tested using one or more of EKG, CXR, CT, or MRI. As illustrated in FIG. 1B, the symptom “chest pain” also potentially can arise from the lung, which can be associated with a diagnosis of pneumonia, which can be associated with a pathology of lung damage and infection that can be tested using one or more of CXR, blood count, or bacterial culture; or can be associated with a diagnosis of pulmonary embolism, which can be associated with a pathology of lung damage and hypoxia that can be tested using one or more of V/Q perfusion scan and angiography. As illustrated in FIG. 1B, the symptom “chest pain” also potentially can arise from the musculoskeletal system, which can be associated with a diagnosis of costochondritis, which can be associated with a pathology of cartilage inflammation that can be suitably tested. As illustrated in FIG. 1B, the symptom “chest pain” also potentially can arise from the stomach, which can be associated with a diagnosis of gastritis, which can be associated with a pathology of gastric tissue damage that can be tested using one or more of endoscopy or biopsy. As illustrated in FIG. 1B, the symptom “chest pain” also rarely arises from the pancreas, which can be associated with a diagnosis of pancreatitis, which can be associated with a pathology of pancreatic tissue damage that can be tested using one or more serum tests, e.g., for lipase or amylase. Accordingly, in this example, approximately twenty tests potentially can be used to evaluate a spectrum of potential causes for chest pain. However, in a practical example, approximately 5-7 tests potentially can be used initially so as to exclude the most life-threatening conditions. If those tests are negative for life-threatening conditions, then the patient can be considered to have another condition that is not yet tested by the initially tests. A lost opportunity can arise if the second round of tests are also negative, and yet a further interrogation of potentially life-threatening conditions may be needed.

There is great interest in using nucleic acids as analytes in medical testing. Nucleic acids, e.g. deoxyribonucleic (DNA) and ribonucleic acids (RNA), are present in every form of life and can be used to distinguish different organisms. DNA and RNA are composed of long polymers of four molecules called nucleotides. These nucleotides differ by nitrogenous bases called cytidine (C), guanine (G), thymine/uracil (T or U), and adenine (A). DNA and RNA vary in nucleotide number and order. For instance, DNA polymers can be relatively short, e.g., can be 5 or fewer nucleotides long, or can be hundreds of millions of nucleotides long, or anywhere in between. The order of nucleotides differs in every organism and can be used to identify human vs. non-human DNA. Specific DNA sequence testing for pathogens is often highly diagnostic and potentially can overcome the difficulty of isolating slow-growing organisms such as fungi and atypical mycobacteria.

Nucleic acid sequences can also be used to identify the anatomical location or cell type of origin. Specific sequences of DNA, called genes, produce RNA, which are used as a template for the cell to produce new proteins and enzymes necessary for the cell's function. For example, FIGS. 2A-2D illustrate an exemplary relationship of symptoms to organ site and cell damage to cell markers. Damaged or altered physiology of organs can be responsible for certain symptoms, e.g., many common symptoms, experienced by patients, including chest pain or abdominal pain. FIG. 2A illustrates some exemplary potential sites responsible for symptoms, which can include the kidney, the blood, the stomach, the lung parenchyma, the lung vascular endothelium, the small and large intestinal epithelium, the cardiac myocyte, or the cardiac atrium or ventricle. Organs can include, or can be composed of, thousands to millions of cells, each of which can have distinct appearances and can produce different internal and/or external products, including proteins, enzymes, and the like. FIG. 2B illustrates some exemplary cell types from different tissues, e.g., red blood cells (RBC), neutrophils, lymphocytes, lung epithelial cells, and cardiomyocytes. Cell-type specific proteins are frequently used in the clinic to identify different cell types, analogously to “name-tags.” A list of protein markers commonly used in the clinic are illustrated in FIG. 2C. Exemplary RBC-specific protein markers include hemoglobin. Exemplary neutrophil-specific protein markers include CD16b and myeloperoxidase. Exemplary T-lymphocyte-specific protein markers include CD3. Exemplary B-lymphocyte-specific protein markers include CD20. Exemplary lung-specific protein markers include surfactant proteins. Exemplary heart-specific protein markers include atrial natriuretic peptide and troponin T protein. Biological samples such as blood can include multiple types of intact cells. For example, FIG. 2D illustrates exemplary detection of cells, bacteria, viruses, or necrotic cells in biological fluids. For example, the left panel of FIG. 2D illustrates exemplary intact, live cells (cellular response). If cell damage is present, internal components of cells such as proteins, DNA, and RNA potentially can be found circulating externally from the cell. For example, the middle panel of FIG. 2D illustrates exemplary evidence of tissue damage, e.g., extracellular cardiac proteins, cardiac DNA, or cardiac RNA from a damaged cardiomyocyte. There may also be other or “foreign” organisms in biological samples, such as bacteria, viruses, or fungi, such as illustrated in the right panel of FIG. 2D. In this case, foreign DNA and RNA molecules can be present.

Additionally, because the cells in the human body perform their respective, different functions, the cells' proteins and corresponding RNAs can be used to identify different cell types. For example, RNAs made in the heart can be used to distinguish heart tissue from lung tissue. The production of RNA is a highly regulated process. During this process, specific areas of the genome, e.g., genes, can gather large molecules to produce RNA (e.g. RNA polymerase, transcription factors, elongation, splicing factors), control RNA splicing, modify DNA or RNA directly, and alter DNA accessibility, the latter of which can modify DNA packaging proteins called histones. The production of RNA from or the association of transcriptional machinery with DNA from these sites can be used as evidence for an active gene. Changes in gene activity can be associated with different cell types and cell responses to a number of conditions such as disease, cell damage, ischemia, nutritional changes, chemical or drug exposure, and the like. Thus, active genes, specific cell types, and different organisms potentially can be ascertained through the detection of specific DNA and RNA sequences and specific chemical modifications such as methylation. See, for example, Rando et al., “Genome-wide views of chromatin structure,” Annu. Rev. Biochem. 78: 245-271 (2009), the entire contents of which are incorporated by reference herein.

For example, FIGS. 3A-3B illustrate exemplary cell-type specific products, proteins are produced by active genes. In FIG. 3A, cell-type specific products are produced by active genes. The promoter acts like a switch to turn “on” a gene. An active gene then produces RNA which is used to manufacture the final product (dashed lines). The promoter itself is under the control of signals (solid lines) from enhancers (A1) and other regulators (A2). These signals can modify histone proteins that underlie these regions of DNA. These histone changes, which can include, for example, acetylation (ac) and tri-methylation (me3), can be captured by antibodies and their associated DNA analyzed in a procedure called chromatin immunoprecipitation. For further details, see Ren et al., “Use of chromatin immunoprecipitation assays in genome-wide location analysis of mammalian transcription factors,” Methods Enzymol. 376: 304-315 (2004), the entire contents of which are incorporated by reference herein. For example, in the nonlimiting example illustrated in FIG. 3A, the enhancer has the nucleic acid sequence ATATGAGGCTAGGGAA (SEQ ID NO: 1) and histone changes in the active gene cause lysine 27 to be acetylated (H3K27ac); the promoter has the nucleic acid sequence TATACTCCGATCCCTT (SEQ ID NO: 2) and hi stone changes in the active gene cause lysine 4 to be methylated (H3K4me3); and the gene has the sequence GTGGTATGATGGGTGC (SEQ ID NO: 3) and histone changes in the active gene cause lysine 36 to be methylated (H3K36me3). The table illustrated in FIG. 3B summarizes exemplary types of assays that can detect “active” genes, such as capturing modified histones, e.g., in the present, nonlimiting example, capture of H3K27ac, capture of H3K4me3, capture of H3K36me3, as well as capture of RNA polymerase or RNA sequencing (RNA-seq). In addition to detecting which RNAs are produced, the capture of modified histones, proteins involved in gene regulation, accessibility of DNA or RNA, DNA or RNA modifications, and the presence of enhancer, promoter, or gene DNA sequences can be used to identify active genes. FIG. 3B also summarizes exemplary types of assays that can be used to identify inactive genes.

Methods for nucleic acid analysis such as high-throughput sequencing have improved immensely and are capable of detecting millions of DNA or RNA molecules in one assay and potentially identifying some or all known pathogens and genes. Success of these approaches are often measured by the torrent of data that has been obtained from nucleic acid sequencing and by the estimates that petabytes of new data will be generated annually through this methodology. Despite this enormous potential, nucleic acid-based testing remains highly specialized and restricted to narrow uses. Some obstacles include technical complexity or technical difficulties. For example, high-throughput sequencing can require a series of highly specialized personnel with non-interchangeable skills who are responsible for sample collection, nucleic acid extraction and preparation, sequencing, data transfer, sequence data conversion, and reporting. In addition, the amount of data produced from sequencing billions of molecules can be memory- and processor-intensive, making data transfer and analyses extremely challenging. Furthermore, the interpretation of sequence data can be based on artificial intelligence, supercomputer-based machine learning, and consortium-based discovery to organize and understand the sequence output. These complexities can create barriers for use of nucleic acid sequencing in the clinic, where the expertise and time necessary to generate or interpret vast amounts of data are unavailable to most providers and hospitals.

### SUMMARY

Embodiments of the present invention provide devices and methods for diagnostics based on analysis of nucleic acids.

Under one aspect, a method is provided for use in diagnosing a condition based on a symptom experienced by a subject and based on a first biological sample obtained from the subject, the first biological sample including nucleic acids, the method being executed by a device. The method can include, based on the symptom, preselecting a first set of the nucleic acids for analysis. The method also can include capturing by the device a first plurality of the nucleic acids of the first set that are present in the first biological sample. The method also can include, for each of the captured nucleic acids of the first plurality: quantifying by the device an amount of that captured nucleic acid that is present in the first biological sample; sequencing by the device that captured nucleic acid; and based on the sequence of that captured nucleic acid, identifying by the device an origin of that captured nucleic acid. The method also can include outputting by the device an indication of the quantified amount and the identified origin of at least one captured nucleic acid that is present in the first biological sample.

Optionally, preselecting the first set of the nucleic acids for analysis includes receiving by the device a first symptom-specific cartridge including a first set of complementary nucleic acids configured to capture the first set of the nucleic acids for analysis. Optionally, the method further includes, after the outputting step, removing the first symptom-specific cartridge from the device and receiving by the device a second symptom-specific cartridge including a second set of complementary nucleic acids. Optionally, the first set of complementary nucleic acids is different than the second set of complementary nucleic acids.

Additionally, or alternatively, the method optionally can include outputting by the device an indication of the quantified amount of each of the captured nucleic acids of the first plurality.

Additionally, or alternatively, the capturing can include separating extracellular nucleic acids in the first biological sample from intracellular nucleic acids in the first biological sample; and the quantifying and sequencing steps can be performed separately on the separated extracellular nucleic acids and on the intracellular nucleic acids. Optionally, the method includes outputting by the device an indication of the quantified amount of at least one of the extracellular nucleic acids and an indication of the quantified amount of at least one of the intracellular nucleic acids.

Additionally, or alternatively, the identifying by the device the origin of the captured nucleic acid can include comparing the sequence of that nucleic acid to sequences stored in a library stored in a computer-readable medium of the device. Optionally, the library stores nucleic acid sequences for a human and for a plurality of pathogens. Optionally, the output indicates the relative number of a pathogen per human cell.

Additionally, or alternatively, the method optionally includes receiving by the device a second biological sample obtained from the subject, the second biological sample being different from the first biological sample; and capturing by the device a second plurality of the nucleic acids of the first set that are present in the second biological sample. Optionally, for each of the captured nucleic acids of the second plurality, the method also can include quantifying by the device an amount of that captured nucleic acid that is present in the second biological sample; sequencing by the device that captured nucleic acid; and based on the sequence of that captured nucleic acid, identifying by the device an origin of that captured nucleic acid. Optionally, the outputting by the device further includes an indication of the quantified amount and the identified origin of at least one captured nucleic acid that is present in the second biological sample.

Additionally, or alternatively, the method optionally further can include outputting by the device an indication of at least one potential diagnosis for the subject and an indication of the likelihood of the at least one potential diagnosis based on the quantified amount and the identified origin of at least one captured nucleic acid that is present in the first biological sample.

Under another aspect, a device is provided for use in diagnosing a condition based on a symptom experienced by a subject and based on a first biological sample obtained from the subject, the first biological sample including nucleic acids. The device can include a first set of complementary nucleic acids configured to capture a first set of the nucleic acids, the first set of the nucleic acids being selected based on the symptom, the first set of complementary nucleic acids capturing a first plurality of the nucleic acids of the first set that are present in the first biological sample. The device also can include a nucleic acid quantifier configured to quantify an amount of each of the captured nucleic acids that is present in the first biological sample. The device also can include a nucleic acid sequencer configured to sequence each captured nucleic acid that is present in the first biological sample. The device also can include a processor coupled to the quantifier and to the sequencer and being suitably programmed to identify an origin of each captured nucleic acid based on the sequence of that captured nucleic acid. The device also can include an output module coupled to the processor, the processor further being suitably programmed to cause the output module to output an indication of the quantified amount and the identified origin of at least one captured nucleic acid that is present in the first biological sample.

Optionally, the device includes a receptacle configured to receive the first set of complementary nucleic acids within a first symptom-specific cartridge. Optionally, the first symptom-specific cartridge is removable from the receptacle and replaceable with a second symptom-specific cartridge including a second set of complementary nucleic acids. Optionally, the first set of complementary nucleic acids is different than the second set of complementary nucleic acids.

Additionally, or alternatively, the processor further can be suitably programmed to cause the output module to output an indication of the quantified amount of each of the captured nucleic acids of the first plurality.

Additionally, or alternatively, the device further can include a separator configured to separate extracellular nucleic acids in the first biological sample from intracellular nucleic acids in the first biological sample. Optionally, the nucleic acid quantifier and nucleic acid sequencer separately operate on the separated extracellular nucleic acids and on the intracellular nucleic acids. Optionally, the processor further is suitably programmed to cause the output module to output an indication of the quantified amount of at least one of the extracellular nucleic acids and an indication of the quantified amount of at least one of the intracellular nucleic acids.

Additionally, or alternatively, the device optionally further can include a computer-readable medium coupled to the processor. The processor optionally further can be suitably programmed to identify the origin of the captured nucleic acid based on comparing the sequence of that nucleic acid to sequences stored in a library stored in the computer-readable medium. Optionally, the library stores nucleic acid sequences for a human and for a plurality of pathogens. Optionally, the output indicates the relative number of a pathogen per human cell.

Additionally, or alternatively, the first set of complementary nucleic acids optionally can be configured to capture a second plurality of the nucleic acids of the first set that are present in a second biological sample obtained from the subject, the second biological sample being different from the first biological sample. Optionally, the nucleic acid quantifier further can be configured to quantify an amount of each of the captured nucleic acids that is present in the second biological sample. Optionally, the nucleic acid sequencer further can be configured to sequence each of the captured nucleic acids that is present in the second biological sample. Optionally, the processor further can be suitably programmed to identify an origin of each captured nucleic acid based on the sequence of the captured nucleic acid that is present in the second biological sample. Optionally, the processor further can be suitably programmed to cause the output module to output an indication of quantified amount and the identified origin of at least one captured nucleic acid that is present in the second biological sample.

Additionally, or alternatively, the processor optionally further can be suitably programmed to cause the output module to output an indication of at least one potential diagnosis for the subject and an indication of the likelihood of the at least one diagnosis based on the quantified amount and the identified origin of at least one captured nucleic acid that is present in the first biological sample.

Under yet another aspect, a database can be stored in a computer-readable medium. The database can store at least a plurality of symptoms, a nucleic acid sequence associated with each of the symptoms, a potential diagnosis associated with each of the symptoms, a laboratory test or a procedure for each of the symptoms, and an inferred value for each of the symptoms, the inferred value including a clinical inference based on a result of said laboratory test for the respective symptom.

Under another aspect, a method is provided of generating a database stored in a computer-readable medium. The method can include receiving, by a device, a plurality of medical documents, each document describing at least one symptom experienced by a respective patient, a laboratory test or a procedure performed on that patient, and a diagnosis associated with the at least one symptom experienced by that patient, the diagnosis being based on a result of the laboratory test performed on that patient. The method also can include, by the device, inferring values based on the symptoms, the laboratory tests, and the diagnoses described in the plurality of medical documents, each inferred value including a clinical inference based on a result of at least one of the laboratory tests for the respective symptom. The method also can include, by the device, identifying a nucleic acid test value associated with each of the inferred values. The method also can include, by the device, generating and storing in the computer-readable medium a plurality of database entries, each database entry of the plurality including a symptom, a laboratory test or a procedure performed on a patient having that symptom, at least one possible diagnosis associated with that symptom, an inferred value for that diagnosis, and a nucleic acid test value for that inferred value.

Optionally, the nucleic acid test value includes an RNA sequence or a DNA sequence. Additionally, or alternatively, the nucleic acid test values optionally include one or more specific nucleic acid sequences, one or more groups of nucleic acid sequences, one or more quantities of nucleic acid sequences, one or more patterns of nucleic acid sequences, or one or more contexts of nucleic acid sequences. Optionally, the one or more contexts of nucleic acid sequences include one or more associations of nucleic acid sequences with chemical modifications, proteins, other intramolecular or extramolecular nucleic acids, or intracellular or extracellular subcompartments.

Additionally, or alternatively, the plurality of medical documents optionally include standard medical codes describing at least some of the symptoms, laboratory tests or procedures, and diagnoses. Additionally, or alternatively, the plurality of medical documents further include physical findings, medications, or environmental exposures.

Under still another aspect, a method is provided for performing one or more nucleic acid tests based on one or more symptoms experienced by a patient. The method can include receiving by a device respective identifiers of the one or more symptoms experienced by the patient. The method also can include, by the device, submitting to a database a query based on the respective identifiers of each of the one or more symptoms. The database can include a computer-readable medium storing at least a plurality of symptoms, a nucleic acid sequence associated with each of the symptoms, a potential diagnosis associated with each of the symptoms, a laboratory test or a procedure for each of the symptoms, and inferred data for each of the symptoms, the inferred value including a clinical inference based on a result of said laboratory test for the respective symptom. The method also can include, by the device, receiving from the database a response to the query, the response including one or more nucleic acid tests based on the nucleic acid sequences respectively associated with the one or more symptoms identified in the query. The method also can include, by the device, outputting respective representations of the one or more nucleic acid tests. The method also can include receiving, by a receptacle of the device, a cartridge configured to perform at least one of the one or more nucleic acid tests.

Optionally, the method further includes, by the device, outputting a result of the at least one of the one or more nucleic acid tests. The result can include a count of RNA or DNA of the subject or of a pathogen in the subject, the RNA or DNA having the nucleic acid sequence associated with at least one of the one or more symptoms identified in the query.

Additionally, or alternatively, the response to the query can include a representation of a plurality of nucleic acid tests based on a plurality of nucleic acid sequences respectively associated with the one or more symptoms identified in the query. The cartridge optionally can be configured to perform each nucleic acid test of the plurality.

The method optionally can include receiving, by a receptacle of the device, at least one additional cartridge, the at least one additional cartridge being configured to perform at least one other of the nucleic acid tests.

Additionally, or alternatively, the method optionally can include performing by the device the at least one of the one or more nucleic acid tests. Optionally, the performing can include: quantifying by the device an amount of a first subset of the nucleic acids that are present in the biological sample, the first subset of the nucleic acids having a first origin; quantifying by the device an amount of a second subset of the nucleic acids that are present in the biological sample, the second subset of the nucleic acids having a second origin; and determining by the device at least one possible diagnosis based on the amount of the first subset of the nucleic acids and based on the amount of the second subset of the nucleic acids. The method optionally can include outputting by the device an indication of the at least one possible diagnosis. The method optionally, can include, by the device, receiving an indication of at least one of: a diagnosis made by the caregiver, a result of a laboratory test or a procedure performed on the subject, a symptomatic code, a site of injury, a cellular response, a host-immune response, a contribution of a non-human organism, or an origin of cells or symptoms. The method optionally can include transmitting by the device to the database the received indication for use in updating the database.

Optionally, the method further can include receiving by the device or by a second device respective identifiers of one or more symptoms experienced by a second patient. The symptoms experienced by the second patient can be the same as the symptoms experienced by the first patient. Optionally, the method further can include, by the device or by the second device, submitting to the updated database a second query based on the respective identifiers of each of the one or more symptoms. Optionally, the method further can include, by the device or by the second device, receiving from the updated database a response to the second query, the response including one or more updated nucleic acid tests based on the nucleic acid sequences respectively associated with the one or more symptoms identified in the second query. At least one of the one or more updated nucleic acid tests can be different than at least one of the one or more nucleic acid tests. Optionally, the method further can include, by the device or by the second device, outputting respective representations of the updated one or more nucleic acid tests. Optionally, the method further can include receiving, by the receptacle of the device or by a receptacle of the second device, a second cartridge configured to perform at least one of the updated one or more nucleic acid tests.

Under yet another aspect, a device is provided for performing one or more nucleic acid tests based on one or more symptoms experienced by a patient. The device can include an input module configured to receive respective identifiers of the one or more symptoms experienced by the patient. The device also can include a query module configured to submit to a database a query including the respective identifiers of each of the one or more symptoms. The database can include a computer-readable medium storing at least a plurality of symptoms, a nucleic acid sequence associated with each of the symptoms, a potential diagnosis associated with each of the symptoms, a laboratory test or a procedure for each of the symptoms, and inferred data for each of the symptoms, the inferred value including a clinical inference based on a result of said laboratory test for the respective symptom. The query module further can be configured to receive from the database a response to the query, the response including one or more nucleic acid tests based on the nucleic acid sequences respectively associated with the one or more symptoms identified in the query. The device further can include an output module configured to output respective representations of the one or more nucleic acid tests. The device further can include a receptacle configured to receive a cartridge configured to perform at least one of the one or more nucleic acid tests.

Optionally, the output module further can be configured to output a result of the at least one of the one or more nucleic acid tests, the result including a count of RNA or DNA of the subject or of a pathogen in the subject, the RNA or DNA having the nucleic acid sequence associated with at least one of the one or more symptoms identified in the query.

Additionally, or alternatively, the response to the query optionally can include a representation of plurality of nucleic acid tests based on a plurality of nucleic acid sequences respectively associated with the one or more symptoms identified in the query, the cartridge being configured to perform each nucleic acid test of the plurality.

Optionally, the receptacle of the device can be configured to receive least one additional cartridge, the at least one additional cartridge being configured to perform at least one other of the nucleic acid tests.

Additionally, or alternatively, the cartridge optionally can include a first nucleic acid capture module configured to capture a first subset of the nucleic acids that are present in the biological sample, the first subset of the nucleic acids having a first origin. The cartridge optionally further can include a second nucleic acid capture module configured to capture a second subset of the nucleic acids that are present in the biological sample, the second subset of the nucleic acids having a second origin. Optionally, the device further can include a nucleic acid quantifier configured to quantify a respective amount of each of the first and second subsets of captured nucleic acids. The device optionally further can include a diagnosis module configured to determine at least one possible diagnosis based on the amount of the first subset of the nucleic acids and based on the amount of the second subset of the nucleic acids. Optionally, the output module can be configured to output an indication of the at least one possible diagnosis. Optionally, the input module further can be configured to receive an indication of at least one of: a diagnosis, a result of a laboratory test or a procedure performed on the subject, a symptomatic code, a site of injury, a cellular response, a host-immune response, a contribution of a non-human organism, or an origin of cells or symptoms. Optionally, the query module further can be configured to transmit by the device to the database the received indication for use in updating the database.

Optionally, the input module further can be configured to receive respective identifiers of one or more symptoms experienced by a second patient. The symptoms experienced by the second patient can be the same as the symptoms experienced by the first patient. The query module optionally further can be configured to submit to the updated database a second query based on the respective identifiers of each of the one or more symptoms. The query module optionally further can be configured to receive from the updated database a response to the second query, the response including one or more updated nucleic acid tests based on the nucleic acid sequences respectively associated with the one or more symptoms identified in the second query. At least one of the one or more updated nucleic acid tests can be different than at least one of the one or more nucleic acid tests. The output module optionally further can be configured to output respective representations of the updated one or more nucleic acid tests. Optionally, the receptacle of the device further can be configured to receive a second cartridge configured to perform at least one of the updated one or more nucleic acid tests.

Under still another aspect, a method is provided for use in diagnosing a condition based on a symptom experienced by a subject and based on a biological sample obtained from the subject, the biological sample including nucleic acids, the method being executed by a device. The method can include, over a first period of time, quantifying by the device an amount of a first subset of the nucleic acids that are present in the biological sample, the first subset of the nucleic acids having a first origin. The method also can include, over the first period of time, quantifying by the device an amount of a second subset of the nucleic acids that are present in the biological sample, the second subset of the nucleic acids having a second origin that is different than the first origin. The method also can include outputting by the device an indication of the amount of the first subset of the nucleic acids quantified over the first period of time. The method also can include outputting by the device an indication of the amount of the second subset of the nucleic acids quantified over the first period of time.

Optionally, the method further includes, based on the amount of the first subset of the nucleic acids quantified over the first period of time, estimating by the device a first likelihood that the subject is suffering from a first condition. The method optionally further can include, based on the amount of the second subset of the nucleic acids quantified over the second period of time, estimating by the device a second likelihood that the subject is suffering from a second condition that is different than the first condition. The method optionally further can include outputting by the device an indication of the first likelihood and an indication of the second likelihood. Optionally, the method further includes, based on the amount of the first subset of the nucleic acids quantified over the first period of time, estimating by the device a first trajectory of an amount of the first subset of the nucleic acids over a second period of time. Optionally, the method further includes, based on the amount of the second subset of the nucleic acids quantified over the first period of time, estimating by the device a second trajectory of an amount of the second subset of the nucleic acids over the second period of time. Optionally, the method further includes outputting by the device an indication of the first trajectory and an indication of the second trajectory. Optionally, the method further includes, based on the first and second trajectories, estimating by the device a second time at which the first or second condition is sufficiently likely as to make a diagnosis that the patient is suffering from that condition; and outputting by the device an indication of the second time.

Additionally, or alternatively, the method further can include receiving by the device additional clinical information regarding the patient. The first and second likelihoods optionally can be further based on the received additional clinical information.

Additionally, or alternatively, the method optionally further can include, over a second period of time subsequent to the first period of time, quantifying by the device an amount of the first subset of the nucleic acids that are present in the biological sample. The method optionally further can include, over the second period of time, quantifying by the device an amount of the second subset of the nucleic acids that are present in the biological sample. The method optionally further can include outputting by the device an indication of the amount of the first subset of the nucleic acids quantified over the second period of time. The method optionally further can include outputting by the device an indication of the amount of the second subset of the nucleic acids quantified over the second period of time.

Additionally, or alternatively, the indications of the amounts of the first and second subsets of nucleic acids quantified over the first period of time optionally can include a histogram.

Additionally, or alternatively, the indication of the amount of the first subset of the nucleic acids over the first period of time optionally can include a number of first cell equivalents. The indication of the amount of the second subset of the nucleic acids over the first time can include a number of second cell equivalents. Optionally, the first origin can include a pathogen, and the number of first cell equivalents can represent a severity of infection of the subject by the pathogen. Additionally, or alternatively, the number of first cell equivalents or the number of second cell equivalents optionally can represent a severity of a condition from which the subject is suffering or clinical significance. Additionally, or alternatively, the number of first cell equivalents or the number of second cell equivalents optionally can represent a response to a treatment.

Additionally, or alternatively, the method optionally can include, based on the amount of the first subset of the nucleic acids quantified over the first period of time, ceasing quantifying by the device an amount of the first subset of the nucleic acids over a second period of time that is subsequent to the first period of time. The method further optionally can include, based on the ceasing, over the second period of time, quantifying by the device an amount of a third subset of the nucleic acids that are present in the biological sample, the third subset of the nucleic acids having a third origin that is different than the first origin and that is different than the second origin. The method further optionally can include outputting by the device an indication of the amount of the third subset of the nucleic acids quantified over the second period of time.

Optionally, the device includes a sequencer that quantifies the first subset of the nucleic acids over the first period of time and that is reassigned so as to quantify the third subset of the nucleic acids over the second period of time. Additionally, or alternatively, the ceasing optionally can be based on an estimation by the device of a first likelihood that the subject is suffering from a first condition, the estimation being based on the amount of the first subset of the nucleic acids quantified over the first period of time. Optionally, the ceasing further can be based on a comparison by the device of the estimation to a threshold.

Under yet another aspect, a device is provided for use in diagnosing a condition based on a symptom experienced by a subject and based on a biological sample obtained from the subject, the biological sample including nucleic acids. The device can include a first quantification module configured to quantify, over a first period of time, an amount of a first subset of the nucleic acids that are present in the biological sample, the first subset of the nucleic acids having a first origin. The device also can include a second quantification module configured to quantify, over the first period of time, an amount of a second subset of the nucleic acids that are present in the biological sample, the second subset of the nucleic acids having a second origin that is different than the first origin. The device also can include an output module configured to: output an indication of the amount of the first subset of the nucleic acids quantified over the first period of time, and to output an indication of the amount of the second subset of the nucleic acids quantified over the first period of time.

Optionally, the device further can include an estimation module configured to estimate, based on the amount of the first subset of the nucleic acids quantified over the first period of time, a first likelihood that the subject is suffering from a first condition. Optionally, the estimation module further can be configured to estimate, based on the amount of the second subset of the nucleic acids quantified over the second period of time, a second likelihood that the subject is suffering from a second condition that is different than the first condition. Optionally, the output module further can be configured to output an indication of the first likelihood and an indication of the second likelihood. Optionally, the estimation module further can be configured to estimate, based on the amount of the first subset of the nucleic acids quantified over the first period of time, a first trajectory of an amount of the first subset of the nucleic acids over a second period of time. Optionally, the estimation module further can be configured to estimate, based on the amount of the second subset of the nucleic acids quantified over the first period of time, a second trajectory of an amount of the second subset of the nucleic acids over the second period of time. Optionally, the output module further can be configured to output an indication of the first trajectory and an indication of the second trajectory. Optionally, the estimation module further is configured to estimate, based on the first and second trajectories, a second time at which the first or second condition is sufficiently likely as to make a diagnosis that the patient is suffering from that condition. Optionally, the output module further is configured to output an indication of the second time.

Additionally, or alternatively, the device optionally further can include an input interface configured to receive additional clinical information regarding the patient. The first and second likelihoods optionally further can be based on the received additional clinical information.

Additionally, or alternatively, the first quantification module optionally can be configured to quantify, over a second period of time subsequent to the first period of time, an amount of the first subset of the nucleic acids that are present in the biological sample. The second quantification module optionally can be configured to quantify, over the second period of time, an amount of a second subset of the nucleic acids that are present in the biological sample. Optionally, the output module can be configured to output an indication of the amount of the first subset of the nucleic acids quantified over the second period of time. Optionally, the output module can be configured to output an indication of the amount of the second subset of the nucleic acids quantified over the second period of time.

Additionally, or alternatively, the indications of the amounts of the first and second subsets of nucleic acids quantified over the first period of time optionally can include a histogram.

Additionally, or alternatively, the indication of the amount of the first subset of the nucleic acids over the first period of time optionally can include a number of first cell equivalents, and the indication of the amount of the second subset of the nucleic acids over the first time optionally can include a number of second cell equivalents. Optionally, the first origin includes a pathogen, and the number of first cell equivalents represents a severity of infection of the subject by the pathogen. Additionally, or alternatively, the number of first cell equivalents or the number of second cell equivalents optionally represents a severity of a condition from which the subject is suffering or clinical significance. Additionally, or alternatively, the number of first cell equivalents or the number of second cell equivalents optionally represents a response to a treatment.

Additionally, or alternatively, the first quantification module optionally can be configured to cease, based on the amount of the first subset of the nucleic acids quantified over the first period of time, quantifying an amount of the first subset of the nucleic acids over a second period of time that is subsequent to the first period of time. Optionally, the first quantification module can be configured to quantify, based on the ceasing, over the second period of time, an amount of a third subset of the nucleic acids that are present in the biological sample, the third subset of the nucleic acids having a third origin that is different than the first origin and that is different than the second origin. Optionally, the output module further can be configured to output an indication of the amount of the third subset of the nucleic acids quantified over the second period of time.

Additionally, or alternatively, the first quantification module optionally includes a sequencer that quantifies the first subset of the nucleic acids over the first period of time and that is reassigned so as to quantify the third subset of the nucleic acids over the second period of time. Additionally, or alternatively, the ceasing optionally can be based on an estimation by the device of a first likelihood that the subject is suffering from a first condition, the estimation being based on the amount of the first subset of the nucleic acids quantified over the first period of time. Optionally, the ceasing further can be based on a comparison by the device of the estimation to a threshold.

Under yet another aspect, a method is provided for use in assessing the quality of a biological sample obtained from a subject, the biological sample including nucleic acids, the method being executed by a device. The method can include quantifying by the device an amount of a first subset of the nucleic acids that are present in the biological sample, the first subset of the nucleic acids having an intracellular origin. The method further can include quantifying by the device an amount of a second subset of the nucleic acids that are present in the biological sample, the second subset of the nucleic acids having an extracellular origin. The method further can include outputting by the device an indication of the amount of the first subset of the nucleic acids. The method further can include outputting by the device an indication of the amount of the second subset of the nucleic acids. The relative amounts of the first and second subsets of the nucleic acids can indicate the quality of the biological sample.

Optionally, the method further can include outputting by the device an indication of an expected amount of the first subset of the nucleic acids in a normal biological sample and an indication of an expected amount of the second subset of the nucleic acids in a normal biological sample.

Under still another aspect, a device is provided for use in assessing the quality of a biological sample obtained from a subject, the biological sample including nucleic acids. The device can include a first quantification module configured to quantify an amount of a first subset of the nucleic acids that are present in the biological sample, the first subset of the nucleic acids having an intracellular origin. The device further can include a second quantification module configured to quantify an amount of a second subset of the nucleic acids that are present in the biological sample, the second subset of the nucleic acids having an extracellular origin. The device further can include an output module configured to output an indication of the amount of the first subset of the nucleic acids and to output an indication of the amount of the second subset of the nucleic acids. The relative amounts of the first and second subsets of the nucleic acids can indicate the quality of the biological sample.

Optionally, the output module further is configured to output an indication of an expected amount of the first subset of the nucleic acids in a normal biological sample and an indication of an expected amount of the second subset of the nucleic acids in a normal biological sample.

### BRIEF DESCRIPTION OF DRAWINGS

FIGS. 1A-1C illustrate an exemplary overview of current diagnostic paradigm and goals of a universal diagnostic test. FIG. 1A illustrates a current diagnostic paradigm utilizing multiple tests, specialists, and test procedures to evaluate multiple diagnoses. FIG. 1B illustrates an array of tests and procedures used to evaluate an exemplary symptom, chest pain; multiple test choices exist to assist the physician in identifying the possible diagnoses responsible for a patient's symptoms. FIG. 1C illustrates use of nucleic acid-based diagnostics to replace multiple tests, according to some embodiments of the present invention.

FIGS. 2A-2D illustrate an exemplary relationship of symptoms to organ site and cell damage to cell markers. FIG. 2A illustrates some potential sites responsible for symptoms; damaged or altered physiology of organs can be responsible for many common symptoms experienced by patients, including chest pain or abdominal pain. FIG. 2B schematically illustrates cell types from different tissues; organs include or are composed of thousands to millions of cells, each of which have distinct appearances and produce different internal and external products, including proteins, enzymes, etc. FIG. 2C illustrates exemplary cell type-specific products/proteins; cell-type specific proteins are frequently used in the clinic to identify different cell types, analogous to “name-tags;” a list of commonly used protein markers in the clinic are shown. FIG. 2D illustrates detection of cells, bacteria, viruses, and necrotic cells in biological fluids; biological samples like blood can contain multiple types of intact cells (left panel), and if cell damage is present, internal components of cells such as proteins, DNA and RNA, might be found circulating externally from the cell (middle panel); there may also be other organisms in biological samples, such as bacteria, viruses and fungi (right panel); in this case, foreign DNA and RNA molecules will be present.

FIGS. 3A-3B illustrate exemplary cell-type specific products, proteins produced by active genes. FIG. 3A illustrates exemplary cell-type specific products being produced by active genes. The promoter acts like a switch to turn “on” a gene. An active gene then produces RNA which is used to manufacture the final product (dashed lines). The promoter itself is under the control of signals (solid lines) from enhancers (1) and other regulators (2). These signals modify histone proteins (gray) which underlie these regions of DNA. These histone changes including acetylation (ac) and tri-methylation (me3) can be captured by antibodies and their associated DNA analyzed in a procedure called chromatin immunoprecipitation. The final result of an active gene is that RNA is produced (3). DNA sequences disclosed as SEQ ID NOS 1-3, respectively, in order of appearance, and RNA sequence disclosed as SEQ ID NO: 8. FIG. 3B illustrates a table that summarizes exemplary types of assays which can detect “active” genes. In addition to detecting which RNAs are produced, the capture of modified histones and the presence of enhancer, promoter, or gene DNA sequences can be used to identify active genes. Shown are also methods to identify inactive genes.

FIGS. 4A-4C illustrate exemplary relationships between different types of clinical data, according to some embodiments. FIG. 4A illustrates a diagnostic algorithm used by physicians utilizing physical exams, procedures and laboratory tests to draw inferences about the cause of a patient's symptoms. FIG. 4B illustrates the co-occurrence of diagnoses, procedures, and tests in the same document such as medical claim link related data. Because these tests and procedures generate inferred values, this index can be amended to include inferred values. FIG. 4C illustrates examples of inferred diagnostic data, organized by categories and associated values. This index allows for tests and procedures with similar clinical inferences to be recognized and grouped (see FIGS. 5A-5B).

FIGS. 5A-5B illustrate exemplary generation of linked clinically informative sequences through common inferred data, according to some embodiments. Inferred values link clinically informative sequences to tests, procedures, and diagnoses. FIG. 5A illustrates an example of how inferred data (marked with an asterisk) can be used to link procedures or lab tests to specific nucleic acid test sequences (SEQ ID NOS 4, 9, 5 and 7, respectively, in order of appearance), according to some embodiments. FIG. 5B illustrates a current diagnostic algorithm used by physicians utilize physical exams, procedures and laboratory tests to draw inferences about the cause of a patient's symptoms. Inferred data (dashed area) can be categorized as anatomical location of disease (site), cellular response (response), micro-organism or pathogen (micro) detection. According to some embodiments, the same inferred data can be derived from either nucleic acid values.

FIGS. 6A-6C illustrate an overview of an exemplary automated portable or stationary nucleic acid diagnostic device to test multiple diagnoses, according to some embodiments. FIG. 6A illustrates exemplary components allowing for automated sample preparation, sequencing and clinical interpretation, according to some embodiments. These components are configured to perform the following functions: component 1 receives different types of biological fluids, component 2 prepares and enriches biological specimens, component 3 performs sequence-specific capture, component 4 performs sequencing, component 5 performs sequence identification and quantification, component 6 is a display and input components which interacts with the user, component 7 provides connectivity to electronic medical records, external sequence analysis, or data transfer, component 8 involves power source for portable use, component 9 includes portions of device which can be exchanged allowing for optimization, customization and restoration. Inset demonstrates how display and input can be used on the top of the device and location of exchangeable reagents. FIG. 6B illustrates an exemplary stationary configuration detailing possible layout of components into modules and exchangeable compartments, according to some embodiments. FIG. 6C illustrates a stand-alone nucleic acid diagnostic device configured to function to allow the clinician to perform real-time detection of nucleic acid sequences and perform diagnostic interpretation of data integrated with the electronic record and intuition of the provider, according to some embodiments.

FIG. 7 illustrates a detailed overview of exemplary internal components involved in sample preparation and analysis, according to some embodiments. In some embodiments, component 1 allows for different types of sample inputs, e.g. blood, urine, etc., and receives feedback from DNA, RNA sensors. In some embodiments, component 2 separates intact cells, extracellular particles and liquids; lyses cells; extracts; cleans and fragments DNA and RNA. In some embodiments, components 3 and 4 quantify DNA and RNA (oval); electronically report to component 1; capture and/or perform targeted sequencing. In some embodiments, components 5A and 5B respectively perform DNA and RNA analysis by comparing test data to a pre-computed index of sequences representing species, cell types, and host responses pertinent to the patient's symptoms. In addition, in some embodiments, components 5A and 5B can serve to quantify and normalize results. In some embodiments, component 6 is or includes an electronic interface for real-time monitoring of results, for network- and geographical location updating, for interactions with physician and for assistance in diagnostic interpretation. In some embodiments, component 9 is or includes an exchangeable portion of the device which is used for off-site analysis. Off-site, the portions are used to gather and aggregate patient outcome data from the device and from the medical record; to perform optimizations through analysis of concordant and discordant outcomes; and to guide modifications to components 2, 3, 5, and 6 for improved accuracy and sensitivity.

FIG. 8 illustrates an exemplary physical layout of components used to receive and sequence biological samples, according to some embodiments. FIG. 8 describes adjoining components involving existing methodologies to process samples and nucleic acids, according to some embodiments. In some embodiments, in component 2, biological specimens are stored in sample reservoirs and enter into microfluidic chambers by passive, negative or positive pressures, or other means. Within varying caliber channels, cells and particles can be separated or affinity captured. DNA and RNA are released as cells encounter chambers with lytic agents and localized heat or vibration. In some embodiments, in component 3, free DNA and RNA molecules are selectively captured (bead-captured RNA/DNA) and moved to an area involved in sequencing prep reactions, e.g. ligation of adaptors. In some embodiments, these select nucleic acids are sequenced by component 4. In some embodiments, component 5 involves or includes an internal computer, which receives electronic data from component 4 and determines the identity of and counts detected nucleic acids using an internal sequence lookup database.

FIGS. 9A-9B illustrate exemplary configurations of programmable assignment of biological samples to one or more sequencers, according to some embodiments. In FIGS. 9A-9B, component 5 recognizes nucleic acid results from multiple sources and assigns sequencers to samples. In FIG. 9A, component 5 acts as a computer to transfer data from sequencer components (component 4) and accounts for the source of the data, according to some embodiments. One exemplary configuration of the device is illustrated in FIG. 9A, demonstrating an exemplary interaction between different types of sources (channels) and the processor. Channels representing different biological sources are labeled on the left. In some embodiments, the data from different sources (5A.1, 5A.2, etc.) are recognized by component 5 and undergo different analyses and interpretation. In some embodiments, component 5 can also send commands back to individual sequencers such as stop sequencing, change intensity thresholds, increase or decrease speed, and accept input samples from other channels to increase bandwidth for analysis. FIG. 9B illustrates an example of how asynchronous sequencing of channels can be dynamically assigned to one or more sequencing instruments, according to some embodiments. For example, samples located within the dashed circle can be rotated to different sequencers. In some embodiments, component 5 can signal to component 4 to increase the bandwidth for sequencing channel no. 8 by rotating and distributing its nucleic acids to more sequencers, shown here at the periphery.

FIGS. 10A-10D illustrate exemplary DNA analysis for pathogen detection, estimation of cell quantity, or identification of genetic risk, according to some embodiments. FIG. 10A illustrates an overview of the DNA analysis, species detection, cell number, and genetic risk, from sequencing, according to some embodiments. In FIGS. 10B-10C, a DNA sequence is examined and categorized, e.g., as human or bacteria such as Staph, Strep, E. coli, and viruses, or as reflective of a genetic risk. In scenario 1 illustrated in FIG. 10B, all sequences (SEQ ID NOS 10-14, respectively, in order of appearance) detected belong to Human as illustrated by the filled “tube” and 1M, 1 million counts, a numerical expression of quantity. Scenario 2 illustrated in FIG. 10C depicts pneumonia where a bacterial infection is present; shown in the Strepococcus DNA ‘tube’ are thousands of Strep DNA counts (5 k, 5000). The inset to FIG. 10C describes how the combination of human and Strep DNA counts can be used to provide relative numbers of human vs. Strep cells and organisms. In FIG. 10D, the genetic risk panel describes how the same approach identifies not only species of origin but also human genetic variation, including genetic risk alleles.

FIGS. 11A-11C illustrate exemplary RNA analysis for the identification of affected tissues or patient cellular responses, according to some embodiments. FIG. 11A illustrates an overview of outputs from RNA analysis, tissue of origin and host response, from sequencing, according to some embodiments. In FIGS. 11B-11C, RNA sequence is examined and categorized based on cell of origin such as lung, WBCs, cardiac, RBCs, and non-human bacteria, according to some embodiments. In Scenario 1 (blood) illustrated in FIG. 11B, the most abundant sequences (SEQ ID NOS 10-14, respectively, in order of appearance) detected belong to RBCs and WBCs. A small amount of lung and cardiac RNAs are shown to depict hypothetical normal background of tissue damage. Scenario 2 illustrated in FIG. 11C depicts myocardial infarction, where a cardiac tissue damage is present. Shown in cardiac RNA “tube” is increased numbers of counts (25 k, 25000). The presence of RBCs RNA counts provides an additional assay and quantitative control. Scenario 3 (bacterial pneumonia) illustrated in FIG. 11C demonstrates the combinatorial changes from lung damage, increased immune cells, e.g., WBCs, and bacteria can be identified with a single device, according to some embodiments.

FIGS. 12A-12B illustrate exemplary combined RNA and DNA read counts, according to some embodiments. FIG. 12A illustrates real-time counting of RNA or other measures of gene expression over time demonstrates the increase in detection of specific tissues and cells. In the lower panel of FIG. 12A, a calculation of cell equivalents analyzed thus far provides a physician with the “completeness” of the current results. FIG. 12B illustrates use of genomic DNA as a proxy for cell counts and the integration of data with RNA cell counts. In the pie chart illustrated in FIG. 12B, a proportion of non-human sequences is excluded from calculation of human cell equivalents.

FIGS. 13A-13B illustrate exemplary real-time visualization of RNA and DNA read counts, according to some embodiments. FIGS. 13A and 13B each illustrate a large panel with smaller insets depicting results from four exemplary biological sample sites: blood, urine, sputum, and cerebrospinal fluid (CSF). Within each inset, the expected cell type specific RNA (FIG. 13A) or DNA (FIG. 13B) read count, illustratively in the presence of an infection where both RBCs and a large number of neutrophils are detected in blood.

FIGS. 14A-14B illustrate exemplary interactive results viewing and enlargement for increased detail of cell types tested, according to some embodiments. In FIG. 14A, by selecting the “blood panel” shown in the exemplary interface illustrated in FIG. 13A, the user is offered one or more views to further their understanding where read counts are derived such as shown in FIG. 14B. In the first example illustrated in FIGS. 14A-14B, RNA from RBCs comes predominantly from intact cells, which is a normal phenomenon. In other scenarios, RBC RNA might be abnormally abundant. This result can occur in the setting of hemolytic types of diseases caused by autoimmune or adverse drug events or from poor sampling. In the case of poor sampling, other cell types such as neutrophils are also affected and serve as useful controls for sample quality.

FIGS. 15A-15C respectively illustrate an exemplary selected view of read counts and cells from intact (upper panels) or circulating cell-free (lower panels) samples, according to some embodiments. In FIG. 15A, RNA counts from RBCs and neutrophils are primarily predominantly from intact cells and are representative of cell number. Adjacent to the upper panel of FIG. 15A is a schematic microscopic validation of the instrument's findings. In FIG. 15B, RNA counts from RBCs are abnormally high in circulating cell-free blood. This result is suggestive of hemolysis which can be seen in autoimmune disease, e.g. autoimmune hemolytic anemia; adverse drug events, e.g. drug induced hemolysis; or from poor technical sampling. In this case, poor sampling is unlikely as other cell types such as neutrophils are not affected. In FIG. 15C, poor sample integrity results in cell damage to RBC and neutrophils and can be identified by RNA detection in the circulating cell-free compartment.

FIGS. 16A-16C illustrate exemplary diagnostic report creation, according to some embodiments. In an interaction with the device, the physician can be assisted in creating a results report using data generated by the device and concurrently through the medical record. Exemplary displays of such an interaction are shown in FIGS. 16A-16B with active, possible diagnoses in bold and inactive, excluded diagnoses in italics. Numbers and triangles are used to identify diagnoses with new updated data and to expand current status. In the example shown in FIG. 16A, under the diagnosis of aortic dissection, pending Peripheral BP, completed CXR from the medical record, and device assessment of aortic damage are displayed. DNA percent (%) completion communicates with the physician the completeness of analysis. In the example shown in FIG. 16B, under Acute Myocardial Infarction, inferences drawn from RNA and DNA data are shown as well as pending tests or procedures (Cardiac cath). The physician may also choose to add additional data from the device to support their diagnostic recommendation. The current statistical probability of remaining possible diagnoses is shown in italics. In the example shown in FIG. 16C, symptom characteristics can be correlated to RNA and DNA tests.

FIGS. 17A-17H illustrate examples of how nucleic acid test data can be included in a results summary, according to some embodiments. FIG. 17A illustrates an exemplary likelihood-diagnosis histogram that can be used to display real-time data and mirrors the “early results” and “partial results” respectively shown in FIGS. 17D and 17E. In FIG. 17A, with only 77.5K read counts, the diagnosis is unclear. In FIG. 17B, an adjustable slider can show the trajectory of histogram from one time point (e.g., 77.5K) to another time point (e.g., 900K). In another exemplary display shown in FIG. 17C, a likelihood vs. diagnostic solutions histogram is shown with similar diagnoses grouped. Such a display allows the physician to identify the current status of the device and what diagnoses are being evaluated. For example, the peak labeled as “Myocardial Infarction [MI]” may represent several related diagnoses such as anterior MI, posterior MI, unstable angina, and others or alternatively, independent read count signatures which cumulatively point to MI as the likely diagnosis; in this example, pneumonia is the most likely diagnosis. In FIG. 17D, in an exemplary pneumonia report, the physician chooses to show RNA read counts from blood as supporting evidence for pneumonia. The report also displays other conditions screened and a slider or range window to demonstrate at what stage (and time) was the diagnosis ambiguous and at what point did the diagnosis become well supported. As depicted in FIG. 17D, “early results” of possible diagnoses at 77,500 (77.5K) read counts were still inconclusive, whereas at 510,000 (510K) read counts, the partially complete results suggest a high likelihood of pneumonia and a trajectory that if continued sequencing is performed, the likelihood will continue to improve. Conversely, if the slope of this trajectory has plateaued or is projected to plateau such as shown in FIG. 17E, then the physician would see that continued operation of the device would not improve or change the likelihood of the diagnosis. In FIG. 17F, the physician generates a visual report to support their diagnosis of myocardial infarction. In this example, the physician cites RNA or gene read counts data denoted by i) an arrow, ii) a window of 1M cell equivalents, iii) an icon resembling a magnifying glass to cite the P-value associated with their reference, and other diagnoses considered. FIG. 17G illustrates an exemplary report showing likelihood of a diagnosis as a function of cumulative read counts, and indicating the number of counts estimated to be needed to reach a selected likelihood. FIG. 17H illustrates an exemplary report showing a histogram of the number of read counts for different diagnoses, and indicating the number of counts estimated to be needed to reach a selected likelihood.

FIGS. 18A-18C illustrate an exemplary self-learning process to improve or optimize capture, identification, and interpretation using outcomes data and re-sequencing, according to some embodiments. FIG. 18A illustrates an embodiment in which one or more components from the present devices are designed to be readily exchanged and restored for use. The residual or archived biological samples are a reservoir of useful genetic material that can be used for future optimization of sequence performance, analysis and diagnostic assistance. As illustrated in FIG. 18B, biological samples can be re-sequenced using external sequencing instrumentation to obtain full spectrum of capture vs. non-captured nucleic acids. Longitudinal (patient discharge records) and aggregate outcomes data from other patients are used to improve sensitivity and specificity of device. Improvements can be implemented by modifications in nucleotide targeted sequencing or capture and by modifying datasets to recognize more specific or highly sensitive sequences. FIG. 18C illustrates an example of a sequence of events comparing the output of the device output, re-sequenced samples, and longitudinal and aggregate data to the recognition of sequences with high or low diagnostic value.

FIGS. 19A-19B illustrate an exemplary comparison of longitudinal and aggregate electronic outcomes data to RNA-DNA values, according to some embodiments. FIG. 19A illustrates longitudinal and aggregate (external) data including claims or electronic medical records related to patient and patients are compared to results produced from the device. FIG. 19B illustrates examples of comparison of outcomes between external sources and data produced from device. Inferred values generated from CPT, LOINC, ICD9, ICD10, medications, and RNA-DNA values are tested for matched or mismatched outcomes. In the examples, different tests, inferred values, diagnoses, and treatments are uniquely numbered. Nseq values represent a set of diagnostic sequences, e.g., Nseq4. In the first comparison, inferred values, diagnosis, and treatments match between external and device generated outcomes as indicated by checkmark. The result of this comparison is to add this sample to an aggregate counter for number of matches between device and external data. In the second comparison, there are mismatches between the inferred values, diagnoses and treatments as indicated by “incorrect” checkmark. The result can be recorded for example as a mismatch or decreased matching score between the NSeq24 set of sequences.

FIG. 20 illustrates an exemplary method for use in diagnosing a condition based on a symptom experienced by a subject and based on a first biological sample obtained from the subject, according to some embodiments.

### DETAILED DESCRIPTION

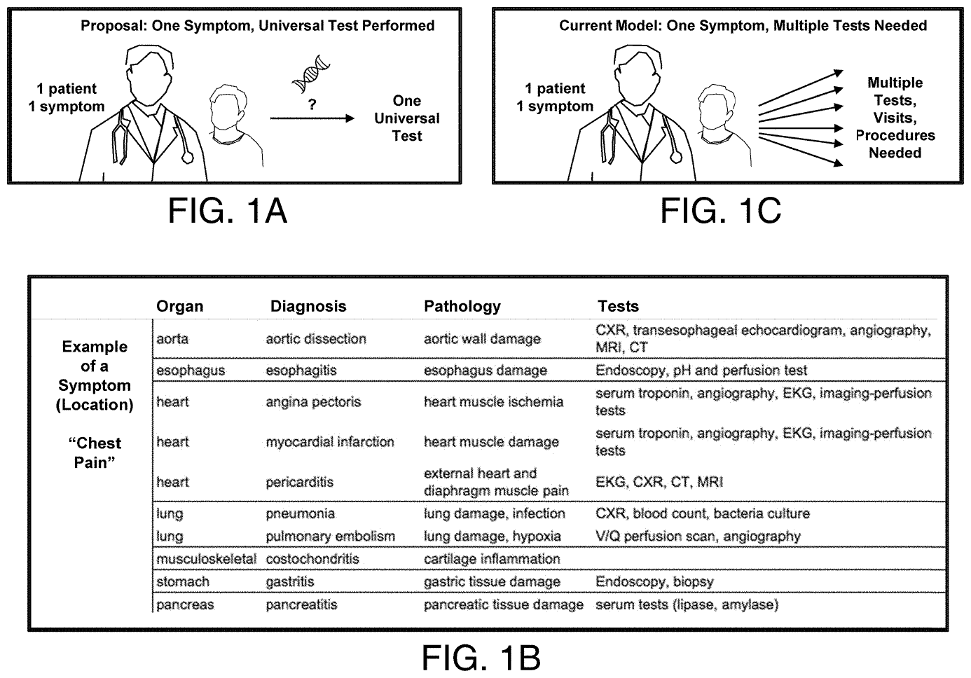

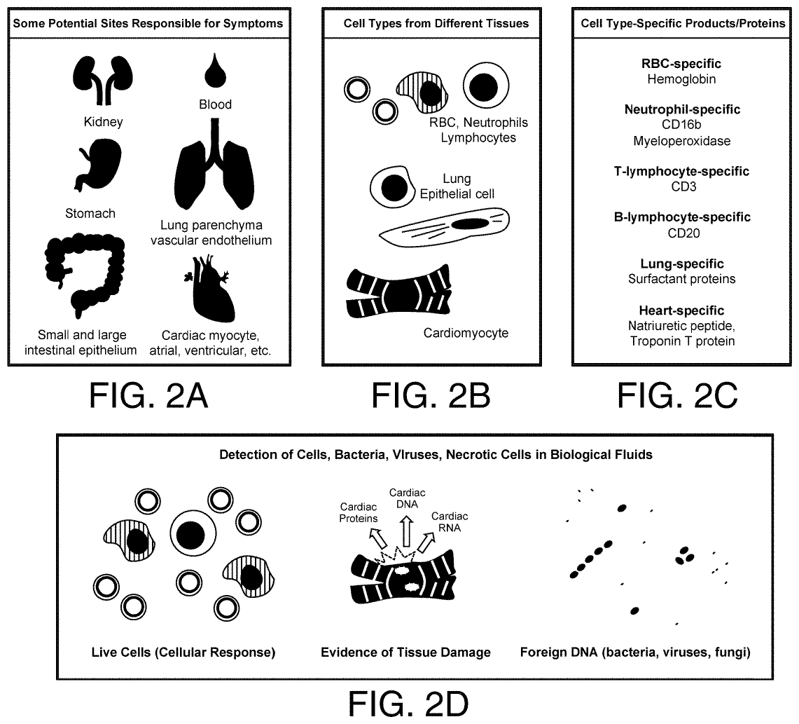

Embodiments of the present invention provide devices and methods for diagnostics based on analysis of nucleic acids. For example, provided herein is a diagnostic device that can simplify and automate nucleic acid testing in the clinic. In certain embodiments, this device can be or include a portable or stationary unit that can facilitate a physician's diagnosis of a cause of patient's symptom, such as an origin or type of tumor or a type of infection, and can provide other competencies, regardless of whether the patient is seen in a community clinic, an emergency room, inpatient hospital, or academic center. Physicians can employ such a device directly to test any suitable number of diagnoses, e.g., tens to hundreds of diagnoses, essentially simultaneously in their patients, without necessarily needing to transmit biological samples offsite. In some embodiments, the device can be configured so as to suitably interact with the physician and to assist in creating results reports that can support or exclude diagnoses, using information generated by the device. For example, FIG. 1C illustrates an exemplary use of nucleic acid-based diagnostics to replace multiple tests, according to some embodiments of the present invention. As used herein, the term “symptom” is intended to mean an abnormal feeling or function experienced by a patient. An identification of a symptom can include some or all of the following: a physiological site of the abnormal feeling or function, a quality of the abnormal feeling or function, a severity of the abnormal feeling or function, a duration of the abnormal feeling or function, a timing of the abnormal feeling or function, a context of the feeling or function, a circumstance under which the abnormal feeling or function can be modified, and any other symptoms that are associated with the abnormal feeling or function.

Overview: How Symptom-Based Testing Reduces Complexity of Nucleic Acid Analysis

Current sequencing devices are not practical for an outpatient setting due to technical and informational complexity. To circumvent these difficulties, certain embodiments of the present devices and methods can provide targeted detection of a limited number of nucleic acid sequences can improve sensitivity, accelerate identification, and interpretation.

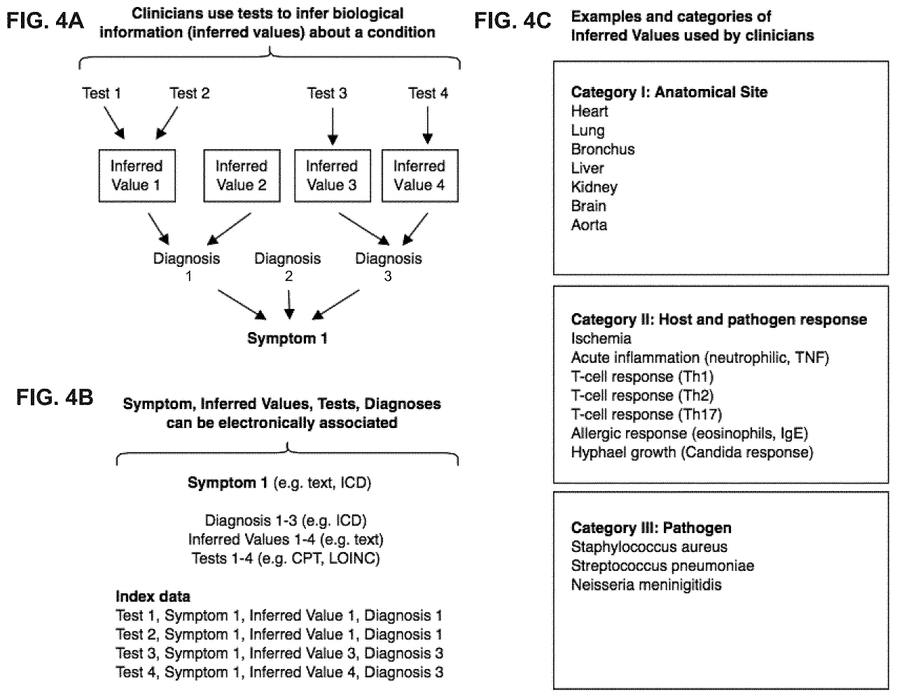

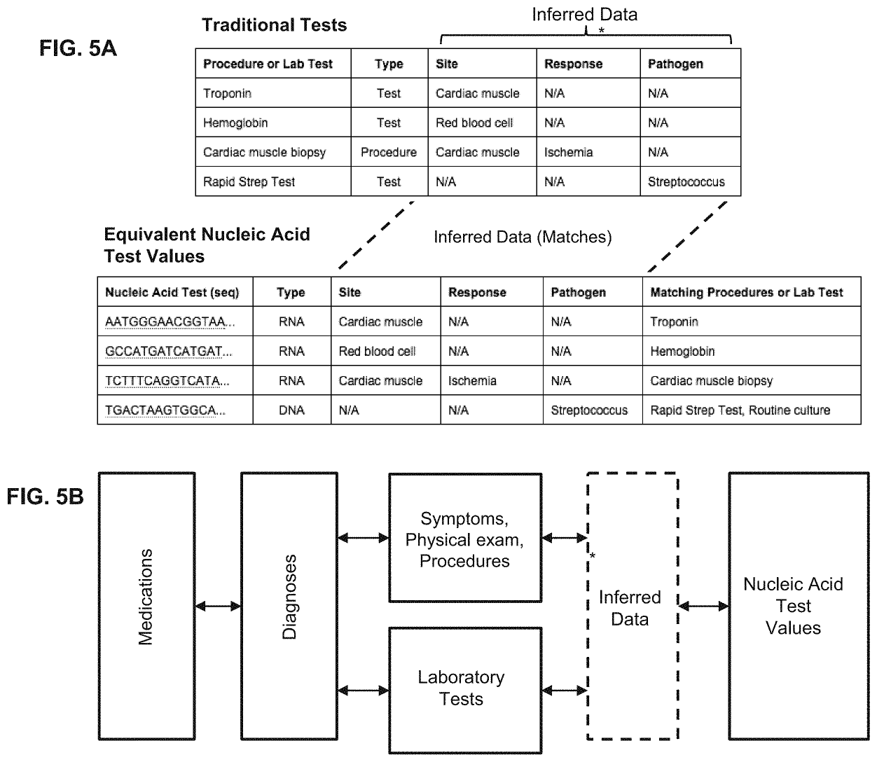

It may not necessarily be known a priori which nucleic acid sequences are clinically informative for a diagnosing particular symptom. Certain embodiments of the present invention provide devices and methods to identify clinically informative sequences. For example, the present devices and methods can generate an external index, hereafter called inferred data. An index of inferred data can be initially created automatically (e.g., by a computer processor executing suitable software) or by humans (e.g., by one or more physician specialists), to define what types of clinical information can be inferred from existing laboratory tests and procedures. For example, FIGS. 4A-4B illustrate relationships between different types of clinical data, according to some embodiments. FIG. 4A illustrates a current diagnostic algorithm used by physicians, in which a combination of tests (e.g., tests 1-4), such as a combination of one or more physical examinations, procedures, or laboratory tests are used to draw inferences (e.g., inferred values 1-4) about the cause (e.g., one or more of diagnoses 1, 2, or 3) of a patient's symptoms (e.g., symptom 1). One or more of the elements illustrated in FIG. 4A can be identified by text or code, e.g., a standard medical code, such as ICD9, ICD10. As illustrated in FIG. 4B, the co-occurrence of diagnoses, procedures, and tests in the same document as a medical claim can link related data. For example, a document can include the patient's symptom (e.g., symptom 1), the potential diagnoses (e.g., diagnosis 1-3), and tests (tests 1-4). Because these tests and procedures generate inferred values, the present devices and methods can prepare an index that includes the patient's symptom (e.g., symptom 1), the potential diagnoses (e.g., diagnosis 1-3), and tests (tests 1-4), as well as inferred data (e.g., inferred values 1-4), based at least in part on such document. FIG. 4C illustrates examples of inferred diagnostic data, organized by categories, and associated values. Such an index can allow for tests and procedures with similar clinical inferences as one another to be recognized and grouped, such as described below with reference to FIGS. 5A-5B. Exemplary categories include anatomical site, host response, or pathogen. In one nonlimiting example, these inferred values include cardiac muscle (from a troponin test), red blood cells and iron status (from a hemoglobin test), presence of Streptococcus (from the Rapid Strep Test), and others. These inferred values can fall under at least three categories: the anatomic site of injury and disease, the patient's cellular response (referred to as host response), and pathogen detection. An index of inferred data can be stored in a suitable computer-readable medium.

In addition to manual approaches to categorize current laboratory test and procedures, automated approaches can be employed using medical literature and internet. Electronic search for individual test and procedure names can identify the context of a test within a chapter or data in various text formats such as HyperText Markup Language (html). For example, in one nonlimiting example in the html format, “<B>Troponin</B>” and “Cardiac” may appear adjacent to one another or within quotes in a given document. In one approach, a frequency is generated by counting the number of times individual laboratory tests and procedures appear together with a list of clinically relevant terms representing anatomic site, pathogen, or host response. A non-limiting example of this approach is shown here:

```
get@(url, term, secondaryArray) // e.g. getTermsCount(
{
  htmlDomDocument = getDomDoc(url)
  // get the html data from the url
  numOccurrences =
  getOccurrencesNumberOfTerms(htmlDomDocument,term)
  // get the number that a term occurs within the document
  output = initArray(output,term,secondaryArray)
  // set all values in a 2D array to 0.
  // output[term][secondaryArray[0]]=0, output[term][secondaryArray[1]]=0
  for(i=0;i<numOccurrences;i++) // for each occurence
  {
  elementWithSearchTerm =
  htmlDomDocument.findContainingElement(term,i)
    // get the html element containing the next term
  foreach(secondaryArray as secondary) // for each secondary term
  {
    if(getOccurrencesNumberOfTerms(elementWithSeearchTerm,secondary)
    >0)
    //if it occurs more than one time
    {
    output[term][secondary] = output[term][secondary]+1
    // increment the output
    }
  }
  }
  display(output) // display the results
}
```

A second automated approach performs text-based search among public and private medical records such as described above with reference to FIG. 4B. These documents can include codes or text representing the laboratory tests, procedures and diagnoses used to evaluate individual patients. The grouping of diagnoses, laboratory tests and procedures within a single claim or encounter document link together related elements. For example, the diagnosis of acute myocardial infarction can be identified as text or by the prefix (410) in the medical code called International Classification of Disease (ICD). Significantly, the grouping of these diagnoses, tests and procedures can represent a diagnostic workflow in identifying the cause of a patient's symptoms and disease. Illustratively, the output of these processes can include an index of a test name and a frequency of its association with one or more inferred value categories, which output suitably can be stored in a computer-readable medium.

As described in greater detail below with reference to FIGS. 5A-5B, inferred data values can also be related to nucleic acid test data. For example, inferred data values (e.g., heart) can be used as search terms to identify and associate nucleic acid test values. Illustratively, search can be performed on public sequence databases such as National Center for Biotechnology Information (NCBI), ENCODE (NIH), Sequence Read Archive (SRA), European Bioinformatics Institute (EMBL) and others, and can returns nucleic acid test values in the form of or in documents containing nucleic acid sequences (e.g., an actual sequence, or in the form of FASTA, FASTQ, or other formats), gene names, species genome and their quantity, e.g., frequency or rank of a specific gene among many genes detected. Using the identified gene names and species genome as search terms, related nucleic acid sequence values can be derived from public databases. Another source of nucleic acid test data can be derived within the laboratory, where nucleic acids can be extracted from different cell types, tissues, and pathogens using common molecular biology methods and reagents and sequenced (further explained below). The result of this process is the electronic storage of nucleic acid sequences (e.g. AATGGGAACGGTAA (SEQ ID NO: 4) in FIG. 5B, described in greater detail below) and association with inferred data values (e.g. heart, Streptococcus, etc.). This data in turn creates relationships between an inferred data value and multiple related tests and nucleic acid values.

For example, FIGS. 5A-5B illustrate exemplary generation of clinically informative sequences through common inferred data, according to some embodiments. FIG. 5A illustrates a diagnostic algorithm that utilizes physical exams, procedures, and laboratory tests to draw inferences about the cause of a patient's symptoms. Inferred data (dashed box) can be categorized, for example, as an anatomical location of disease (site), a cellular response (resp.), microorganism or pathogen (micro) detection. Similar information, or the same information, can be derived based on values from nucleic acid analysis such as described in greater detail herein.

Illustratively, a database can be stored in a computer-readable medium, the database storing at least a plurality of symptoms, a nucleic acid sequence associated with each of the symptoms, a potential diagnosis associated with each of the symptoms, a laboratory test or a procedure for each of the symptoms, and an inferred value for each of the symptoms, the inferred value comprising a clinical inference based on a result of said laboratory test for the respective symptom, e.g., such as described herein with reference to FIGS. 4A-5B.

In some embodiments, a method of generating a database stored in a computer-readable medium can include receiving, by a device (e.g., by a suitably programmed processor), a plurality of medical documents. For example, the device can include a computer-readable medium (which can be the same as or different than the computer-readable medium in which the database is stored) in which the plurality of medical documents can be stored. Each document can describe at least one symptom experienced by a respective patient, a laboratory test or a procedure performed on that patient, and a diagnosis associated with the at least one symptom experienced by that patient, the diagnosis being based on a result of the laboratory test performed on that patient. The method also can include, by the device, inferring values based on the symptoms, the laboratory tests, and the diagnoses described in the plurality of medical documents, each inferred value comprising a clinical inference based on a result of at least one of the laboratory tests for the respective symptom, e.g., such as described herein with reference to FIGS. 4A-5B. The method also can include, by the device, identifying a nucleic acid test value associated with each of the inferred values. The method also can include, by the device, generating and storing in the computer-readable medium a plurality of database entries, each database entry of the plurality comprising a symptom, a laboratory test or a procedure performed on a patient having that symptom, at least one possible diagnosis associated with that symptom, an inferred value for that diagnosis, and a nucleic acid test value for that inferred value. The database can be queried or updated such as described elsewhere herein.

Optionally, the nucleic acid test value can include an RNA sequence or a DNA sequence. Additionally, or alternatively, the nucleic acid test values can include one or more specific nucleic acid sequences, one or more groups of nucleic acid sequences, one or more quantities of nucleic acid sequences, one or more patterns of nucleic acid sequences, or one or more contexts of nucleic acid sequences. Illustratively, the one or more contexts of nucleic acid sequences can include one or more associations of nucleic acid sequences with chemical modifications, proteins, other intramolecular or extramolecular nucleic acids, or intracellular or extracellular subcompartments. The plurality of medical documents can include standard medical codes describing at least some of the symptoms, laboratory tests or procedures, and diagnoses. Additionally, or alternatively, the plurality of medical documents further can include physical findings, medications, or environmental exposures.

FIG. 5B illustrates examples of how four procedures or laboratory tests can be linked to specific nucleic acid test sequences based on inferred data. For example, beginning with the relational index discussed above with reference to FIG. 4B, a search for a given symptom can return a group of associated diagnoses, tests and inferred values. The same search can return a group of nucleic acid test values, which represent the same group of clinical information. For example, in FIG. 5B, a search for the symptom “chest pain” can retrieve several diagnoses, including myocardial infarction. Along with the diagnosis (e.g., myocardial infarction), inferred values (e.g., “cardiac muscle” and “heart”) are also returned. The term “cardiac muscle” also appears in the nucleic acid test values, “AATGGGAACGGTAA” (SEQ ID NO: 4) and “TCTTTCAGGTCATA” (SEQ ID NO: 5) and thus, two sequences can be related to the symptom, chest pain.

In some embodiments, nucleic acid values (e.g., “AATGGGAACGGTAA” (SEQ ID NO: 4)) can be further evaluated for their uniqueness. For example, the nucleic acid value (“AATGGGAACGGTAA” (SEQ ID NO: 4)) can be found both in brain and heart tissue. A symptom-based approach can be used to determine if distinguishing between these two tissues may be necessary or useful in a given clinical scenario. For example, in the presence of the symptom ‘chest pain’, the detection of circulating “AATGGGAACGGTAA” (SEQ ID NO: 4) is more likely to be representative of damaged heart tissue than damaged brain tissue. As indicated above, specificity of nucleic acid sequences is likely to vary depending on the sample site, e.g., blood versus urine. Additional methods can be incorporated into the identification of specific patterns or quantities of nucleic acid sequences, use of quantitative differences, threshold cut-offs, rank orders, and other means. The output of this process is a set of nucleic acid sequences, which are non-overlapping with nucleic acid sequences present in competing diagnoses.

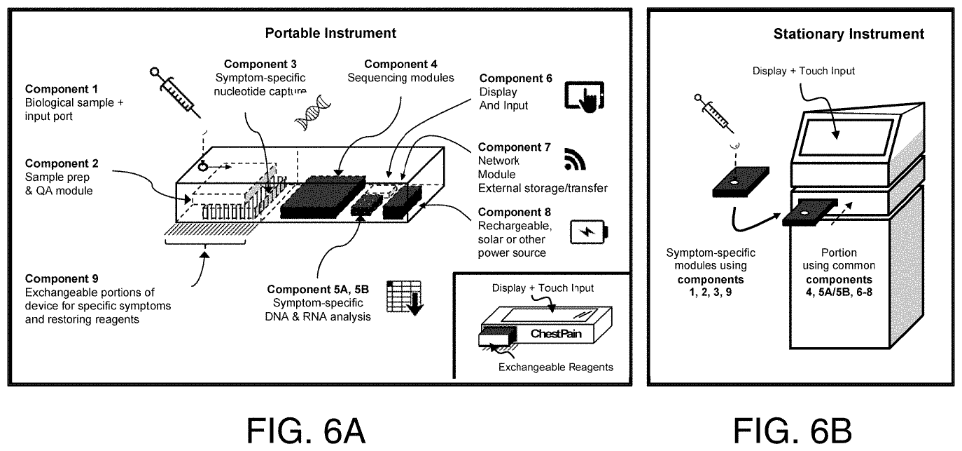

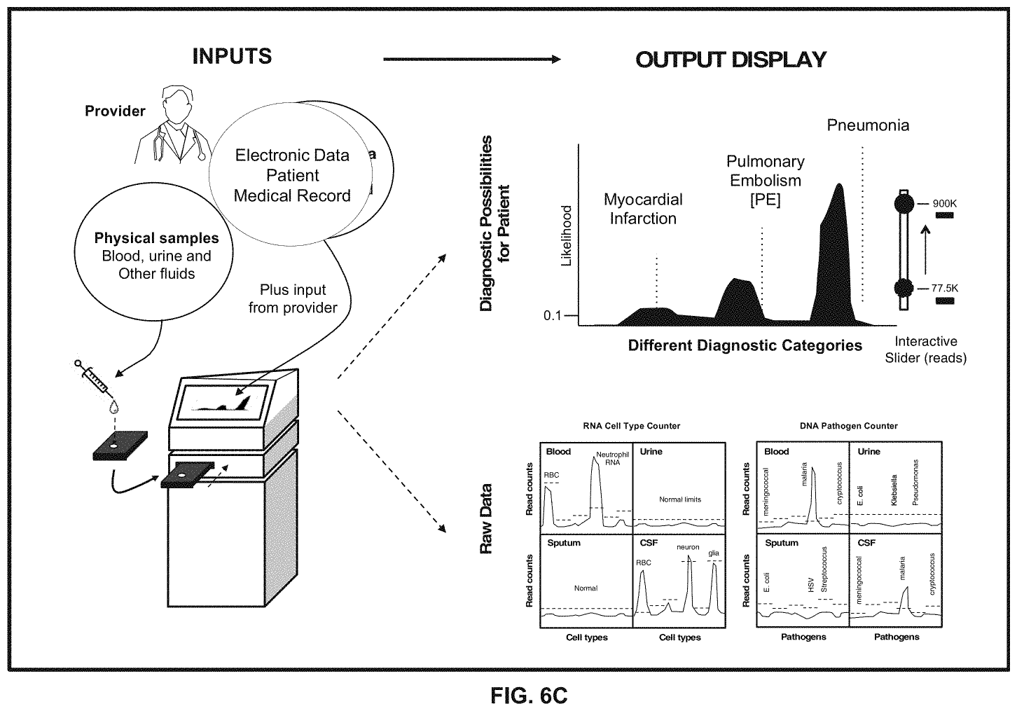

Illustratively, a method for performing one or more nucleic acid tests based on one or more symptoms experienced by a patient can include receiving by a device (e.g., by the instruments described herein with reference to FIGS. 6A-9B), respective identifiers of the one or more symptoms experienced by the patient. The method also can include, by the device, submitting to a database a query based on the respective identifiers of each of the one or more symptoms. The database can include a computer-readable medium storing at least a plurality of symptoms, a nucleic acid sequence associated with each of the symptoms, a potential diagnosis associated with each of the symptoms, a laboratory test or a procedure for each of the symptoms, and inferred data for each of the symptoms, the inferred value comprising a clinical inference based on a result of said laboratory test for the respective symptom. Non-limiting examples of methods of generating such a database are provided elsewhere herein. The method also can include, by the device, receiving from the database a response to the query, the response comprising one or more nucleic acid tests based on the nucleic acid sequences respectively associated with the one or more symptoms identified in the query. The method also can include, by the device, outputting respective representations of the one or more nucleic acid tests. The method also can include receiving, by a receptacle of the device, a cartridge configured to perform at least one of the one or more nucleic acid tests.

In some embodiments, the method further includes, by the device, outputting a result of the at least one of the one or more nucleic acid tests, the result comprising a count of RNA or DNA of the subject or of a pathogen in the subject, the RNA or DNA having the nucleic acid sequence associated with at least one of the one or more symptoms identified in the query. Additionally, or alternatively, the response to the query can include a representation of a plurality of nucleic acid tests based on a plurality of nucleic acid sequences respectively associated with the one or more symptoms identified in the query, the cartridge being configured to perform each nucleic acid test of the plurality. Additionally, or alternatively, the method further can include receiving, by a receptacle of the device, at least one additional cartridge, the at least one additional cartridge being configured to perform at least one other of the nucleic acid tests.

Under another aspect, a device (e.g., an instrument such as described herein with reference to FIGS. 6A-9B) for performing one or more nucleic acid tests based on one or more symptoms experienced by a patient includes an input module configured to receive respective identifiers of the one or more symptoms experienced by the patient (e.g., input component 6 described herein with reference to FIGS. 6A-9B). The device also can include a query module configured to submit to a database a query comprising the respective identifiers of each of the one or more symptoms. The database can include a computer-readable medium storing at least a plurality of symptoms, a nucleic acid sequence associated with each of the symptoms, a potential diagnosis associated with each of the symptoms, a laboratory test or a procedure for each of the symptoms, and inferred data for each of the symptoms, the inferred value comprising a clinical inference based on a result of said laboratory test for the respective symptom. For example, component 5A-5B of the device can include such a query module that is configured to access the database (which optionally can be remote) via component 7. The query module further can be configured to receive from the database a response to the query, the response comprising one or more nucleic acid tests based on the nucleic acid sequences respectively associated with the one or more symptoms identified in the query. The device further can include an output module configured to output respective representations of the one or more nucleic acid tests. For example, the device can include display component 6 configured to display such output to a caregiver, or can include a computer-readable medium to which the output may be recorded, or can include a communication module, e.g., component 7, via which the device can provide the output to another computer or another computer-readable medium. The device further can include receptacle configured to receive a cartridge configured to perform at least one of the one or more nucleic acid tests, e.g., a receptacle for receiving one or more symptom-specific modules 9.

In some embodiments, the output module optionally further is configured to output a result of the at least one of the one or more nucleic acid tests, the result comprising a count of RNA or DNA of the subject or of a pathogen in the subject, the RNA or DNA having the nucleic acid sequence associated with at least one of the one or more symptoms identified in the query. Additionally, or alternatively, the response to the query can include a representation of plurality of nucleic acid tests based on a plurality of nucleic acid sequences respectively associated with the one or more symptoms identified in the query, the cartridge being configured to perform each nucleic acid test of the plurality. Additionally, or alternatively, the receptacle of the device further can be configured to receive least one additional cartridge, the at least one additional cartridge being configured to perform at least one other of the nucleic acid tests.

The above described exemplary approach highlights that the scope of nucleic acid testing and interpretation can be greatly narrowed based on a method of inferred data values and symptom-specific data structure. Furthermore, such a symptom-based methodology can address several major obstacles in nucleic acid testing and can facilitate the miniaturization and improvement of a nucleic acid diagnostic instrument.

Overview of Diagnostic Device and Exemplary Use Thereof

An overview of an exemplary diagnostic device as a portable or stationary unit to detect active genes and foreign DNA to test multiple diagnoses, according to some embodiments, is illustrated in FIGS. 6A-6C. FIG. 6A illustrates an exemplary portable configuration, and FIG. 6B illustrates an exemplary stationary configuration. The components illustrated in FIGS. 6A and 6B can allow or facilitate automated sample preparation, sequencing (e.g., nucleic acid sequencing), and clinical interpretation. Component 1 can be configured so as to receive different types of biological samples (e.g., fluids). Illustratively, component 1 includes an input port for receiving a biological sample, such as from a syringe. Component 2 can be fluidically coupled to component 1 and can be configured so as to prepare and enrich biological specimens, e.g., to prepare and enrich biological sample(s) received by component 1. Illustratively, component 2 can include a sample preparation and quality assurance (QA) module. Component 3 can be fluidically coupled to component 2 and can be configured so as to perform sequence-specific capture, e.g., symptom-specific nucleotide capture. For example, component 3 can include a first set of complementary nucleic acids configured to capture a first set of nucleic acids, the first set of nucleic acids being selected based on the symptom, which can be defined by one or more characteristics of symptoms. The first set of complementary nucleic acids can capture a first plurality of nucleic acids of the first set that are present in a first biological sample from a subject experiencing the symptom. Component 4 can be fluidically coupled to component 3 and can be configured to perform sequencing, e.g., can include one or more sequencing modules. For example, component 4 can include a nucleic acid sequencer configured to sequence each captured nucleic acid that is present in the first biological sample. Optionally, component 4 also can include a separator configured to separate extracellular nucleic acids in the first biological sample from intracellular nucleic acids in the first biological sample. Optionally, the nucleic acid quantifier and nucleic acid sequencer can separately operate on the separated extracellular nucleic acids and on the intracellular nucleic acids.

Component 5 can be electronically coupled to component 4 and can be configured so as to perform sequence identification and quantification, e.g., so as to perform symptom-specific DNA (component 5A), RNA (component 5B), and integrated analyses (component 5C). Illustratively, component 5 (also referred to herein as 5A/5B/5C) can include a processor and one or more computer-readable media storing instructions to cause the processor to perform one or more of the functions provided herein, and also storing information for use in performing nucleic acid analysis. For example, component 5 can include a nucleic acid quantifier configured to quantify an amount of each of the captured nucleic acids that is present in the first biological sample. For example, component 5 can include a processor coupled to the quantifier and to the sequencer and that is suitably programmed to identify an origin of each captured nucleic acid based on the sequence of that captured nucleic acid. In embodiments that include a separator, the processor can be suitably programmed to cause the display component 6 or other output module to output an indication of the quantified amount of at least one of the extracellular nucleic acids and an indication of the quantified amount of at least one of the intracellular nucleic acids. Note that the terms “component” and “module” can be used interchangeably herein.

Optionally, the device can include a computer-readable medium coupled to the processor, and the processor further can be suitably programmed to identify the origin of the captured nucleic acid based on comparing the sequence of that nucleic acid to sequences stored in a library stored in the computer-readable medium. Optionally, the library stores nucleic acid sequences for a human and for a plurality of pathogens. The output from the device can indicate the relative number of a pathogen per human cell.

Component 6 can be electronically coupled to components 5, 7, 8 and can be configured so as to interact with the user, e.g., can include display and input components. For example, component 6 can include a display that is coupled to the processor, and the processor (e.g., of component 5) can be suitably programmed to cause the display to output an indication of the quantified amount and the identified origin of at least one captured nucleic acid that is present in the first biological sample. Optionally, the processor can be suitably programmed to cause the display to output an indication of the quantified amount of each of the captured nucleic acids of the first plurality. As another example, the processor (e.g., of component 5) can be suitably programmed to cause the display to output an indication of at least one potential diagnosis for the subject and an indication of the likelihood of the at least one diagnosis based on the quantified amount and the identified origin of at least one captured nucleic acid that is present in the first biological sample.

Component 7 can be electronically coupled to components 5 and 8 and configured so as to provide connectivity to electronic medical records, a database such as described elsewhere herein, and external sequence analysis, e.g., can include a network module. Component 8 can be coupled to components 1-7 and 9 and configured to provide a power source, e.g., can include a rechargeable, solar, or other power source, or can be configured so as to connect to a standard AC power outlet. Component 9 includes portions of the device that can be exchanged, allowing for optimization, customization, or restoration. For example, component 9 can include exchangeable portions of the device for specific symptoms, and restoring reagents, such as some or all of components 1, 2, and 3. For example, the inset to FIG. 6A is intended to illustrate an exemplary embodiment in which display and input (e.g., a combined display and touch input) can be provided on the top of the device, as well as an exemplary location of exchangeable reagents. In the exemplary stationary configuration illustrated in FIG. 6B, display and input (e.g., display and touch input) can be provided in a readily accessible portion of the device, e.g., on a front panel; a portion using common components 4, 5A/5B, and 6-8 can be provided in the device; and symptom-specific modules using components 1, 2, 3, and 9 can be inserted into the device. In some embodiments, the device illustrated in FIG. 6A or the device illustrated in FIG. 6B are configured to receive a first set of complementary nucleic acids within a first symptom-specific cartridge, e.g., within component 9. The first symptom-specific cartridge can be removable and replaceable with a second symptom-specific cartridge including a second set of complementary nucleic acids. Optionally, the first set of complementary nucleic acids is different than the second set of complementary nucleic acids. That is, the devices can be configured to receive different types of cartridges that are specific to different symptoms and that include different sets of complementary nucleic acids than one another.

In some embodiments, the first set of complementary nucleic acids (e.g., of component 3) further captures a second plurality of nucleic acids of the first set that are present in a second biological sample obtained from the subject, the second biological sample being different from the first biological sample. The nucleic acid quantifier (e.g., of component 5) further can quantify an amount of each of the captured nucleic acids that is present in the second biological sample. The nucleic acid sequencer (e.g., of component 4) further can sequence each of the captured nucleic acids that is present in the second biological sample. The processor (e.g., of component 5) further can be suitably programmed so as to identify an origin of each captured nucleic acid based on the sequence of the captured nucleic acid that is present in the second biological sample. The processor further can be suitably programmed so as to cause the display (e.g., of component 7) to output the an indication of quantified amount and the identified origin of at least one captured nucleic acid that is present in the second biological sample.

Illustratively, to activate the device, the healthcare worker can input a physical identification or a touch code via Component 6 (FIG. 6C). Illustratively, a display screen of Component 6 can also report which diagnoses the device is best suited for identifying and excluding and what types of biological input samples are used in the analysis. In some embodiments, responsive to the device being activated, the device can be digitally paired between the worker and the patient using a medical record number or other patient identifying information. In some embodiments, pairing can activate a wireless data transfer of the patient medical record to the device via Component 7. The past medical history can be used by Component 5 of the device to prioritize, to modify diagnostic possibilities and to provide treatment recommendations, based on an on-board dataset rules or remotely from an off-site facility.

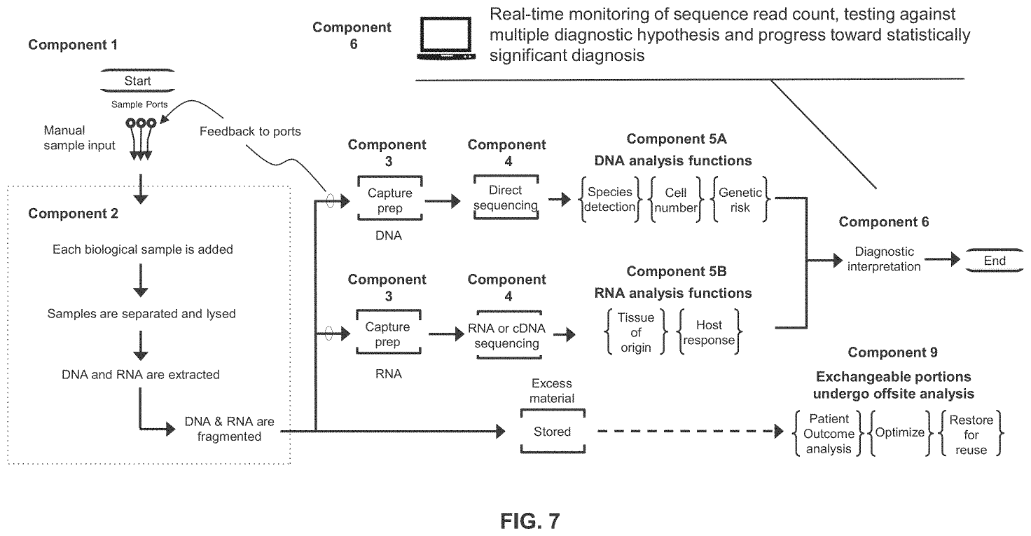

Further exemplary details of the components illustrated in FIGS. 6A-6B are provided below. For example, FIG. 7 illustrates a detailed overview of exemplary internal components involved in sample preparation and analysis, according to some embodiment. Component 1 can be configured to receive different types of sample inputs, e.g., fluidic samples, e.g., blood, urine, or the like, and in some embodiments can be configured to receive feedback from DNA or RNA sensors. Component 2 can be configured to separate and count intact cells, extracellular particles, and liquids; to lyse cells; to extract nucleic acids; or to clean and fragment DNA and RNA; or to perform any suitable combination of the foregoing. Components 3 and 4 can be configured to quantify DNA and RNA (oval) received from component 2; to provide feedback to Component 1 (e.g., can include DNA or RNA sensors); to capture DNA or RNA; or to perform targeted sequencing; or to perform any suitable combination of the foregoing. Component 5 (5A/5B) respectively can be configured to perform DNA and RNA analysis by comparing test data to a pre-computed index of sequences representing species, cell types, and host responses that can be relevant to the patient's symptoms. In addition, component 5 can be configured so as to quantify and normalize results. Component 6 can include an electronic interface configured to facilitate real-time monitoring of results; for network- and geographical location updating; or for interactions with interactions with the physician, including assistance with diagnostic interpretation; or any suitable combination of the foregoing. Component 9 can include exchangeable parts of the device which, in some embodiments, can undergo offsite re-sequencing of remaining material, aggregation of patient outcome data from the device and from the medical record; optimization of results interpretation through analysis of concordant and discordant outcomes; or capacity to modify reagents in components 2, 3, 5, and 6 for improved accuracy and sensitivity.

Component 1: Preparing Samples

In some embodiments, during use, the user can inject or deliver one or more biological samples, e.g., via a syringe, capillary tube, or pipette, into Component 1, which can include one or more sample ports of the device, such as illustrated in FIG. 7. In some embodiments, the ports are configured so as to receive pressurized samples (e.g., positive-pressure from syringe injection) or so as to passively uptake liquids. Component 1 then transmits the biological sample(s) to Component 2 (see below). The ports in Component 1 can also provide a feedback indicator to the user. For example, based on nucleic acid sensors in Component 3, an electronic signal can be returned to Component 1 to provide the user with suitable feedback, including whether the device is ready to receive samples, which port is ready (e.g., continuous white light), status of sample, or which port requires more sample (e.g., colored light).

Component 2: Separating Samples and Preparing Nucleic Acids

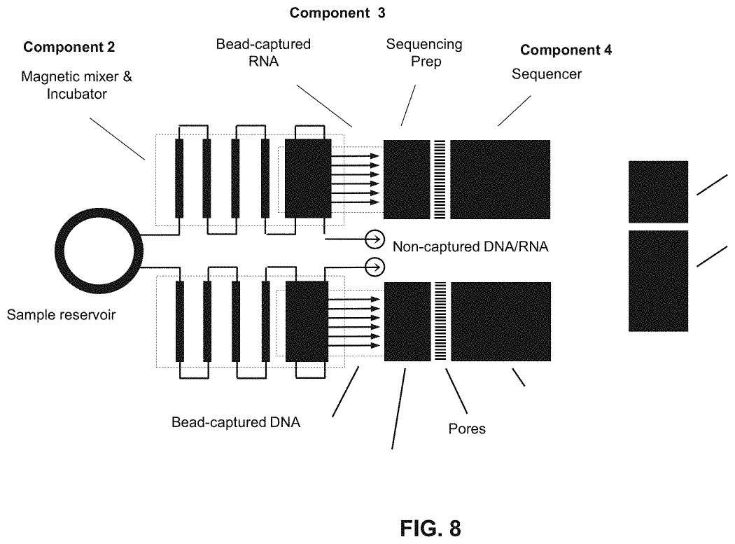

The process of receiving biological liquids, separation, lysis and nucleic acid processing can be achieved by adjoined components from existing methodologies and devices. For example, FIG. 8 illustrates an exemplary physical layout of components used to receive and sequence biological samples, according to some embodiments. For example, FIG. 8 illustrates an embodiment in which adjoining Components 2, 3, 4, and 5 are configured so as to cooperatively process samples and nucleic acids and to analyze the results of such processing. In Component 2, biological specimens are stored in sample reservoirs and enter into microfluidic chambers by passive, negative or positive pressures, or other means. Within channels, e.g., varying caliber channels, cells and particles can be separated or affinity captured. DNA and RNA are released as cells encounter chambers with lytic agents and localized heat or vibration. In one nonlimiting embodiment, Component 2 includes a sample reservoir that is fluidically coupled to a magnetic mixer and incubator. In Component 3, free DNA and RNA molecules are selectively captured (e.g., bead-captured RNA/DNA) and moved to an area involved in sequencing prep reactions, e.g., ligation of adaptors. These select nucleic acids can be sequenced by Component 4, which can include a nucleic acid sequencer, such as commercially available, or other nucleic acid detection instrument. Component 5 can include a processor, such as an internal computer, e.g., sequence detection and data integration processor, that is suitably programmed so as to receive electronic data from Component 4 and to determine the identity of and counts of detected nucleic acids, e.g., using an internal sequence lookup database suitably stored on a computer-readable medium, optionally that can be updated periodically, or an external database.

In one nonlimiting example, sample reception, storage and subsequent separation (Component 2) can be achieved using known or customized microfluidic components, such as microfluidic ChipShop spiral or pillar particle and cell sorting chips (e.g., part numbers #18-1708-0382-01 or #19-1800-0261-01, commercially available from microfluidic ChipShop, Jena, Germany), micro-droplet separator, or other separation approaches. In some embodiments, compartments may be initially separated but can become open based on an electronic signal from a controller (e.g., Component 5) or natively in response to the presence of liquids. The separation of samples based on size, visual or other properties can allow the device to identify whether the analyte, e.g., DNA or RNA, originated from an intact cell or debris from a damaged cell. In some embodiments, following this stage, the analytes, e.g., DNA and RNA, can be present within liquid solution as a complex with cellular proteins or as free molecules. Further purification of protein-associated nucleic acids and enrichment of free nucleic acids can be performed, using well-known methods of immuno-purification of DNA such as commercially available Clontech EpiXplore ChIP assay kits (Clontech Laboratories, Inc., Mountain View, Calif.). Additionally, isolation of DNA and RNA can be performed using existing methods and reagents, such as Bioneer Accuprep (Bioneer Corporation, Daej eon, Korea), Qiagen AllPrep DNA/RNA FFPE Kit (Qiagen Inc., Valencia, Calif.), or Zymo Research ZR-Duet (Zymo Research Corporation, Irvine, Calif.) and electrical, heat or mechanical methods, such as ChipGenie (microfluidic ChipShop, Jena, Germany) to disrupt cells, release and isolate DNA and RNA.

Component 3 and 4: Selection of Clinically Relevant DNA and RNA Molecules to Assay

From the total population of DNA and RNA molecules, clinically informative sequences can be enriched using established methods, such as nucleotide capture (e.g. Agilent SureSelect (Agilent Technologies, Santa Clara, Calif.), Nextera Rapid Capture (Illumina, Inc., San Diego, Calif.), or the like) or targeted sequences using oligonucleotides. To select specific sequences, publicly available computational approaches such as the Wessim Whole Exome Sequencing SIMulator using in silico exome capture (a Python based simulator available for download from sak042.github.io/Wessim) can be used to predict and simulate capture oligonucleotides. Using such an approach, the device can target a select group of sequences that collectively facilitate distinguishing between multiple diagnostic possibilities for each clinical scenario. The types of sequences captured can depend on the clinical context and can be pre-defined by symptoms as discussed above with reference to FIGS. 5A-5B.

In some embodiments, component 4 can include known components to perform the sequencing of nucleic acids. Indeed, there are many ways to identify specific sequences. In one example, selected nucleic acids from Component 3 can be prepared for sequencing using methods well-known to molecular biologists, and can be sequenced using one or more known devices, such as Illumina Mi-Seq (Illumina, Inc., San Diego, Calif.), Life Technologies Ion Torrent (Life Technologies, Thermo Fisher Scientific Inc., Waltham, Mass.), or the like. In brief, such devices use DNA from the patient sample as a template to make new copies of DNA. This copy process can be monitored chemically or visually and is used to record the order in which individual nucleotides are added into the new copy of DNA. This order corresponds to the sequence of the DNA. Such sequence can be compared to sequences in a database, and the order of nucleotides in the sequence can reveal the identity and origin of the sequence, e.g., human, bacteria, fungus, virus, and subtypes of species, and individual specific differences, e.g., drug-resistance and states. Sequencing instruments (currently known as next generation sequencers) can allow for relatively large numbers of nucleic acids to be sequenced in parallel. This attribute can facilitate testing multiple molecules simultaneously and flexibility in their application.

In particular embodiments, the methods of the invention can be performed with next generation sequencing (NGS) using commercially available kits and instruments from companies such as the Life Technologies/Ion Torrent PGM or Proton (Life Technologies, Thermo Fisher Scientific Inc., Waltham, Mass.), the Illumina HiSEQ or MiSEQ (Illumina, Inc., San Diego, Calif.), and the Roche/454 next generation sequencing system (Roche Diagnostics Corporation, Basel, Switzerland). NGS technology is rapidly revolutionizing the fields of genomics molecular diagnostics, and personalized medicine through the increasingly efficient and economical generation of unprecedented volumes of data. See, e.g., the following references, the entire contents of each of which is incorporated by reference herein: Didelot et al., “Transforming clinical microbiology with bacterial genome sequencing,” Nature Rev. Genetics, 13: 601-612 (2012); Biesecker et al., “Next generation sequencing in the clinic: Are we ready?” Nature Rev. Genetics 13: 818-824 (2012); Martin et al., “Next-generation transcriptome assembly,” Nature Rev. Genetics 12: 671-682 (2011); Voelkerding et al., “Next-generation sequencing: From basic research to diagnostics,” Clin. Chem. 55: 641-658 (2009); Su et al., “Next-generation sequencing and its applications in molecular diagnostics,” Expert Rev. Mol. Diagn. 11: 333-343 (2011); Meyerson et al., “Advances in understanding cancer genomes through second-generation sequencing,” Nature Rev. Genetics 11: 685-696 (2010); and Zhang et al., “The impact of next-generation sequencing on genomics,” Journal of Genetics and Genomics=Yi chuan xue bao 38: 95-109 (2011).

Some commonly used NGS platforms are the 454 GS Junior (Roche Diagnostics Corporation, Basel, Switzerland), Ion Torrent (Life Technologies, Thermo Fisher Scientific Inc., Waltham, Mass.), and MiSeq (Illumina, Inc., San Diego, Calif.), which are “benchtop” sequencers designed for laboratory use. These platforms are capable of a wide range of sequencing applications due to their versatility in sample type, experiment scale, instrument protocol, and multiplexing options. See, for example, the following references, the entire contents of which are incorporated by reference herein: Liu et al., “Comparison of next-generation sequencing systems,” J. Biomedicine & Biotechnology, 2012: Article ID 251364, 11 pages (2012); Loman et al., “Performance comparison of benchtop high-throughput sequencing platforms,” Nature Biotechnol. 30: 434-439 (2012); Glenn, “Field guide to next-generation DNA sequencers,” Mol. Ecol. Resources 11: 759-769 (2011); and Quail et al., “A tale of three next generation sequencing platforms: comparison of Ion Torrent, Pacific Biosciences and Illumina MiSeq sequencers,” BMC Genomics 13: 341, 13 pages (2012). The 454 and Ion Torrent platforms use emulsion PCR to generate millions of DNA molecules with the same sequence from a single sample molecule attached to a polymer bead. The Illumina platforms use bridge PCR to amplify single surface-bound molecules to generate a cluster of molecules with the same sequence. Templates are then sequenced by a stepwise incorporation of nucleotides (e.g., Illumina Genome Analyzer, Roche Applied Science 454 Genome Sequencer) or short oligonucleotides (e.g., Applied Biosystems SOLID (Applied Biosystems, Thermo Fisher Scientific Inc., Waltham, Mass.)). Both the bridge PCR and emulsion PCR methods of parallel amplification require the ligation of adapter sequences to the ends of sample DNA molecules to create sequencing libraries that can bind to surface or bead-bound probes complementary to the adapters.

In addition, the analysis of nucleic acids can be performed using any technique known in the art including, without limitation, sequence analysis, and electrophoretic analysis. Non-limiting examples of sequence analysis include Maxam-Gilbert sequencing; Sanger sequencing; capillary array DNA sequencing; thermal cycle sequencing such as disclosed in Sears et al., “CircumVent thermal cycle sequencing and alternative manual and automated DNA sequencing protocols using the highly thermostable VentR (exo-) DNA polymerase,” Biotechniques, 13: 626-633 (1992), the entire contents of which are incorporated by reference herein; solid-phase sequencing such as disclosed in Zimmerman et al., “Fully automated Sanger sequencing protocol for double stranded DNA,” Methods Mol. Cell Biol., 3: 39-42 (1992), the entire contents of which are incorporated by reference herein; sequencing with mass spectrometry such as matrix-assisted laser desorption/ionization time-of-flight mass spectrometry (MALDI-TOF/MS) such as disclosed in Fu et al., “Sequencing exons 5 to 8 of the p53 gene by MALDI-TOF mass spectrometry,” Nat. Biotechnol, 16: 381-384 (1998), the entire contents of which are incorporated by reference herein; and sequencing by hybridization, such as disclosed in the following references, the entire contents of each of which are incorporated by reference herein: Chee et al., “Accessing genetic information with high-density DNA arrays,” Science, 274: 610-614 (1996); Drmanac et al., “DNA sequence determination by hybridization: a strategy for efficient large-scale sequencing,” Science, 260: 1649-1652 (1993); and Drmanac et al., “Accurate sequencing by hybridization for DNA diagnostics and individual genomics,” Nat. Biotechnol., 16:54-58 (1998). Non-limiting examples of electrophoretic analysis include slab gel electrophoresis such as agarose or polyacrylamide gel electrophoresis, capillary electrophoresis, and denaturing gradient gel electrophoresis.

As for the identification of specific DNA sequences, detection of active genes also can be based on highly specific RNA sequences. A target mRNA can be amplified by reverse transcribing the mRNA into cDNA, and then performing PCR (reverse transcription-PCR or RT-PCR). The reverse transcription step is typically primed using specific primers, random hexamers, or oligo-dT primers, depending on the circumstances and the goal of expression profiling. For example, extracted RNA can be reverse-transcribed using a GeneAmp® RNA PCR kit (Applied Biosystems, Thermo Fisher Scientific Inc., Waltham, Mass.) according to the manufacturer's instructions. The derived cDNA can then be used as a template in a subsequent PCR reaction.

RNA-seq is an emerging technology for surveying gene expression and transcriptome content by directly sequencing the mRNA molecules in a sample. RNA-seq can provide gene expression measurements and is regarded as an attractive approach to analyze a transcriptome in an unbiased and comprehensive manner.

Various methods for determining expression of mRNA, protein, or gene amplification include, but are not limited to, gene expression profiling, polymerase chain reaction (PCR) including quantitative real time PCR (qRT-PCR), RNA-Seq, FISH, microarray analysis, serial analysis of gene expression (SAGE), MassARRAY, proteomics, and immunohistochemistry (IHC).

Histone modifications have been implicated in the regulation of gene expression and genome function. Chromatin Immunoprecipitation followed by hybridization (Chip-on-CHIP) and Chip-sequencing (ChIP-Seq) can be used to determine the localization of this modification at specific genomic locations and to determine which genes are targeted and turned on and off in a variety of diseases and disorders.

Gene expression profiles can be readily obtained by any number of methods known in the art, for example, microarray analysis, individual gene or RNA screening (e.g., by PCR or real time PCR), diagnostic panels, mini chips, NanoString chips (nanoString Technologies, Seattle, Wash.), RNA-seq chips, protein chips, or ELISA tests.

Methods of measuring a level of a polypeptide gene product are known in the art and include assays that utilize a capture agent. In some embodiments, the capture agent is an antibody, antibody fragment, nucleic acid-based protein binding reagent, small molecule or variant thereof. In additional embodiments, the assay is an enzyme immunoassay (EIA), enzyme-linked immunosorbent assay (ELISA), and radioimmunoassay (RIA). In some embodiments, detection and/or quantification of one or more biomarkers further comprises mass spectrometry (MS). In yet further embodiments, the mass spectrometry is co-immunoprecitipation-mass spectrometry (co-IP MS), where coimmunoprecipitation, a technique suitable for the isolation of whole protein complexes is followed by mass spectrometric analysis.

Component 5: Analysis of Sequence Data from Multiple Sources

In some embodiments, component 5 can include a suitably programmed processor, which, responsive to instructions on a computer-readable medium, takes as input data from Component 4 (the sequencing instrument) and compares such data to a pre-computed and stored library of sequences such as described in greater detail above with reference to FIGS. 5A-5B. In some embodiments, Component 5 can be configured so as to continuously read data from component 4 that includes the order of nucleotides (“AAATATAGAATATGTATTGCGG . . . ” (SEQ ID NO: 6)), or can be configured so as to interpret intermediate output from component 4, such as the most recent nucleotide detection results (“A”). Other exemplary intermediate outputs include nucleotide base calls, raw images, conductivity measures, and other outputs from available sequencing instruments, such as Illumina Mi-Seq, Ion Torrent, and others. For intermediate outputs, component 5 can store data in memory along with fragment or location ID data to build a sequence of nucleotide information which can be performed using instrument software, e.g., Illumina CASAVA (Illumina, Inc., San Diego, Calif.), Off-line Base Caller (Illumina, Inc., San Diego, Calif.), AYB (All Your Base) base caller from the European Bioinformatics Institute (Cambridge, United Kingdom), or the like. Simple computational methods allow for the identification of exact sequence matches (e.g. UNIX ‘grep’ command) while the most probable match when the sequence is not exact can be identified using Smith-Waterman algorithm, Burrows-Wheeler transform, and the like.

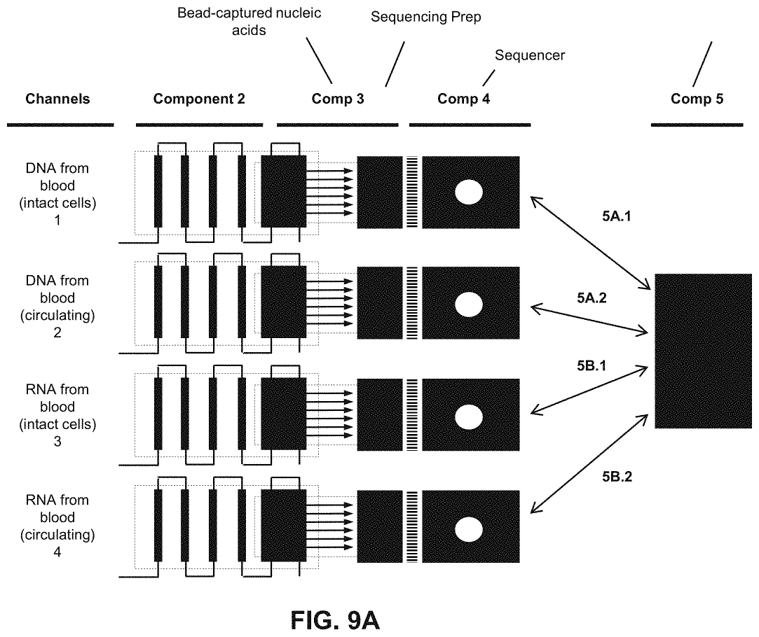

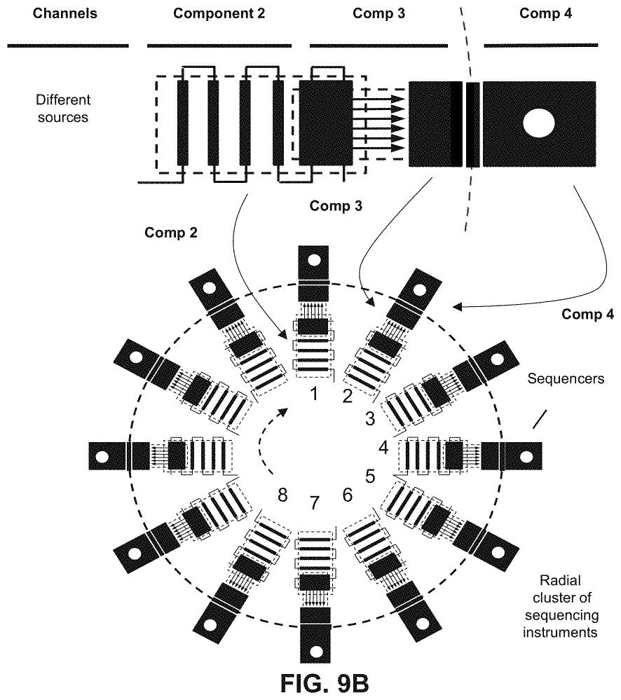

As discussed above, nucleic acid information is obtained from multiple sources such as different sample sites (e.g., blood, urine, CSF), RNA vs. DNA, intact cells, circulating cell debris, association with specific molecules (e.g. modified histones) and others. Component 5 is able to recognize these different sources of nucleic acid data. In FIGS. 9A-9B, one exemplary configuration of this process utilizes multiple sequencing instruments, which can each be dedicated to analyzing one source of nucleic acids and separately transfer their output to component 5 for analysis. FIG. 9A describes an exemplary manner in which Component 5 can be configured so as to send commands to the sequencer, Component 4, or to upstream components. Some commands may signal for the reaction or sequencer to ‘stop’. Other commands can modify the detection of nucleotides by the sequencer including changing intensity thresholds for one or more nucleotide detection, altering flow rates of material to individual sequencers, or assigning specific or the number of sequencing devices to assaying a biological source. In one example, when sequencing of one biological sample (e.g., urine) is already complete, the now available sequencer can be reassigned to share analysis of a second biological sample (e.g., blood).

A non-limiting example of this synchronization of multiple samples across multiple nucleic acid testing devices is shown below:

```
  Let n be the number of sequencers. Given n samples, assign each
  sample a set of diagnoses and initialize its likelihood to 0.
  class sample {
    likelihood = 0
    diagnoses = [ ]
  }
// the above instruction resets the result derived likelihood and expected
diagnosis likelihood threshold to 0 until the expected diagnosis likelihood
is provided to the device.
  def computeRank(samples):
    ranks = [ ]
    for each sample in samples:
      rank = (sample.likelihood / threshold(sample.diagnoses)) *
      (1 / impact(sample.diagnoses))
      ranks.append(rank)
    return ranks
// the above instruction ranks the samples based on the progress of the
analysis and a weighting factor to reflect prioritization by clinical impact.
  def run(sequencers, samples):
    nFinished = 0;
    for each sample in samples:
    assignSequencer(sample)
    while(n != nFinished):
    for each sample in samples:
      sample. likelihood = computeLikelihood(sample, sample.diagnoses)
// the above instruction assigns likelihood to each sample.
  for each sample in samples:
    if sample.likelihood > threshold(sample.diagnoses):
    archive(sample)
    makeAvailable(sequencer)
    samples -= sample
    nFinished++
// the above instruction checks to see if a sample is complete, as defined
by the sample likelihood surpassing a completion threshold specific to
that sample. Upon completion, the sample is archived and the sequencer
is now made available.
  if(isAvailableSequencer( )):
    sample =min(computeRank(samples))
    assignSequencer(sample)
// the above instruction checks to see if a sequencer is available. If so,
samples with the appropriate Rank are distributed to additional available
sequencers.
```

One exemplary arrangement for accommodating asynchronous sequencing of a biological source across multiple sequencers is depicted in FIG. 9B, where in a radial configuration, nucleic acids in channel 4 are analyzed in two or more sequencers shown at the periphery. Another exemplary arrangement similar to the radial model described in FIG. 9B is a spiral or helical arrangement of sequencers and channels, which could accommodate increasing numbers of samples and sequencers.

Illustratively, a method for use in diagnosing a condition based on a symptom experienced by a subject and based on a biological sample obtained from the subject, the biological sample including nucleic acids, the method being executed by a device (such as an instrument described herein with reference to FIGS. 6A-9B), can include, over a first period of time, quantifying by the device an amount of a first subset of the nucleic acids that are present in the biological sample, the first subset of the nucleic acids having a first origin. The method also can include, over the first period of time, quantifying by the device an amount of a second subset of the nucleic acids that are present in the biological sample, the second subset of the nucleic acids having a second origin that is different than the first origin. The method also can include outputting by the device an indication of the amount of the first subset of the nucleic acids quantified over the first period of time. The method also can include outputting by the device an indication of the amount of the second subset of the nucleic acids quantified over the first period of time.

Optionally, the method can include, based on the amount of the first subset of the nucleic acids quantified over the first period of time, ceasing quantifying by the device an amount of the first subset of the nucleic acids over a second period of time that is subsequent to the first period of time. The method also can include, based on the ceasing, over the second period of time, quantifying by the device an amount of a third subset of the nucleic acids that are present in the biological sample, the third subset of the nucleic acids having a third origin that is different than the first origin and that is different than the second origin. The method also can include outputting by the device an indication of the amount of the third subset of the nucleic acids quantified over the second period of time.

Optionally, the device can include a sequencer that quantifies the first subset of the nucleic acids over the first period of time and that is reassigned so as to quantify the third subset of the nucleic acids over the second period of time. Additionally, or alternatively, the ceasing is based on an estimation by the device of a first likelihood that the subject is suffering from a first condition, the estimation being based on the amount of the first subset of the nucleic acids quantified over the first period of time. Optionally, the ceasing further can be based on a comparison by the device of the estimation to a threshold.

Under another aspect, a device (e.g., an instrument such as described herein with reference to FIGS. 6A-9B) for use in diagnosing a condition based on a symptom experienced by a subject and based on a biological sample obtained from the subject, the biological sample including nucleic acids, can include a first quantification module configured to quantify, over a first period of time, an amount of a first subset of the nucleic acids that are present in the biological sample, the first subset of the nucleic acids having a first origin, e.g., components 5A-5B. The device also can include a second quantification module configured to quantify, over the first period of time, an amount of a second subset of the nucleic acids that are present in the biological sample, the second subset of the nucleic acids having a second origin that is different than the first origin, e.g., components 5A-5B. The device also can include an output module configured to: output an indication of the amount of the first subset of the nucleic acids quantified over the first period of time, and to output an indication of the amount of the second subset of the nucleic acids quantified over the first period of time, e.g., a display component 6 configured to display such indications, or a computer-readable medium configured to store such indications, or component 7 configured to transmit such indications to a computer.

Optionally, the first quantification module is configured to cease, based on the amount of the first subset of the nucleic acids quantified over the first period of time, quantifying an amount of the first subset of the nucleic acids over a second period of time that is subsequent to the first period of time. The first quantification module can be configured to quantify, based on the ceasing, over the second period of time, an amount of a third subset of the nucleic acids that are present in the biological sample, the third subset of the nucleic acids having a third origin that is different than the first origin and that is different than the second origin. The output module further can be configured to output an indication of the amount of the third subset of the nucleic acids quantified over the second period of time. Additionally, or alternatively, the first quantification module can include a sequencer that quantifies the first subset of the nucleic acids over the first period of time and that is reassigned so as to quantify the third subset of the nucleic acids over the second period of time. Additionally, or alternatively, the ceasing can be based on an estimation by the device of a first likelihood that the subject is suffering from a first condition, the estimation being based on the amount of the first subset of the nucleic acids quantified over the first period of time. Additionally, or alternatively, the ceasing further can be based on a comparison by the device of the estimation to a threshold.

Component 5A: Sequence Analysis of DNA in Detecting Pathogens and Humans

In particular embodiments, the present devices and methods can be employed to detect a viral infection, a Gram positive bacterial infection or a Gram negative bacterial infection, a parasite or a fungus.

Component 5 uses data such as described above for several types of analyses, including species identification, estimation of cell and microbe number, disease predisposition, personalization of safe treatment options, detection of tissue and cell type damage, determine host cellular responses, identify pathogen response, determine which treatments the pathogens are sensitive to, and others. Below is a description of an exemplary manner in which the device analyzes and utilizes information from different nucleic acid analytes.

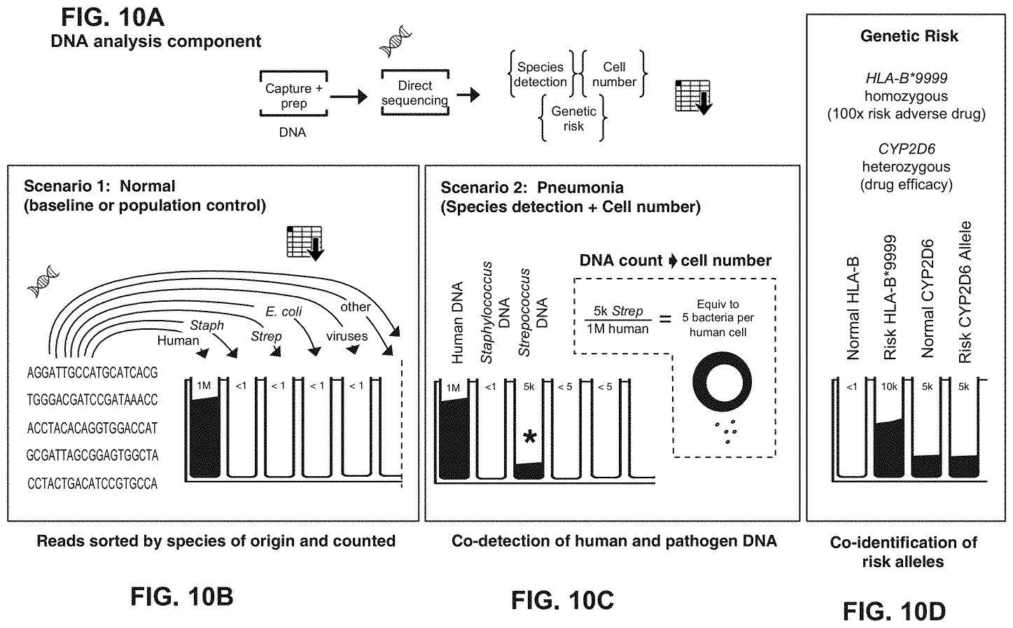

For DNA matches, any of several analyses can be performed to output specific types of clinical data to the physician. This DNA analysis process are described as component 5A, which can include a suitable processor, associated memory and database (which may be the same as component 5), which, responsive to instructions on a computer-readable medium, takes as input electronic data from the sequencer (Component 4) to analyze and output to the physician which genes or biomarkers are detected via Component 6. FIGS. 10A-10D illustrate exemplary DNA analysis for pathogen detection, estimation of cell quantity, or identification of genetic risk. FIG. 10A provides an overview of the DNA analysis, species, detection, cell number, and genetic risk, based on the results of genetic sequencing. Component 5A counts the number of multiple non-human species and human DNA molecules (defined as DNA read counts) observed. In this process, specific sets of human and non-human DNA sequences are pre-selected using methods described in more detail in FIGS. 5A-5B. An exemplary sequence TGACTAAGTGGCA (SEQ ID NO: 7) is stored and related to its origin, a type of bacteria, Streptococcus. This sequence and additional Streptococcus sequences are pre-loaded into a database contained within component 5 and associated with information such as the bacteria identity and which specimens the sequences should be screened in. These channels and sequences can be pre-selected for species that require screening in the context of the sample type, e.g., urine, blood, and the like, and symptom. Some sequences are selected for their non-unique characteristics and serve as a common denominator to quantify the total number of sequences analyzed (see below and in Component 6). In FIGS. 10B and 10C, the DNA sequence is examined and categorized as human or foreign, e.g., bacteria, such as Staphylococcus, Streptococcus, E. coli, or a particular virus. In hypothetical “normal” (baseline or population control) Scenario 1 illustrated in the FIG. 10B, sequences of DNA in a sample are compared to sequences of selected organisms, and are categorized based on the comparison. For example, in the hypothetical Scenario 1 illustrated in FIG. 10B, all detected sequences are determined to be human, as suggested by the filled “tube.” Additionally, the quantity of human DNA can be expressed as “counts;” in the illustrated example, 1 million (1 M) counts of human DNA were observed.

Hypothetical “pneumonia” (species detection and cell number) Scenario 2 illustrated in FIG. 10C depicts pneumonia where a bacterial infection is present. In Scenario 2, in the setting of an infection (pneumonia), data from a blood sample matches to multiple human DNA sequences (e.g., 1 million read counts) in addition to 5,000 read counts of a bacterial DNA (e.g. Streptococcus). The inset illustrates how the combination of human and Strep DNA counts can be used to provide relative numbers of human versus Strep cells and organism. In this hypothetical example, it is shown that 1 million human DNA read counts is equivalent to 1,000 human cells, based on the in vitro results in the lab. This pre-computed threshold can be used to communicate the relative number of bacteria per human cell to the physician. This latter result would be unexpected in a normal person and raise concerns for a physician.

DNA analysis can also reveal patient genetic markers. Genetic markers are a type of variation in a patient's DNA sequence that is correlated with disease risk, an indication for a medication, or adverse drug event risk. For example, FIG. 10D illustrates how an analogous approach as applied in Scenarios 1 and 2 can be used to identify not only a species of origin but also human genetic variation, including genetic risk alleles. Such findings can be “incidental,” e.g., not the intent of the genetic test, but depending on the type of genetic risk, may be required to report to the patient and doctor. If the patient already has sequence data available from previous tests or previous use of the device, genetic markers can additionally be used to confirm the identity of patient. Through DNA analysis process, additional types of guidance, e.g. genetic risk, treatment modification, and patient identity, can be generated. In the hypothetical example illustrated in FIG. 10D, the patient can be determined to have a genetic risk based on the detection of homozygous HLA-B*9999 genotype, depicted by 10,000 counts of the HLA-B*9999 allele and <1 counts of the normal HLA-B allele. In this hypothetical example, the patient is also identified as heterozygous for a CYP2D6 risk allele, depicted by 5,000 counts of both the normal CYP2D6 and risk CYP2D6 alleles.

Component 5B: Detects Affected Cells and Host Responses by Gene Detection

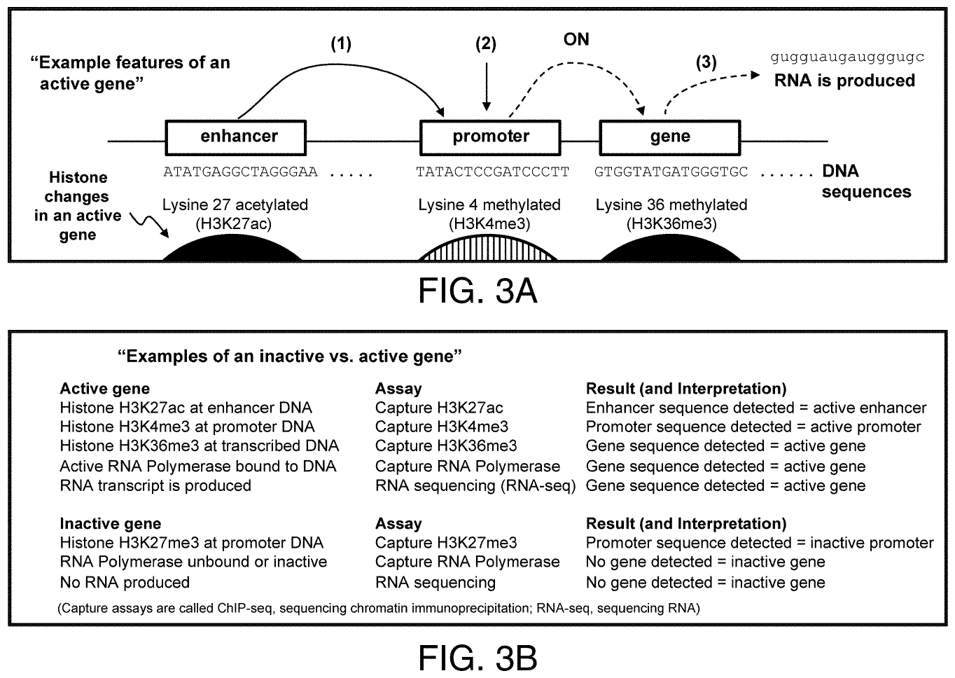

In some embodiments, component 5B can include a suitably programmed processor (which may be the same as the processor of component 5), which, responsive to instructions on a computer-readable medium, takes as input electronic data from the sequencer (Component 4) to analyze and output to the physician which genes or biomarkers are detected via Component 6. For example, component 5B can use electronic data to detect products of activated genes either from intact cells or from circulating cell debris, and uses this information to derive two major functions: which cells are present and what types of host responses are occurring. In some embodiments, gene detection, e.g., RNA or chromatin immunoprecipitation of DNA, can be used to determine which tissues are damaged and what the cellular responses are present. As described above, e.g., with reference to FIGS. 3A-3B, many different cell types can be identified by which genes they turn on.

Additionally, existing approaches align data to an encyclopedia of sequences representing one or more genomes. The location of where this alignment occurs within the encyclopedia can be used to obtain additional information. Although common, such a practice is in some ways analogous to scanning through 3 billion pages for a match and is computationally complex. In some embodiments, the present methods and devices can use a more targeted approach, such as so called targeted sequencing and alignment-free method, so as to as to scan through significantly fewer total number of possible sequences, potentially saving significant time and improving sensitivity. For further details regarding alignment free methods, see, e.g., Vinga et al., “Alignment-free sequence comparison-a review,” Bioinformatics, 19: 513-523 (2003), the entire contents of which are incorporated by reference herein.

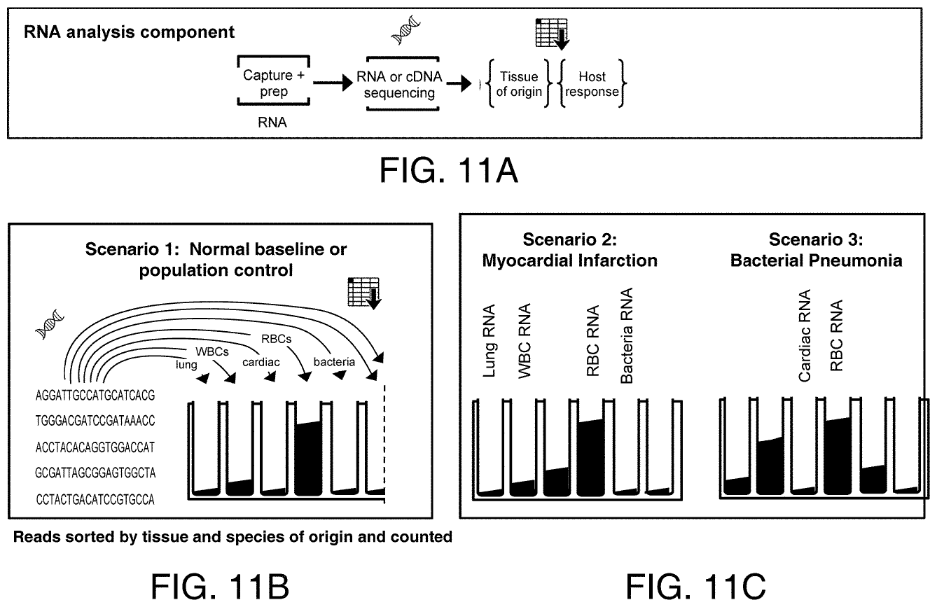

FIGS. 11A-11C illustrate exemplary RNA analysis for the identification of affected tissues or patient cellular responses, according to some embodiments. FIG. 11A illustrates an overview of outputs from RNA analysis, tissue of origin, and host response, from sequencing. In hypothetical “normal baseline or population control” Scenario 1 illustrated in FIG. 11B, RNA sequences from a sample are examined and categorized, based on comparison of the RNA sequence to sequences in a database. Such categorization can be, for example, according to cell of origin, such as lung, white blood cell (WBC), cardiac, RBC, or non-human bacteria. In FIG. 11B, Scenario 1, some sequences reflect genes only activated in the lung, while other active genes can only be found in white blood cells. In this exemplary Scenario 1 (biological sample is blood), the most abundant sequences detected belong to RBCs and WBCs. A relatively small amount of lung and cardiac RNAs are shown to depict hypothetical, exemplary, normal background of tissue damage. In the hypothetical example illustrated in Scenario 1, the device can detect RNA sequences representative of blood cell RNAs, and the detection of these RNA sequences is expected and confirmatory as red blood cells present in blood. On the other hand, the detection of large amounts of RBCs or WBCs in the urine or cerebrospinal fluid would be highly concerning for infection or trauma. Thus, interpretation of results can depend on the sample site.

Hypothetical “myocardial infarction” Scenario 2 illustrated in the left panel of FIG. 11C depicts exemplary counts of detected RNA from different cellular sources in the presence of cardiac tissue damage. The “tubes” illustrated for Scenario 2 correspond to the “tubes” for Scenario 1. For example, in Scenario 2, in the setting of cardiac damage, the device would detect RNAs produced from heart cells released from damaged heart tissue, in addition to similar counts of RNAs as in Scenario 1. For example, in Scenario 2, the cardiac RNA “tube” includes an increased or elevated count of cardiac RNAs (e.g., 25,000 in this hypothetical example). The presence of RBC RNA counts can provide an additional assay and quantitative control. Hypothetical “bacterial pneumonia” Scenario 3 illustrated in the right panel of FIG. 11C depicts exemplary counts of detected RNA from different cellular sources in the presence of bacterial pneumonia. In an infection such as pneumonia, Scenario 3, the device would detect both the presence of damaged lung cells, the RNAs from increased immune cells and RNA and DNA from bacteria. Hypothetical scenario 3 (bacterial pneumonia) demonstrates exemplary combinatorial changes from lung damage, increased immune cells, e.g., WBCs, and bacteria that can be identified with the present device.

Detecting Host Response

In some embodiments, in addition to identifying which cells are present, the present device can be configured so as to report how cells respond to disease, infection, and changes in the environment. Physicians frequently order microscopic exam of blood, urine, and other biological fluids and other tests to identify the presence or absence of specific populations of immune cells. For example, in bacterial infections, acute inflammatory cells, e.g. neutrophils, when detected in the blood, urine or wound site are a sensitive indicator for a possible infection. In parasitic and drug hypersensitivity responses, “allergic”-type responses can be seen, e.g., increased eosinophils (eosinophilia). In viral responses, increased numbers of lymphocytes and different immune states are often noted. In other examples, the presence of different RBC stages, e.g., normoblasts or reticulocytes, is used as evidence of high turnover and hematopoietic disturbances. Thus, use of RNA counts allows the physician to the detection or shifts in the abundance of immune cells.

Other tests of host responses do not involve a shift in cell number but can be detected by RNA changes. For example, ferritin and transferrin receptor levels are sensitive assays to diagnose systemic low iron conditions. Response to low iron arises from activation of genes, whose products are used in iron transport and absorption. Other types of cell states can represent chronic injury, hypoxia, and hyperactivation. Thus, potentially important clinical information can be found in addition to the identification and quantity of a cell.

Component 5C: Integration of RNA and DNA Data

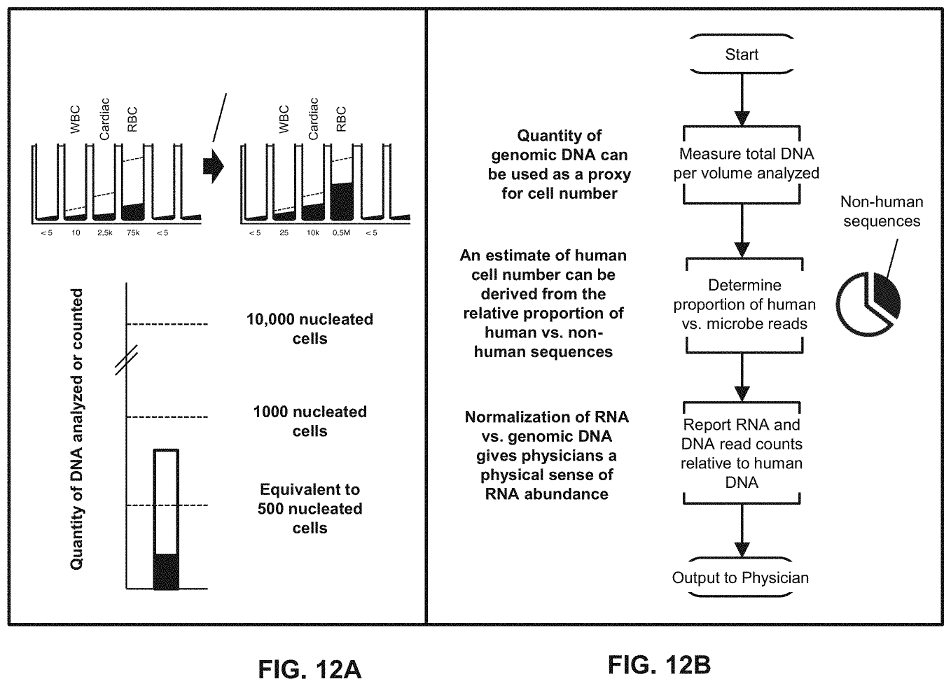

Component 5C can include a suitably programmed processor (which may be the same as the processor of component 5), which, responsive to instructions on a computer-readable medium, takes as input electronic data from DNA and RNA analysis in Component 5A and 5B to analyze and output to the physician via Component 6. In FIGS. 12A-12B, an example is provided to demonstrate how data from component 5A and 5B can be integrated. In some embodiments, the device reports current cell number as a means to communicate a conceptual measure of time or relative completeness (FIG. 12A). Cell number can be derived directly through counting cells prior to lysis or, alternatively, estimated through DNA quantities or other stable quantities of molecules, such as ribosomes or histones. DNA content and other stable molecules are essentially fixed during the majority of a cell's life (>95% of a human cell's lifespan) and thus serve as an indirect measure of cell quantity. The interpretation of RNA in the context of DNA or cell number is critical as this information can be used to convey how relevant or probable a test result means. For example, a relatively small number of cells analyzed can reflect a relatively low sample number, whereas a relatively higher cell number can reflect relatively higher sample number. Thus, in some embodiments, higher cell numbers improve the confidence that a result is reproducible and increase the likelihood that even rare events have been screened and excluded.

Real-time results of RNA data from Component 5B are then coupled to the current DNA data from Component 5A. In the embodiment illustrated in the upper panel of FIG. 12A, an output can be displayed, representing real-time counting of RNA or other measures of gene expression over time demonstrates the increase in detection of specific tissues and cells. In the embodiment illustrated in the lower panel of FIG. 12A, a calculation of cell equivalents analyzed thus far can provide the physician with a measure of the “completeness” of the current results such as described in greater detail above.

For example, FIG. 12B illustrates an exemplary process flow that integrates the use of genomic DNA as a proxy for cell counts and RNA cell counts calculated from Component 5A and displayed via Component 6. In the pie chart illustrated in FIG. 12B, in one exemplary embodiment, a proportion of non-human sequences is excluded from calculation of human cell equivalents. The device can use the relative amount of human vs. non-human DNA read counts and the total DNA per volume to more accurately estimate human or microbe cell number. The process illustrated in FIG. 12B can thus report RNA read counts relative to human DNA and provide the physician a physical sense of RNA abundance. Other embodiments integrate RNA and DNA read counts to estimate the number or proportion of specific cell types relative to other cell types.

Embodiments of the invention can encompass both qualitative and quantitative detection of a nucleic acid in a biological sample. In this regard, qualitative detection can be useful, for example, for recognizing an infection of an individual. Thereby, one aspect is that false-negative or false-positive results be avoided. In addition to mere detection of the presence or absence of a nucleic acid in a sample, it-can be useful to determine the quantity of said nucleic acid. As an example, stage and severity of a viral disease may be assessed on the basis of the viral load. Further, monitoring of any therapy can use information on the quantity of a pathogen present in an individual in order to evaluate the therapy's success. For a quantitative assay, a quantitative standard nucleic acid can serve as a reference for determining the absolute quantity of a nucleic acid.

Component 6: Result Reporting and Real-Time Requirements

Component 6 includes, or operates as, an input-output interface such as a digital touch-screen and computer. Component 6 can be configured to output visual and interactive representations of data from Components 5A, 5B, and 5C. The description that follows provide exemplary types of visual outputs and interactions with the physician supported by the device. In addition to the real-time analytical reports described with reference to FIGS. 12A-12B, the device can supports the physician's ability to interpret and report RNA and DNA data in a diagnostic workflow, in some embodiments. The interaction between the device and physician can assist in identifying clinically significant results and follow familiar diagnostic workflows. In some embodiments, these interactions can further serve to quantify and learn how physicians prioritize specific RNA and DNA sequences to support or exclude a diagnosis. The following describe some types of result categories, which are reported by the device in some embodiments.

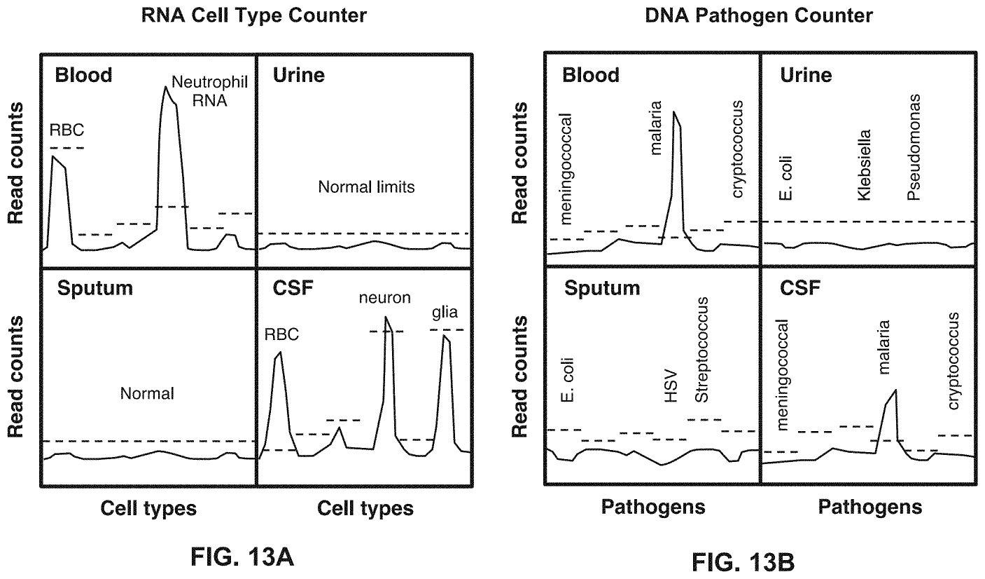

In some embodiments, real-time reporting of read counts can be used to display which cell types, responses and non-human pathogens have been detected thus far. These types of RNA and DNA data can, in some respects, parallel traditional laboratory-based tests where analogous inferences can be produced. Because different biological samples can provide different information, different panels can be used to represent different biological sample sites and display current total, tissue-specific and pathogen-specific read counts. For example, FIGS. 13A-13B illustrate an exemplary interface via which real-time visualization of RNA and DNA read counts can be output to a physician (the illustrated read counts are hypothetical and intended to be purely illustrative). The set of four panels in FIG. 13A illustrates exemplary outputs for an RNA cell type counter for four biological sample sites, e.g., respective read counts for cells in blood, urine, sputum, and cerebrospinal fluid (CSF). The set of four panels in FIG. 13B illustrates exemplary outputs for a DNA pathogen counter for four biological sample sites, which can be the same as or different than the biological sample sites for the RNA cell type counter, e.g., respective read counts for cells in blood, urine, sputum, and CSF. Within each inset, an exemplary, hypothetically expected cell type specific RNA or DNA read count in the presence of an infection, where both RBCs and a relatively large number of neutrophils are detected in blood, are illustrated. For example, in FIG. 13A, in blood, RNA read counts of multiple cell types including RBCs and neutrophils are displayed as peaks and change in height as more reads counts are detected. As another example, also in FIG. 13A, two panels, representative of urine and sputum, show no RNA read counts (low or flat peak). In a fourth panel of FIG. 13A, cerebrospinal fluid (CSF), visible peaks in the RNA cell type counter demonstrate the presence of RBC, neurons, and glia. Use of dashed lines demonstrate where detection thresholds are met and convey in the fourth panel that the amount of RBC RNA detected in the CSF is above a threshold of normal for CSF. This result would be suggestive of intracranial hemorrhage. The DNA pathogen counter, the output of which is illustrated in FIG. 13B, illustrates that relatively high levels of malaria are detected in blood and sputum, which the physician readily can use as part of making a diagnosis. In some embodiments, the use of these types of result reporting can follow along normal diagnostic paradigms currently used by physicians albeit using a non-traditional data in the form of RNA and DNA read counts.

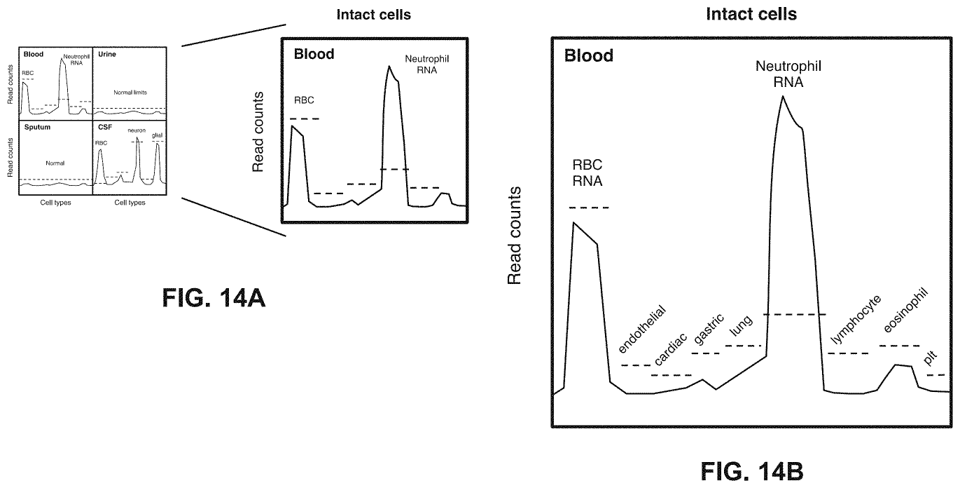

A similar metric used by laboratory assays is the proportion of cells within a biological sample. In the present devices and methods, an analogous metric can be exemplified by the differential complete blood count. In some embodiments, based on this assay, the percentages of several cell types found in blood can be reported to the physician. For example, FIGS. 14A-14B illustrate an interface in which interactive displays can allow the physician to select and magnify the categories examined. In some embodiments, while in an exemplary multi-panel view illustrated in FIGS. 13A-13B and in the FIG. 14A, only certain categories may be shown. Upon the physician's selecting or magnifying a single panel using an interface such as illustrated in FIG. 14B, additional categories can be displayed, or the selected results can be enlarged so as to provide increased detail of cell types tested. This information can facilitate the physician to understand what conditions have been screened and to use such data to exclude other possible diagnoses. The display of the presence of expected cells, such as red blood cells in blood, can also useful to the physician to confirm the biological source and to validate the assay. For example, FIG. 14B illustrates exemplary interactive results viewing and enlargement for increased detail of cell types tested, according to some embodiments. In the illustrated, nonlimiting example, by selecting the blood panel (upper left panel of the set of four panels illustrated at the far left of FIG. 14A or the upper left panel of FIG. 13A), the physician can be offered views to further his or her understanding of where read counts are derived from. For example, FIG. 14B illustrates an exemplary interface in which the read counts for additional different cell types for which RNA may be detected in the patient's blood (e.g., RBC, endothelial, cardiac, gastric, lung, neutrophil, lymphocyte, eosinophil, or platelet [plt]). In a first hypothetical example, RNA from RBCs comes predominantly from intact cells, which is a normal phenomenon. In other hypothetical scenarios, RBC RNA might be abnormally abundant. Such a result can occur, for example, in the setting of hemolytic types of diseases caused by autoimmune or adverse drug events or from poor sampling. In the case of poor sampling, other cell types such as neutrophils also can be affected and can serve as useful controls for sample quality.

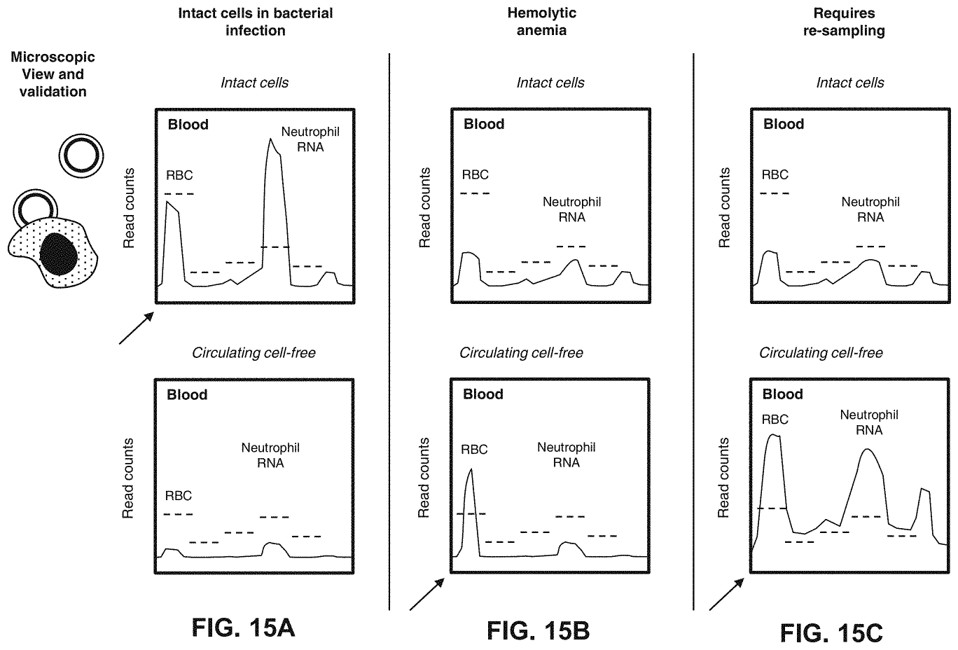

In some embodiments, an additional type of RNA and DNA data can be based on cell separating processes such as described further above with reference to FIGS. 5A-5B. For example, based on cell-separating processes, read counts from nucleic acids obtained from cell-intact vs. extracellular circulating compartments can be obtained. For example, FIGS. 15A-15C illustrate exemplary selected views of read counts from intact or circulating cell-free samples, according to some embodiments. The conceptual differences between intact and circulating cell-free samples may be familiar to physicians who are trained to recognize that low amounts of intact red blood cells are associated with a condition called anemia, and that presence of damaged cells or debris suggests a disease called hemolytic anemia. Physicians are also aware that post-sampling errors can cause artificial blood hemolysis. For example, the presence of other “lysed” cell types, e.g., neutrophil, can confirm a non-specific cause of cell lysis consistent with a post-sampling error.

FIGS. 15A-15C respectively illustrate an exemplary selected view of read counts and cells from intact (upper panels) or circulating cell-free (lower panels) samples, according to some embodiments. The upper panel of the exemplary interface illustrated in FIG. 15A illustrates hypothetical exemplary read counts of RNA from neutrophils and RBCs from intact cells of a hypothetical “normal” individual, while the lower panel illustrates exemplary read counts of circulating cell-free RNA from neutrophils and RBCs for that individual. It can be seen that the RNA counts from the RBCs and neutrophils are primarily predominantly from intact cells and are representative of cell number. An exemplary visual confirmation of this result obtained during sample preparation described in Component 2 can be displayed. The exemplary interface illustrated in the upper panel of FIG. 15B illustrates hypothetical exemplary read counts of RNA from neutrophils and RBCs from intact cells of a hypothetical individual with hemolytic anemia, while the lower panel illustrates exemplary read counts of circulating cell-free RNA from neutrophils and RBCs of that individual. It can be seen that the circulating cell-free RNA counts from the RBCs of that individual are abnormally high. Such a result can be interpreted as being suggestive of hemolysis such as can be seen in autoimmune disease, e.g., autoimmune hemolytic anemia (as in the present, nonlimiting example); adverse drug events, e.g., drug induced hemolysis; or from poor technical sampling. In this case, the results can be interpreted as indicating that poor sampling is unlikely, because other cell types such as neutrophils can be observed not to be affected (e.g., the read count of circulating cell-free RNA is similar to that of the normal individual). The exemplary interface illustrated in the upper panel of FIG. 15C illustrates hypothetical exemplary read counts of RNA from neutrophils and RBCs from intact cells of a hypothetical normal individual for which the blood sampling was poor, while the lower panel illustrates exemplary read counts of circulating cell-free RNA from neutrophils and RBCs of that sample of that individual. It can be seen that the circulating cell-free RNA counts from one or more cell types, e.g., from the RBCs and neutrophils of that individual, are abnormally high, while the intact cell RNA counts from the RBCs and neutrophils of that individual are abnormally low. As such, the poor sample integrity can be understood to result in cell damage to RBCs and neutrophils and can be identified by RNA detection in the circulating cell free compartment. For example, the user can detect poor sample integrity based on data obtained internally by machine, one exemplified by comparing the results of cell-free vs. cell-intact compartments.

Note that some embodiments include displaying reports or results such as illustrated in FIGS. 15A-15C, in which a threshold is shown for which abnormal values are found above the threshold and normal values found below. In some embodiments, such threshold values can reflect values expected for the current number of cells analyzed. In some embodiments, such threshold values can be pre-populated from laboratory-tested standards and from aggregated data from the use of device. In some embodiments, in addition to thresholds, the number of supporting samples or observations can displayed to the physician so as to serve as a reference. In some embodiments, supporting observations can incorporate previous diagnoses and previous validated diagnoses from the aggregate medical record.

Illustratively, a method for use in assessing the quality of a biological sample obtained from a subject, the biological sample including nucleic acids, the method being executed by a device (e.g., an instrument such as described herein with reference to FIGS. 6A-9B), can include quantifying by the device an amount of a first subset of the nucleic acids that are present in the biological sample, the first subset of the nucleic acids having an intracellular origin. The method also can include quantifying by the device an amount of a second subset of the nucleic acids that are present in the biological sample, the second subset of the nucleic acids having an extracellular origin. The method also can include outputting by the device an indication of the amount of the first subset of the nucleic acids; and outputting by the device an indication of the amount of the second subset of the nucleic acids. The relative amounts of the first and second subsets of the nucleic acids can indicate the quality of the biological sample. Optionally, the method also includes outputting by the device an indication of an expected amount of the first subset of the nucleic acids in a normal biological sample and an indication of an expected amount of the second subset of the nucleic acids in a normal biological sample.

Under another aspect, a device (e.g., an instrument such as described herein with reference to FIGS. 6A-9B) for use in assessing the quality of a biological sample obtained from a subject, the biological sample including nucleic acids, can include a first quantification module configured to quantify an amount of a first subset of the nucleic acids that are present in the biological sample, the first subset of the nucleic acids having an intracellular origin, e.g., components 5A and 5B. The device also can include a second quantification module configured to quantify an amount of a second subset of the nucleic acids that are present in the biological sample, the second subset of the nucleic acids having an extracellular origin, e.g., components 5A and 5B. The device also can include an output module configured to output an indication of the amount of the first subset of the nucleic acids and to output an indication of the amount of the second subset of the nucleic acids, e.g., display component 6 that displays the indications, a computer readable medium that stores the indications, or component 7 that transmits the indications to a computer. The relative amounts of the first and second subsets of the nucleic acids indicate the quality of the biological sample. Optionally, the output module further is configured to output an indication of an expected amount of the first subset of the nucleic acids in a normal biological sample and an indication of an expected amount of the second subset of the nucleic acids in a normal biological sample.

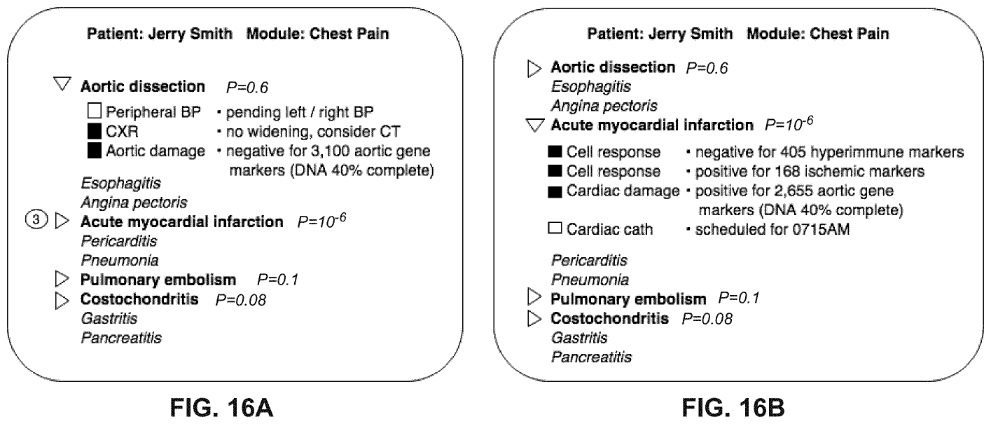

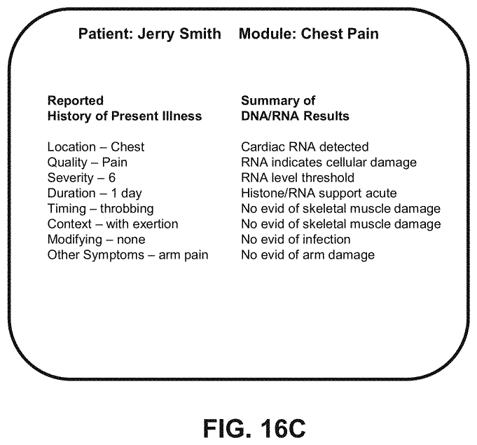

In some embodiments, upon activation of the device and the entry of chief symptoms and site via input from the physician, an automated search of patient electronic records can begin to identify, and optionally to self-complete, key clinical determinants. These determinants can include, but are not limited to, one or more of the following: history of immunocompromised states, recent infections, recent procedures, or other existing conditions. In some embodiments, any suitable combination of such elements can be processed so as to generate a list of possible diagnoses (with modifiers) and a matrix containing expected results from one or more types of biological samples and past specificity and sensitivity for each diagnosis. In some embodiments, for various possible diagnoses, the physician can select test values and results from the instrument that potentially may support or exclude each such diagnosis. The product of this interaction is a documented logic tree, which the physician creates as a result report, nonlimiting examples of which are illustrated in FIGS. 16A-16B. In one exemplary interaction with the device, the physician can be assisted in creating a results report using data generated by the device and concurrently though the medical record. Exemplary displays of this interaction are illustrated in FIGS. 16A-16B with active, possible diagnoses indicated in bold and inactive, excluded diagnoses in italics. In this example, numbers and triangles are used to identify diagnoses with new updated data and to expand current status. In example shown in FIG. 16A, under the diagnoses of aortic dissection, pending Peripheral BP, completed CXR from the medical record, and device assessment of aortic damage are displayed. DNA percent (%) completion communicates with the physician the completeness of the analysis, whereas exemplary displayed statistical values can be used to communicate probability of diagnoses. In the example shown in FIG. 16B, under ‘Acute myocardial infarction,’ hypothetical exemplary inferences drawn from RNA and DNA data are shown as well as pending tests or procedures (e.g., cardiac cath). The physician may also choose to add additional data from the device to support his or her diagnostic recommendation.

In the nonlimiting examples illustrated in FIGS. 16A-16B, the patient is suffering from myocardial infarction or heart attack. The provider inputs “chest pain” as a chief complaint and an automated search of patient electronic records by the device reveals no prior history of infection or immunocompromised state. A list of possible diagnoses, including myocardial infarction, pneumonia, pulmonary embolism, aortic dissection, cardiac tamponade, costochondritis, peptic ulcer, and a matrix of expected results for each scenario are communicated in real-time to the provider. The displayed expected results would display the normal number of cardiac, pulmonary, gastric, arterial vasculature, bacterial, viral, inflammatory and hematologic RNA transcripts expected in normal blood and the diagnostic level and pattern of RNA transcripts associated with tissue-specific damage for each diagnostic possibility. In other display modes, the number of patient samples already examined, specificity and sensitivity data can be displayed.

In various embodiments, the use of the result output also serves to provide several functions. One exemplary function is to allow the physician to use RNA and DNA results to infer the same types of diagnostic knowledge as traditional laboratory tests. This structured output of data along side with patient records are readily adopted into diagnostic algorithms already familiar to physicians and similarly trained health professionals. Another exemplary function is to allow the physician to highlight which views and results are most informative to the physician. In some embodiments, such a function can be generated through scoring choices and interactions used by the physician or alternatively, the physician can choose to store or flag views which document their diagnostic conclusions.

Another exemplary function displayed in FIG. 16C allows the physician to use RNA and DNA results to infer or to relate to the same diagnostic characteristics normally described by the patient's symptoms. Characteristics of symptoms are defined by several sources, including standard medical textbooks and established medical institutions such as the Center for Medicare and Medicaid Services (CMS). Such symptom characteristics as location (site of symptoms), quality (pain, itching, color, etc.), severity, duration, context, timing, modifiers, and accompanying symptoms can be correlated to RNA and DNA tests as described in FIGS. 4A-4C and 5A-5B. Like anatomical data, symptom characteristics and their range of acceptable values are well-established as evidenced by tools to aid in medical documentation by physicians and patients (REF). The display of RNA and DNA test results correlated with symptom characteristics allow the user to quickly identify and exclude sources for the patient's symptoms (FIG. 16C).

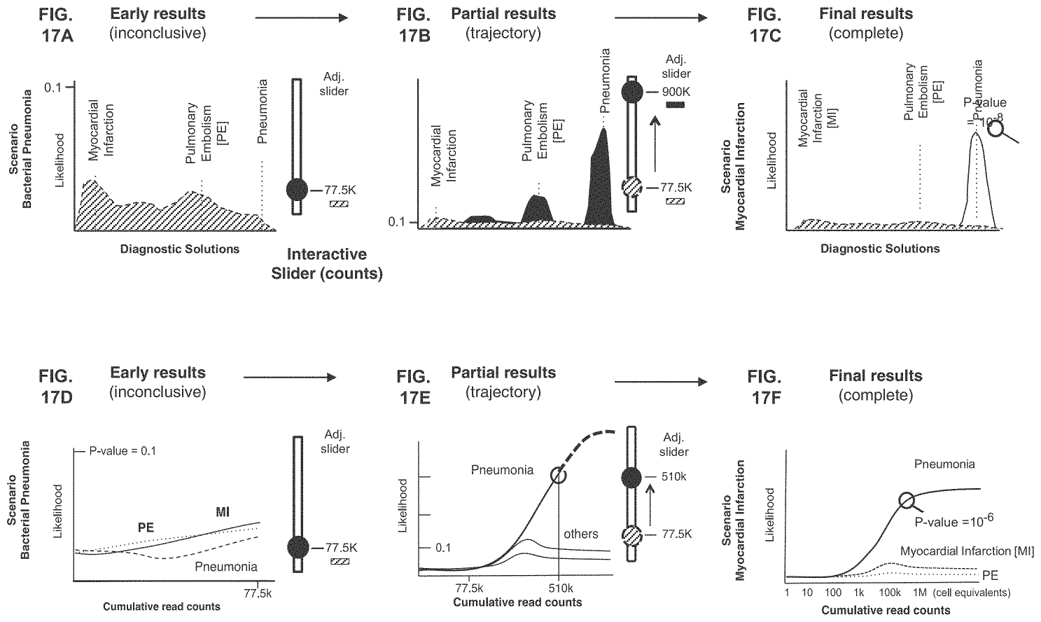

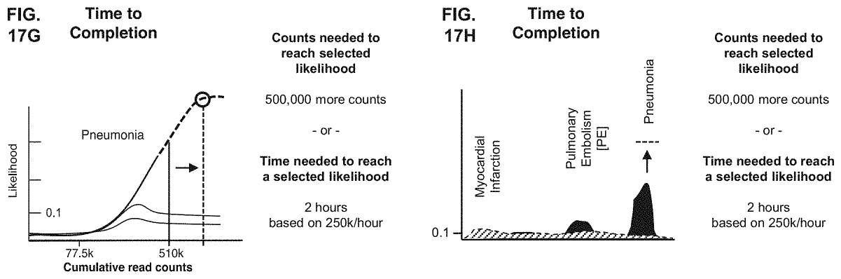

During the operation of the device, real-time display of RNA and DNA results are displayed for several clinical uses, including but not limited to determining the diagnosis, excluding diagnosis, viewing the status of the test, viewing the progress of the test, and creating reports. For example, FIGS. 17A-17F illustrate examples of intermediate and final stages of nucleic acid test result displays according to some embodiments. These examples illustrate how RNA and DNA results can be expressed to convey the progress of the test, the completeness of the test, and the significance of the current result. In FIG. 17A, an exemplary report displays a histogram of early RNA and DNA counts suggestive of a diagnosis. Examples of ‘counts’ include primary detection of molecules identified (e.g., FASTQ reads) or inferred detection of molecules (e.g., aligned reads). Several features describe the significance of this stage of results. The horizontal axis displays the categories of entities, exemplified by context-specific diagnostic possibilities. Other nonlimiting examples include cell types or microbial organisms. The vertical axis displays statistical significance as derived by one or more well-established methods including but not limited to: Fisher exact test, such as described in Fisher, “The logic of inductive inference,” J. R. Stat. Soc. 98: 39-82 (1935), the entire contents of which are incorporated by reference herein; False Discovery Rate, such as described in Benjamini et al., “Controlling the false discovery rate: a practical and powerful approach to multiple testing,” J. R. Statist. Soc. B, 57: 289-300 (1995), the entire contents of which are incorporated by reference herein. Over time, the number of nucleic acid ‘counts’ will increase as the device continues to detect more RNA or DNA and as more material (e.g., cells, sample volume, consumption of reagents, general or specific number of nucleotides or molecules detected) is analyzed.

To portray results in a manner easily understood by the user, the number of counts is displayed in relation to a direct or indirect number of cells analyzed. The number of counts per cell is pre-established based on laboratory observations from known inputs of titrated cell numbers. For example, 1000 counts might be equivalent to 5 human lab reference cells. As illustrated in FIG. 17A-17B, the increase in the number of nucleic acid counts over time from FIG. 17A to FIG. 17B is displayed as an increase from 77,500 (77.5k) cell equivalents analyzed to 900,000 (900k) cell equivalents analyzed. In relation to increasing amount of cell equivalents analyzed the likelihood may increase. In FIG. 17A, when results based on an equivalent of 77.5k cells are still inconclusive, a higher probability of ‘pneumonia’ is observed after an equivalent of 900k cells is reported in FIG. 17B. In FIG. 17C, when nucleic acid counts equivalent to 1 million (M) cells are analyzed, a statistical probability (denoted with a magnifying glass icon) of pneumonia is displayed as the most likely condition (P=10⁻⁸). An exemplary interface, such as a virtual slider, further illustrates the differences between the likelihood of results based on a smaller number of cell equivalents compared to a much larger number of cell equivalents. In some examples, selecting a quantity of cell equivalents to a higher number will have negligible changes in the probability of a diagnostic conclusion as might occur when the probability is near maximal. In other cases, movement of the slider and changing the number of cell equivalents will have significant changes in probability of a result.

In another example of representing real-time nucleic acid results (FIG. 17D-17F), the progression of results is shown as increasing number of counts (in cell equivalents or other measure). The diagnostic likelihood can be shown in relationship with the number of the current measure of nucleic acid counts. As depicted in FIG. 17D, “early results” of possible diagnoses at 77,500 (77.5K) read counts were still inconclusive, whereas at 510,000 (510K) read counts, the partially complete results suggest a high likelihood of pneumonia and a trajectory that if continued sequencing is performed, the likelihood will continue to improve. Conversely, if the slope of this trajectory has plateaued such as shown in FIG. 17E, then the physician would see that continued operation of the device would not improve or change the likelihood of the diagnosis. FIG. 17E shows partial results when the 510,000 cell equivalents are examined not yet at the 1M cell equivalents (FIG. 17F) needed for maximal diagnostic likelihood. The trajectory of probability with additional nucleic acid counts however convey to the user that alternative possibilities are unlikely, allowing the user to act earlier with preliminary results.

An exemplary useful feature of displaying a trajectory of diagnostic likelihood based on a progression of nucleic acid results is that the remaining time necessary for diagnosis can be estimated. Given the rate of increasing likelihood vs. the number of nucleic acid counts, the amount of time can be calculated to reach a threshold of diagnostic completion, for example as illustrated in FIGS. 17G-17H. Based on mathematical model of plot of likelihood, an approximate rate of likelihood change can be calculated. For example, in a linear model, the rate of likelihood change or the slope can be used to derive the amount of likelihood change over the amount of ‘counts’. Similarly, in a polynomial model, the derivative at various points in the model can be used to determine when added ‘counts’ can improve the likelihood significantly or when added ‘counts’ can have little effect on likelihood. As additional medical data is included, e.g., either via entry of new information from the user or external data from the real-time medical record, the overall likelihood of each diagnoses can change. As the overall likelihood changes due to additional medical information, the impact of additional nucleic acid ‘counts’ can be re-assessed and the time needed to reach a threshold for a likelihood will be updated.

In some embodiments, the device can present a template for the physician to view or document which diagnoses are the most likely and which diagnoses are highly unlikely based on what the current status of findings. In the nonlimiting example of chest pain, the instrument can present a list of diagnoses, of which the physician can mark or view their respective likelihood, inability to exclude, and the like. Illustratively, each diagnostic choice can trigger a set of questions, such as “was there evidence of cardiac damage?”, “sign of infection?”, and others. In response to the questions, the physician can draw upon “flagged” views to support his or her report created from outputs shown in FIGS. 17A-17F.

One non-limiting example of “flags” on reporting views is that the physician can create reports which to document and support their diagnostic claims. For example, FIGS. 17A-17F illustrate examples of how nucleic acid test data can be included in a results summary (or report), according to some embodiments. FIGS. 17A-17F illustrate examples of how nucleic acid test data can be included in a results summary, according to some embodiments. FIG. 17A illustrates an exemplary likelihood-diagnosis histogram that can be used to display real-time data and mirrors the “early results” and “partial results” respectively shown in FIGS. 17D and 17E. In FIG. 17A, with only 77.5K read counts, the diagnosis is unclear. In FIG. 17B, an adjustable slider can show the trajectory of histogram from one time point (e.g., 77.5K) to another time point (e.g., 900K). In another exemplary display shown in FIG. 17C, a likelihood vs. diagnostic solutions histogram is shown with similar diagnoses grouped. Such a display allows the physician to identify the current status of the device and what diagnoses are being evaluated. For example, the peak labeled as “Myocardial Infarction [MI]” may represent several related diagnoses such as anterior MI, posterior MI, unstable angina, and others or alternatively, independent read count signatures which cumulatively point to MI as the likely diagnosis, or to pneumonia as the likely diagnoses in the illustrated example. As depicted in FIG. 17D, “early results” of possible diagnoses at 77,500 (77.5K) read counts were still inconclusive, whereas at 510,000 (510K) read counts, the partially complete results suggest a high likelihood of pneumonia and a trajectory that if continued sequencing is performed, the likelihood will continue to improve. Conversely, if the slope of this trajectory has plateaued such as shown in FIG. 17E, then the physician would see that continued operation of the device would not improve or change the likelihood of the diagnosis. In FIG. 17F, the physician generates a visual report to support their diagnosis of myocardial infarction. In this example, the physician cites RNA or gene read counts data denoted by i) an arrow, ii) a window of 1M cell equivalents, iii) an icon resembling a magnifying glass to cite the P-value associated with their reference, and other diagnoses considered. In FIG. 17D, in an exemplary pneumonia report, the physician chooses to show RNA read counts from blood as supporting evidence for pneumonia. The report also displays other conditions screened and a slider or range window to demonstrate at what stage (and time) was the diagnosis ambiguous and at what point did the diagnosis become well supported.

In some embodiments, some aspects of the diagnostic report can draw upon data or request additional data that is not generated by the device. For example, the current medical record can be automatically included as supporting or excluding evidence in the physician report. The physician can use a RNA or DNA result from the device as an alternative to a named laboratory test and vice versa. Thus, in certain embodiments, the generated report from the device incorporates both observations from the medical record and the device itself. In other embodiments, the device may accept and incorporate visual or electronic results such an exemplary chemical strip test or other complementary assays. These interactions can further support the use of specific RNA and DNA data types in replacement or in parallel with currently used laboratory tests.

Illustratively, a method for use in diagnosing a condition based on a symptom experienced by a subject and based on a biological sample obtained from the subject, the biological sample including nucleic acids, can be executed by a device (such as the instruments described herein with reference to FIGS. 6A-9B). The method can include, over a first period of time, quantifying by the device an amount of a first subset of the nucleic acids that are present in the biological sample, the first subset of the nucleic acids having a first origin. The method further can include, over the first period of time, quantifying by the device an amount of a second subset of the nucleic acids that are present in the biological sample, the second subset of the nucleic acids having a second origin that is different than the first origin. The method further can include outputting by the device an indication of the amount of the first subset of the nucleic acids quantified over the first period of time; and outputting by the device an indication of the amount of the second subset of the nucleic acids quantified over the first period of time.

In some embodiments, the method optionally, can include, based on the amount of the first subset of the nucleic acids quantified over the first period of time, estimating by the device a first likelihood that the subject is suffering from a first condition. The method optionally can include, based on the amount of the second subset of the nucleic acids quantified over the second period of time, estimating by the device a second likelihood that the subject is suffering from a second condition that is different than the first condition. The method optionally can include outputting by the device an indication of the first likelihood and an indication of the second likelihood.

Additionally, or alternatively, the method optionally can include, based on the amount of the first subset of the nucleic acids quantified over the first period of time, estimating by the device a first trajectory of an amount of the first subset of the nucleic acids over a second period of time. The method optionally can include, based on the amount of the second subset of the nucleic acids quantified over the first period of time, estimating by the device a second trajectory of an amount of the second subset of the nucleic acids over the second period of time. The method optionally can include outputting by the device an indication of the first trajectory and an indication of the second trajectory. Optionally, the method can include, based on the first and second trajectories, estimating by the device a second time at which the first or second condition is sufficiently likely as to make a diagnosis that the patient is suffering from that condition; and outputting by the device an indication of the second time.

The method also, or alternatively, can include receiving by the device additional clinical information regarding the patient, wherein the first and second likelihoods further are based on the received additional clinical information.

Additionally, or alternatively, the method optionally can include, over a second period of time subsequent to the first period of time, quantifying by the device an amount of the first subset of the nucleic acids that are present in the biological sample. The method optionally can include, over the second period of time, quantifying by the device an amount of the second subset of the nucleic acids that are present in the biological sample. The method optionally can include outputting by the device an indication of the amount of the first subset of the nucleic acids quantified over the second period of time; and outputting by the device an indication of the amount of the second subset of the nucleic acids quantified over the second period of time.

The indications of the amounts of the first and second subsets of nucleic acids quantified over the first period of time optionally can include a histogram, e.g., such as described herein with reference to FIGS. 17A-17C.

In some embodiments, the indication of the amount of the first subset of the nucleic acids over the first period of time includes a number of first cell equivalents, and the indication of the amount of the second subset of the nucleic acids over the first time includes a number of second cell equivalents. Optionally, the first origin can include a pathogen, and the number of first cell equivalents can represent a severity of infection of the subject by the pathogen. Additionally, or alternatively, the number of first cell equivalents or the number of second cell equivalents represents a severity of a condition from which the subject is suffering or clinical significance. Additionally, or alternatively, the number of first cell equivalents or the number of second cell equivalents represents a response to a treatment.

Under another aspect, a device (e.g., an instrument such as described herein with reference to FIGS. 6A-9B) for use in diagnosing a condition based on a symptom experienced by a subject and based on a biological sample obtained from the subject, the biological sample including nucleic acids, includes a first quantification module configured to quantify, over a first period of time, an amount of a first subset of the nucleic acids that are present in the biological sample, the first subset of the nucleic acids having a first origin, e.g., can include components 5A and 5B. The device also can include a second quantification module configured to quantify, over the first period of time, an amount of a second subset of the nucleic acids that are present in the biological sample, the second subset of the nucleic acids having a second origin that is different than the first origin, e.g., can include components 5A and 5B. The device also can include an output module configured to: output an indication of the amount of the first subset of the nucleic acids quantified over the first period of time, and to output an indication of the amount of the second subset of the nucleic acids quantified over the first period of time, e.g., can include component 6 configured to display such an output, can include a computer-readable medium configured to store such an output, or can include component 7 configured to transmit such an output to a computer.

Optionally, the device also can include an estimation module configured to estimate, based on the amount of the first subset of the nucleic acids quantified over the first period of time, a first likelihood that the subject is suffering from a first condition, e.g., can include components 5A and 5B. The estimation module further can be configured to estimate, based on the amount of the second subset of the nucleic acids quantified over the second period of time, a second likelihood that the subject is suffering from a second condition that is different than the first condition. The output module further can be configured to output an indication of the first likelihood and an indication of the second likelihood. Additionally, or alternatively, the estimation module optionally further can be configured to estimate, based on the amount of the first subset of the nucleic acids quantified over the first period of time, a first trajectory of an amount of the first subset of the nucleic acids over a second period of time. The estimation module optionally further can be configured to estimate, based on the amount of the second subset of the nucleic acids quantified over the first period of time, a second trajectory of an amount of the second subset of the nucleic acids over the second period of time. The output module further optionally can be configured to output an indication of the first trajectory and an indication of the second trajectory. Optionally, the estimation module further can be configured to estimate, based on the first and second trajectories, a second time at which the first or second condition is sufficiently likely as to make a diagnosis that the patient is suffering from that condition; and the output module further can be configured to output an indication of the second time.

Additionally, or alternatively, the device further can include an input interface configured to receive additional clinical information regarding the patient, wherein the first and second likelihoods further are based on the received additional clinical information.

Additionally, or alternatively, the first quantification module optionally can be configured to quantify, over a second period of time subsequent to the first period of time, an amount of the first subset of the nucleic acids that are present in the biological sample. The second quantification module optionally can be configured to quantify, over the second period of time, an amount of a second subset of the nucleic acids that are present in the biological sample. The output module optionally can be configured to output an indication of the amount of the first subset of the nucleic acids quantified over the second period of time; and can be configured to output an indication of the amount of the second subset of the nucleic acids quantified over the second period of time.

Additionally, or alternatively, the indications of the amounts of the first and second subsets of nucleic acids quantified over the first period of time optionally include a histogram, e.g., such as described herein with reference to FIGS. 17A-17C.

Optionally, in some embodiments, the indication of the amount of the first subset of the nucleic acids over the first period of time includes a number of first cell equivalents, and the indication of the amount of the second subset of the nucleic acids over the first time includes a number of second cell equivalents. Additionally, or alternatively, the first origin can include a pathogen, and the number of first cell equivalents can represent a severity of infection of the subject by the pathogen. Additionally, or alternatively, the number of first cell equivalents or the number of second cell equivalents represents a severity of a condition from which the subject is suffering or clinical significance. Additionally, or alternatively, the number of first cell equivalents or the number of second cell equivalents represents a response to a treatment.

An exemplary parameter in evaluating the significance of a result is understanding the evidence supporting a particular diagnostic solution. For example, the evidence can include recommendations from an established committee and publications, based on large controlled studies. These types of supporting evidence can be accessible through the device interface, e.g., via network module (Component 7). Another type of evidence that can grow over time is the increasing numbers of samples and results obtained by the aggregate of users of the device. This type of data can be displayed to the physician and incorporated with the generated report, and can include, for example, aggregate results such as the frequency of specific findings in other cases with confirmed diagnosis, e.g., discharge continuity of care or equivalent documents, improvement of condition in response to treatment, and the like.

In certain aspects, the generated report from the device can further comprise a risk score based on the expression information. In particular aspects, the risk score may be defined as a weighted sum of expression levels of biomarkers. For example, the risk score may be calculated based on a summation of the expression level of the selected biomarkers multiplied with a corresponding regression coefficient. The regression coefficient may be calculated according to a regression analysis of the correlation between the expression level of the biomarker genes and survival of a control group. To improve data processing efficiency, the risk score can be generated on a computer.

Longitudinal studies can be performed and yield consecutive reports as biomarkers can be repeatedly taken from patients at multiple points in time. In longitudinal studies, a small set of biomarkers is correlated to the disease progression and that biomarkers expressed at different stages can be of prognostic value with regard to therapy resistance.

Post-Diagnostic, Self-Learning of a Symptom-Based Diagnostic Device

Data from the device is another source of new knowledge. Also, as noted in earlier sections, data from the continued use of the device on multiple patients and by multiple physicians can be informative. For example, new data from users can be aggregated to quantify their concordance or discordance with specific interpretations of RNA and DNA data. Illustratively, these interpretations can occur at the inference level of which tissue was affected, what host response was present or what pathogens were present or at the diagnostic level. In some embodiments, at the inference value level, sequences that are identified as poorly concordant can be discarded from future devices or edited to improve sensitivity or specificity. In some embodiments, at the diagnostic level, the interpretation of discordant sequences can be altered to reflect supporting data. In both cases, the level of concordance can be reported with results to aid the physician in understanding the strength or weakness of each data point.

Some pre-computed sequences can be difficult to detect in practice for technical reasons. Other scenarios, e.g., different locations, hospital vs. clinic or in the United States vs. another country, potentially can have different prevalence or unique exposure to diseases, which are not initially pre-computed. For example, some types of infections can differ in frequency or in pathogen in different areas of the world. In a common type of wound, e.g., genital ulcers, the causes can differ in likelihood based upon factors such as geographic location, and diagnoses can range, e.g., from herpes simplex virus to syphilis or other pathogens.

Thus, in some embodiments, the geographic and context for each result can be taken into account, such as via global positioning systems, internet protocol address (IP), or other identifiers of location. Storage of data with biological sample site, geolocation, symptoms, and medical criteria metadata can facilitate self-learning.

In some embodiments, upon completion of diagnostic run of device, the device can be returned for re-charging and re-use. During this process, one or more types of data can be collected for post-diagnosis improvements, including, but not limited to, one or more of: electronic medical records from discharge diagnoses, physician interaction or contributed data, or raw biological material remaining in the device.

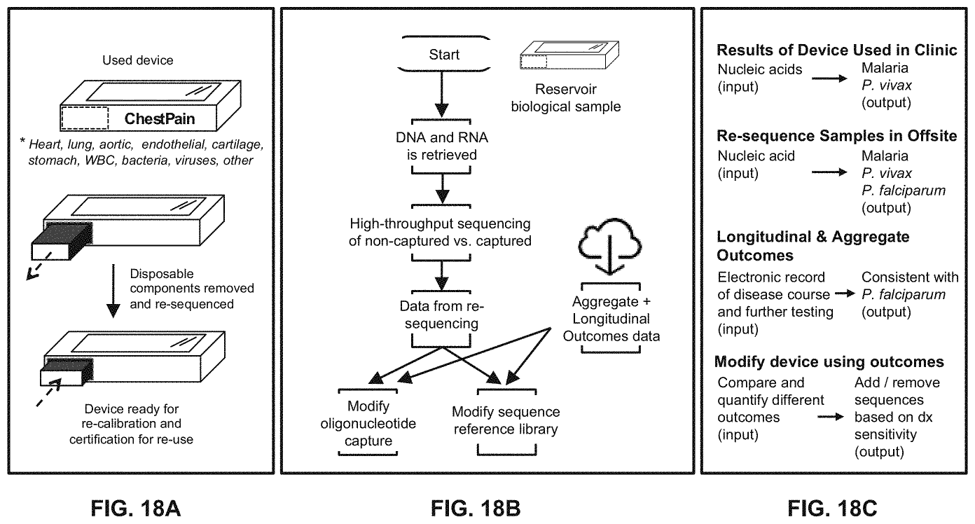

As another example, the remaining biological material can be used for further analysis. For example, non-targeted and targeted sequencing can be performed to identify new sequences, which can be more diagnostic, as described in greater detail below with reference to FIGS. 18A-18C. This latter step can used to identify nucleic acids that were not captured for targeted sequencing. In some embodiments, a more comprehensive sequencing approach can be used to identify new potential clinically relevant sequences. Illustratively, analysis of targeted vs. non-selective sequences can drive improved targeted sequencing and nucleic acid detection. In some embodiments, remaining captured and uncaptured nucleic acids will be processed by sequencing or other nucleic acid analyzer to score sensitivity and specificity of context-specific devices.

So as to improve performance and accuracy of nucleic acid based testing, an output for modifications can be used. For example, FIGS. 18A-18C illustrate an exemplary self-learning process to improve or optimize capture, identification, and interpretation using outcomes data and re-sequencing, according to some embodiments. For example, in some embodiments, used devices such as illustrated in FIG. 18A, can include components from such devices that can be designed to be readily exchanged and restored for use. These components would contain useful genetic material for optimizing future sequence performance, analysis, and diagnostic assistance. For example, the residual or archived biological samples can provide a reservoir of useful genetic material that can be used for future optimization of sequence performance, analysis and diagnostic assistance. As illustrated in FIG. 18B, in some embodiments, biological samples can be re-sequenced using external sequencing instrumentation to obtain a fuller spectrum of captured versus non-captured nucleic acids. Illustratively, longitudinal data, e.g. patient discharge records, and aggregate data from other patients can be used to improve sensitivity and specificity of the device. In some embodiments, improvements can be implemented by modifications in nucleotide targeted sequencing or capture, and by modifying datasets to recognize more specific or highly sensitive sequences. Improvements can be implemented by modifications in nucleotide targeted sequencing or capture and by modifying datasets to recognize more specific or highly sensitive sequences. FIG. 18C illustrates an example of a sequence of events comparing the output of the device output, re-sequenced samples, and longitudinal and aggregate data to the recognition of sequences with high or low diagnostic value.

Based on the evaluation of device accuracy and sensitivity, a self-learning model such as illustrated in FIGS. 18B-18C potentially can facilitate modifications at several steps. For example, if new sequences with high clinical value are identified, new reagents for targeted sequencing can be provided. Additionally, confounding sequences potentially can be eliminated. Additionally, modifications to oligonucleotides for capture or amplification potentially can be made so as to re-activate the device with new reagents. Other modifications can be made at the level of which sequences are used in diagnostic interpretation, e.g., changes in symptom-specific sequence recognition, or alter the interpretation of specific sequences, e.g. change in where tissue damage is inferred. Additional refinements are made in the reporting and diagnostic interfaces with the physician to improve content and provide context- or geo-specific diagnostic support.

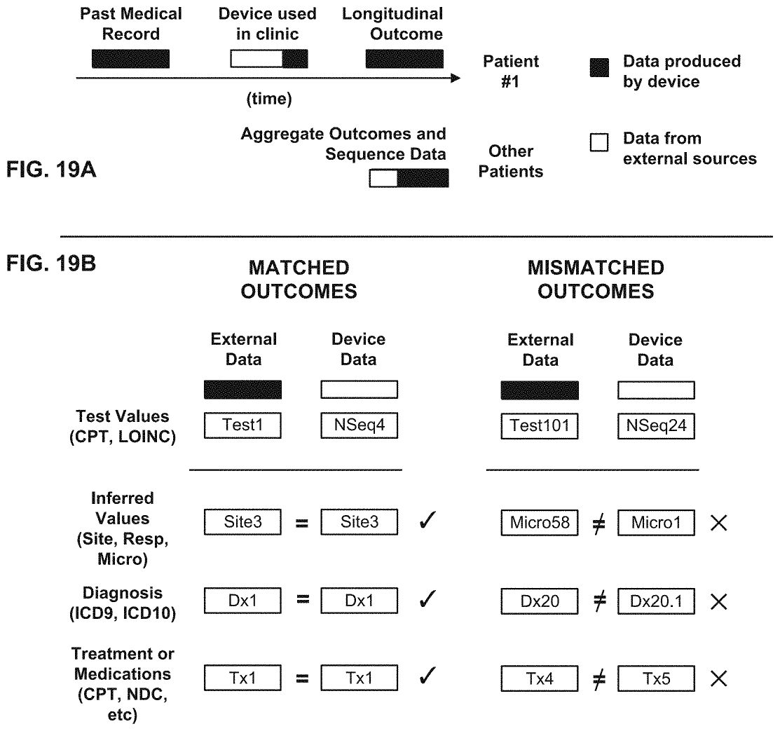

FIGS. 19A-19B illustrate an exemplary comparison of longitudinal and aggregate electronic outcomes data to RNA-DNA values, according to some embodiments. Illustratively in FIGS. 19A-19B, longitudinal and aggregate data can be obtained from discharge information (discharge diagnoses) which include one or more electronic data fields suitable for the collection of confirmatory or novel test or procedural data, e.g., International Statistical Classification of Diseases Version 9 and 10 (ICD9, ICD10), Logical Observation Identifiers Names and Codes (LOINC) and Current Procedural Terminology (CPT) field data. This medical record data can be used to cross-validate results from nucleic acid data. Another type of medical record data can be symptomatic information such as location, quality, severity, timing, duration, context, and others which are required in a portion of the patient record called the ‘history of present illness’ or HPI. This information is often input by the provider although in some settings, the patient can directly report symptoms (REF). RNA and DNA test values and quantity can be correlated with symptomatic characteristics as described in FIGS. 16A-16C. Another exemplary type of data, physician generated reports or feedback, e.g., final report summary inputted on device or on registered site, can be further incorporated into identifying which tests were highly informative and which can benefit from further optimization. For example, physician-based data can also be used to cross-identify which specific tests (e.g., LOINC, CPTs) were in agreement or incongruous with RNA/DNA-test values based on whether they share the same inferred value. For example, inferred values, site, host response, and pathogen, if shared by LOINC/CPT and RNA/DNA-based sets, potentially can provide a translational bridge between two types of data, traditional laboratory tests and nucleic acid-based tests.

For example, FIGS. 19A-19B illustrate an exemplary relationship between electronic record and RNA-DNA values, according to some embodiments. FIG. 19A illustrates longitudinal and aggregate (external) data including claims or electronic medical records related to patient and patients are compared to results produced from the device, e.g., a timeline of data obtained from the patient's past and later stages of care. This data is compared to data obtained from the patient using the device. FIG. 19B illustrates examples of comparison of outcomes between external sources and data produced from device, e.g., comparisons between CPT, LOINC data, symptomatic characteristics, and RNA-DNA values to identify which types of data can be used to infer the same clinical knowledge and medical diagnoses (ICD9, ICD10), using a newly defined inferred data category and value. In addition, mismatches. Inferred values generated from CPT, LOINC, ICD9, ICD10, medications, symptomatic characteristics, and RNA-DNA values are tested for matched or mismatched outcomes. In the examples, different tests, inferred values, diagnoses, and treatments are uniquely numbered. Nseq values represent a set of diagnostic sequences, e.g., Nseq4. In the first comparison, inferred values, diagnosis, and treatments match between external and device generated outcomes as indicated by checkmark. The result of this comparison is to add this sample to an aggregate counter for number of matches between device and external data. In the second comparison, there are mismatches between the inferred values, diagnoses and treatments as indicated by “incorrect” checkmark. The result can be recorded for example as a mismatch or decreased matching score between the NSeq24 set of sequences.

Illustratively, a method for performing one or more nucleic acid tests based on one or more symptoms experienced by a patient includes receiving by a device (e.g., an instrument such as described herein with reference to FIGS. 6A-9B) respective identifiers of the one or more symptoms experienced by the patient. The method also can include, by the device, submitting to a database a query based on the respective identifiers of each of the one or more symptoms, the database comprising a computer-readable medium storing at least a plurality of symptoms, a nucleic acid sequence associated with each of the symptoms, a potential diagnosis associated with each of the symptoms, a laboratory test or a procedure for each of the symptoms, and inferred data for each of the symptoms, the inferred value comprising a clinical inference based on a result of said laboratory test for the respective symptom. The method also can include, by the device, receiving from the database a response to the query, the response comprising one or more nucleic acid tests based on the nucleic acid sequences respectively associated with the one or more symptoms identified in the query. The method also can include, by the device, outputting respective representations of the one or more nucleic acid tests. The method also can include receiving, by a receptacle of the device, a cartridge configured to perform at least one of the one or more nucleic acid tests.

Optionally, the method further can include performing by the device the at least one of the one or more nucleic acid tests. The performing can include quantifying by the device an amount of a first subset of the nucleic acids that are present in the biological sample, the first subset of the nucleic acids having a first origin. The performing also can include quantifying by the device an amount of a second subset of the nucleic acids that are present in the biological sample, the second subset of the nucleic acids having a second origin. The method also can include determining by the device at least one possible diagnosis based on the amount of the first subset of the nucleic acids and based on the amount of the second subset of the nucleic acids. The method also can include outputting by the device an indication of the at least one possible diagnosis. The method also can include, by the device, receiving an indication of at least one of: a diagnosis made by the caregiver, a result of a laboratory test or a procedure performed on the subject, a symptomatic code, a site of injury, a cellular response, a host-immune response, a contribution of a non-human organism, or an origin of cells or symptoms. The method also can include transmitting by the device to the database the received indication for use in updating the database.

Optionally, the method further can include receiving by the device or by a second device respective identifiers of one or more symptoms experienced by a second patient, wherein the symptoms experienced by the second patient are the same as the symptoms experienced by the first patient. For example, although the database was updated using information provided by the previously mentioned device, the updated database subsequently can be accessed by the same device, or by a different device, in association with performing nucleic acid tests based on symptoms and a biological sample from another patient. The method further can include by the device or by the second device, submitting to the updated database a second query based on the respective identifiers of each of the one or more symptoms. The method further can include, by the device or by the second device, receiving from the updated database a response to the second query, the response comprising one or more updated nucleic acid tests based on the nucleic acid sequences respectively associated with the one or more symptoms identified in the second query, wherein at least one of the one or more updated nucleic acid tests is different than at least one of the one or more nucleic acid tests. The method also can include, by the device or by the second device, outputting respective representations of the updated one or more nucleic acid tests. The method also can include receiving, by the receptacle of the device or by a receptacle of the second device, a second cartridge configured to perform at least one of the updated one or more nucleic acid tests.

Under another aspect, a device (e.g., an instrument such as described herein with reference to FIGS. 6A-9B) for performing one or more nucleic acid tests based on one or more symptoms experienced by a patient can include an input module configured to receive respective identifiers of the one or more symptoms experienced by the patient, e.g., input component 6. The device also can include a query module configured to submit to a database a query comprising the respective identifiers of each of the one or more symptoms. The database can include a computer-readable medium storing at least a plurality of symptoms, a nucleic acid sequence associated with each of the symptoms, a potential diagnosis associated with each of the symptoms, a laboratory test or a procedure for each of the symptoms, and inferred data for each of the symptoms, the inferred value comprising a clinical inference based on a result of said laboratory test for the respective symptom. For example, component 5A-5B of the device can include such a query module that is configured to access the database (which optionally can be remote) via component 7. The query module further can be configured to receive from the database a response to the query, the response comprising one or more nucleic acid tests based on the nucleic acid sequences respectively associated with the one or more symptoms identified in the query. The device further can include an output module configured to output respective representations of the one or more nucleic acid tests. For example, the device can include display component 6 configured to display such output to a caregiver, or can include a computer-readable medium to which the output may be recorded, or can include a communication module, e.g., component 7, via which the device can provide the output to another computer or another computer-readable medium. The device further can include receptacle configured to receive a cartridge configured to perform at least one of the one or more nucleic acid tests, e.g., a receptacle for receiving one or more symptom-specific modules 9.

Optionally, the cartridge can include a first nucleic acid capture module configured to capture a first subset of the nucleic acids that are present in the biological sample (e.g., component 3), the first subset of the nucleic acids having a first origin. The cartridge further can include a second nucleic acid capture module configured to capture a second subset of the nucleic acids that are present in the biological sample (e.g., component 3), the second subset of the nucleic acids having a second origin. The device further can include a nucleic acid quantifier configured to quantify a respective amount of each of the first and second subsets of captured nucleic acids (e.g., components 5A-5B). The device further can include a diagnosis module (e.g., components 5A-5B) configured to determine at least one possible diagnosis based on the amount of the first subset of the nucleic acids and based on the amount of the second subset of the nucleic acids. The output module can be configured to output an indication of the at least one possible diagnosis. The input module further can be configured to receive an indication of at least one of: a diagnosis, a result of a laboratory test or a procedure performed on the subject, a symptomatic code, a site of injury, a cellular response, a host-immune response, a contribution of a non-human organism, or an origin of cells or symptoms. The query module further can be configured to transmit by the device to the database the received indication for use in updating the database.

Optionally, the input module further can be configured to receive respective identifiers of one or more symptoms experienced by a second patient, wherein the symptoms experienced by the second patient are the same as the symptoms experienced by the first patient. The query module further can be configured to submit to the updated database a second query based on the respective identifiers of each of the one or more symptoms. The query module further can be configured to receive from the updated database a response to the second query, the response comprising one or more updated nucleic acid tests based on the nucleic acid sequences respectively associated with the one or more symptoms identified in the second query, wherein at least one of the one or more updated nucleic acid tests is different than at least one of the one or more nucleic acid tests. The output module further can be configured to output respective representations of the updated one or more nucleic acid tests. The receptacle of the device further can be configured to receive a second cartridge configured to perform at least one of the updated one or more nucleic acid tests, e.g., a second component 9.

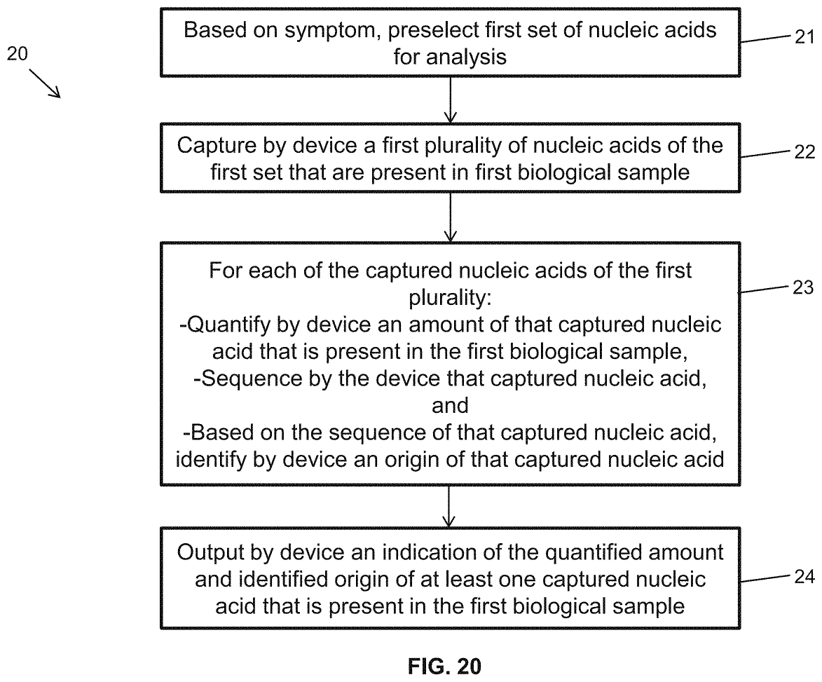

FIG. 20 illustrates an exemplary method for use in diagnosing a condition based on a symptom experienced by a subject and based on a first biological sample obtained from the subject, according to some embodiments. Method 20 illustrated in FIG. 20 can be executed by a device, e.g., such as described above with reference to any of FIGS. 6A-6C, 7, 8, 9A-9B, 10A-10D, 11A-11C, 12A-12B, 13A-13B, 14A-14B, 15A-15C, 16A-16C, 17A-17H, 18A-18C, and 19A-19B. Method 20 can include based on the symptom, preselecting a first set of nucleic acids for analysis (step 21). For example, in some embodiments, the device can include a first set of complementary nucleic acids configured to capture the first set of nucleic acids, the first set of nucleic acids being based on the symptom. Illustratively, the first set of complementary nucleic acids can be bound suitably to a portion of the device configured to receive the first biological sample or a portion thereof.

Method 20 further can include capturing by the device a first plurality of nucleic acids of the first set that are present in the first biological sample (step 22). For example, in some embodiments, the first set of complementary nucleic acids can capture a first plurality of nucleic acids of the first set that are present in the first biological sample.

Method 20 further can include, for each of the captured nucleic acids of the first plurality, quantifying by the device an amount of the captured nucleic acid that is present in the first biological sample; sequencing that captured nucleic acid; and, based on the sequence of that captured nucleic acid, identifying by the device an origin of that captured nucleic acid (step 23). For example, in some embodiments, the device can include a nucleic acid quantifier configured to quantify an amount of each of the captured nucleic acids that is present in the first biological sample. In some embodiments, the device also can include a nucleic acid sequencer that is configured to sequence each captured nucleic acid that is present in the first biological sample. In some embodiments, the device also can include a processor coupled to the quantifier and to the sequencer and being suitably programmed to identify an origin of each captured nucleic acid based on the sequence of that captured nucleic acid.

Method 20 further can include outputting by the device an indication of the quantified amount and the identified origin of at least one captured nucleic acid that is present in the first biological sample (step 24). For example, the device further can include a display coupled to the processor, the processor further being suitably programmed to cause the display to output an indication of the quantified amount and the identified origin of at least one captured nucleic acid that is present in the first biological sample.

In some embodiments, method 20 optionally can include preselecting the first set of nucleic acids for analysis comprises receiving by the device a first symptom-specific cartridge comprising a first set of complementary nucleic acids configured to capture the first set of nucleic acids for analysis. Optionally, method 20 further comprises, after the outputting step, removing the first symptom-specific cartridge from the device and receiving by the device a second symptom-specific cartridge comprising a second set of complementary nucleic acids. For example, in some embodiments, the device is configured to receive the first set of complementary nucleic acids within a first symptom-specific cartridge. Optionally, the first symptom-specific cartridge is removable and replaceable with a second symptom-specific cartridge comprising a second set of complementary nucleic acids. Optionally, the first set of complementary nucleic acids is different than the second set of complementary nucleic acids.

In some embodiments, method 20 optionally includes outputting by the device an indication of the quantified amount of each of the captured nucleic acids of the first plurality. For example, in some embodiments, the processor further is suitably programmed to cause the display to output an indication of the quantified amount of each of the captured nucleic acids of the first plurality.

In some embodiments, the capturing step (step 22) of method 20 comprises separating extracellular nucleic acids in the first biological sample from intracellular nucleic acids in the first biological sample; and the quantifying and sequencing steps are performed separately on the separated extracellular nucleic acids and on the intracellular nucleic acids. Optionally, the method further includes outputting by the device an indication of the quantified amount of at least one of the extracellular nucleic acids and an indication of the quantified amount of at least one of the intracellular nucleic acids. For example, in some embodiments, the device further includes a separator configured to separate extracellular nucleic acids in the first biological sample from intracellular nucleic acids in the first biological sample. Optionally, the nucleic acid quantifier and nucleic acid sequencer separately operate on the separated extracellular nucleic acids and on the intracellular nucleic acids. Optionally, the processor further is suitably programmed to cause the display to output an indication of the quantified amount of at least one of the extracellular nucleic acids and an indication of the quantified amount of at least one of the intracellular nucleic acids.

In some embodiments, the identifying by the device the origin of the captured nucleic acid (step 23 of method 20) comprises comparing the sequence of that nucleic acid to sequences stored in a library stored in a computer-readable medium of the device. For example, in some embodiments, the device further includes a computer-readable medium coupled to the processor. The processor further can be suitably programmed to identify the origin of the captured nucleic acid based on comparing the sequence of that nucleic acid to sequences stored in a library stored in the computer-readable medium. Optionally, the library stores nucleic acid sequences for a human and for a plurality of pathogens. Optionally, the output indicates the relative number of a pathogen per human cell, where the number of human cells can be defined for example by the number of cells detected by light, electromagnetic, thermal, mass, volume displacement or inferred by nucleic acid, protein, lipid or other chemical component.

In some embodiments, method 20 further includes receiving by the device a second biological sample obtained from the subject, the second biological sample being different from the first biological sample. The method further can include capturing by the device a second plurality of nucleic acids of the first set that are present in the second biological sample. The method further can include, for each of the captured nucleic acids of the second plurality, quantifying by the device an amount of that captured nucleic acid; sequencing by the device that captured nucleic acid; and, based on the sequence of that captured nucleic acid, identifying by the device an origin of that captured nucleic acid. The outputting by the device further can include an indication of the quantified amount and the identified origin of at least one captured nucleic acid that is present in the second biological sample. For example, in some embodiments, the first set of complementary nucleic acids further captures a second plurality of nucleic acids of the first set that are present in a second biological sample obtained from the subject, the second biological sample being different from the first biological sample. The nucleic acid quantifier further can quantify an amount of each of the captured nucleic acids that is present in the second biological sample. The nucleic acid sequencer further can sequence each of the captured nucleic acids that is present in the second biological sample. The processor further can be suitably programmed so as to identify an origin of each captured nucleic acid based on the sequence of the captured nucleic acid that is present in the second biological sample. The processor further can be suitably programmed so as to cause the display to output the an indication of quantified amount and the identified origin of at least one captured nucleic acid that is present in the second biological sample.

In some embodiments, method 20 further includes outputting by the device an indication of at least one potential diagnosis for the subject and an indication of the likelihood of the at least one based on the quantified amount and the identified origin of at least one captured nucleic acid that is present in the first biological sample. For example, in some embodiments, the processor further is suitably programmed to cause the display to output an indication of at least one potential diagnosis for the subject and an indication of the likelihood of the at least one diagnosis based on the quantified amount and the identified origin of at least one captured nucleic acid that is present in the first biological sample.

It is further noted that suitable aspects of the present devices and methods can be implemented using various types of data processor environments (e.g., using one or more data processors) which execute instructions (e.g., software instructions) to perform operations disclosed herein. Non-limiting examples include implementation on a single general purpose computer or workstation, or on a networked system, or in a client-server configuration, or in an application service provider configuration. For example, suitable aspects of the methods and devices described herein may be implemented using many different types of processing devices by program code comprising program instructions that are executable by the device processing subsystem. The software program instructions may include source code, object code, machine code, or any other stored data that is operable to cause a processing system to perform the methods and operations described herein. Other implementations may also be used, however, such as firmware or even appropriately designed hardware configured to carry out the methods and devices described herein. For example, a computer can be programmed with instructions to perform suitable steps of the flowcharts or exemplary analyses shown in FIGS. 4A-20.

It is further noted that the devices and methods may include data signals conveyed via networks (e.g., local area network, wide area network, internet, combinations thereof, etc.), fiber optic medium, carrier waves, wireless networks, etc. for communication with one or more data processing devices. The data signals can carry any or all of the data disclosed herein that is provided to or from a device.

The devices' and methods' data (e.g., associations, mappings, data input, data output, intermediate data results, final data results, etc.) may be stored and implemented in one or more different types of computer-implemented data stores, such as different types of storage devices and programming constructs (e.g., RAM, ROM, Flash memory, flat files, databases, programming data structures, programming variables, IF-THEN (or similar type) statement constructs, etc.). It is noted that data structures describe formats for use in organizing and storing data in databases, programs, memory, or other computer-readable media for use by a computer program.

The devices and methods may be provided on many different types of computer-readable storage media including computer storage mechanisms (e.g., non-transitory media, such as CD-ROM, diskette, RAM, flash memory, computer's hard drive, etc.) that contain instructions (e.g., software) for use in execution by a processor to perform the methods' operations and implement the devices described herein.

Additionally, the computer components, analysis modules, software modules, functions, data stores and data structures (e.g., databases) described herein may be connected directly or indirectly to each other in order to allow the flow of data needed for their operations. It is also noted that a module or processor includes but is not limited to a unit of code that performs a software operation, and can be implemented for example as a subroutine unit of code, or as a software function unit of code, or as an object (as in an object-oriented paradigm), or as an applet, or in a computer script language, or as another type of computer code. The software components and/or functionality may be located on a single computer or distributed across multiple computers depending upon the situation at hand.

It should be understood that as used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise. Finally, as used in the description herein and throughout the claims that follow, the meanings of “and” and “or” include both the conjunctive and disjunctive and may be used interchangeably unless the context expressly dictates otherwise; the phrase “exclusive or” may be used to indicate situation where only the disjunctive meaning may apply.

Although the disclosure has been described with reference to the disclosed embodiments, those skilled in the art will readily appreciate that the specific examples detailed above are only illustrative of the disclosure. It should be understood that various modifications can be made without departing from the spirit of the disclosure. The appended claims are intended to cover all such changes and modifications that fall within the true spirit and scope of the invention.

What is claimed is:

1. A method executed on a device comprising a high-throughput nucleic acid sequencer and a processor, the method comprising:
(A) receiving a first biological sample obtained from a subject with a physical symptom;
(B) receiving a first symptom-specific cartridge comprising a first set of complementary nucleic acids associated with the physical symptom;
(C) contacting the first symptom-specific cartridge with the first biological sample obtained from the subject;
(D) obtaining one or more captured nucleic acids bound to the first symptom-specific cartridge;
(E) automatically preparing the one or more captured nucleic acids for quantifying and sequencing, wherein preparing comprises labeling the one or more captured nucleic acids;
(F) obtaining, by the high-throughput nucleic acid sequencer, data comprising a sequence of the one or more captured nucleic acids;
(G) receiving continuously, by the processor, the data comprising the sequence of the one or more captured nucleic acids;
(H) determining continuously, by the processor, for each sequence of the one or more captured nucleic acids
(i) a cumulative read count, and
(ii) a nucleic acid identity of the one or more captured nucleic acids, wherein the nucleic acid identity comprises one or more of the following:
(a) an anatomical location of the one or more captured nucleic acids;
(b) a cellular response associated with the one or more captured nucleic acids, or
(c) a cell type of origin of the one or more captured nucleic acids;
(I) determining continuously, by the processor, one or more numerical values based on the cumulative read count and the nucleic acid identity for the sequence of the one or more captured nucleic acids; and
(J) repeating continuously steps (F) to (I) until at least one of the numerical values reaches a pre-defined threshold.

2. The method of claim 1, further comprising removing the first symptom-specific cartridge from the device and receiving by the device a second symptom-specific cartridge comprising a second set of complementary nucleic acids.

3. The method of claim 2, wherein the first set of complementary nucleic acids is different than the second set of complementary nucleic acids.

4. The method of claim 1, further comprising outputting by the device an indication of the quantified amount the read count of each of the captured nucleic acids.

5. The method of claim 1, wherein:
the nucleic acids in the first biological sample obtained from the subject are separated into extracellular nucleic acids and intracellular nucleic acids; and
(F) to (H) are performed separately on the extracellular nucleic acids and on the intracellular nucleic acids.

6. The method of claim 5, further comprising outputting by the device a read count of the extracellular nucleic acids and a read count of the intracellular nucleic acids.

7. The method of claim 1, wherein determining the nucleic acid identity comprises comparing the sequence of the one or more captured nucleic acids to sequences stored in a library stored in a computer-readable medium of the device.

8. The method of claim 7, wherein the library stores nucleic acid sequences for a human and for a plurality of pathogens.

9. The method of claim 8, further comprising outputting continuously a relative number of a pathogen per human cell.

10. The method of claim 1, further comprising:
receiving a second biological sample obtained from the subject with a physical symptom, the second biological sample being different from the first biological sample;
contacting the first symptom-specific cartridge with the second biological sample obtained from the subject; and
performing steps (D) to (J).

11. The method of claim 1, wherein the biological sample comprises a biological fluid.

12. The method of claim 10, wherein the biological fluid is selected from the group consisting of blood, urine, sputum, and cerebrospinal fluid (CSF).

13. The method of claim 1, wherein the high-throughput nucleic acid sequencer comprises a next-generation sequencing (NGS) platform.

14. A system for use in diagnosing a condition based on a symptom experienced by a subject and based on a biological sample obtained from the subject, the biological sample including nucleic acids, the system comprising:
means for quantifying an amount of a first subset of the nucleic acids that are present in the biological sample over a first period of time, the first subset of the nucleic acids having a first origin;
means for quantifying an amount of a second subset of the nucleic acids that are present in the biological sample over the first period of time, the second subset of the nucleic acids having a second origin that is different than the first origin;
means for outputting an indication of the amount of the first subset of the nucleic acids quantified over the first period of time; and
means for outputting an indication of the amount of the second subset of the nucleic acids quantified over the first period of time.

15. The system of claim 14, further comprising:
means for estimating a first likelihood that the subject is suffering from a first condition based on the amount of the first subset of the nucleic acids quantified over the first period of time;
means for estimating a second likelihood that the subject is suffering from a second condition that is different than the first condition based on the amount of the second subset of the nucleic acids quantified over the second period of time; and
means for outputting an indication of the first likelihood and an indication of the second likelihood.

16. The system of claim 15, further comprising:
means for estimating a first trajectory of an amount of the first subset of the nucleic acids over a second period of time based on the amount of the first subset of the nucleic acids quantified over the first period of time;
means for estimating a second trajectory of an amount of the second subset of the nucleic acids over the second period of time based on the amount of the second subset of the nucleic acids quantified over the first period of time; and
means for outputting an indication of the first trajectory and an indication of the second trajectory based on the amount of the second subset of the nucleic acids quantified over the first period of time.

17. A system for performing one or more nucleic acid tests based on one or more symptoms experienced by a patient, the device comprising:
an input module configured to receive respective identifiers of the one or more symptoms experienced by the patient;
a query module configured to submit to a database a query comprising the respective identifiers of each of the one or more symptoms, the database comprising a computer-readable medium storing at least a plurality of symptoms, a nucleic acid sequence associated with each of the symptoms, a potential diagnosis associated with each of the symptoms, a laboratory test or a procedure for each of the symptoms, and inferred data for each of the symptoms, the inferred value comprising a clinical inference based on a result of said laboratory test for the respective symptom;
the query module further being configured to receive from the database a response to the query, the response comprising one or more nucleic acid tests based on the nucleic acid sequences respectively associated with the one or more symptoms identified in the query;
an output module configured to output respective representations of the one or more nucleic acid tests; and
a receptacle configured to receive a cartridge configured to perform at least one of the one or more nucleic acid tests.

18. The system of claim 17, wherein the output module further is configured to output a result of the at least one of the one or more nucleic acid tests, the result comprising a count of RNA or DNA of the subject or of a pathogen in the subject, the RNA or DNA having the nucleic acid sequence associated with at least one of the one or more symptoms identified in the query.

19. The system of claim 17, wherein the response to the query comprises a representation of plurality of nucleic acid tests based on a plurality of nucleic acid sequences respectively associated with the one or more symptoms identified in the query, the cartridge being configured to perform each nucleic acid test of the plurality.

20. The system of claim 17, wherein the receptacle of the device further is configured to receive least one additional cartridge, the at least one additional cartridge being configured to perform at least one other of the nucleic acid tests.


SEQUENCE LISTING (14 sequences)

SEQ ID NO: 1 - DNA, 16 bp, Artificial Sequence
  Description of Artificial Sequence Synthetic oligonucleotide
atatgaggctagggaa

SEQ ID NO: 2 - DNA, 16 bp, Artificial Sequence
  Description of Artificial Sequence Synthetic oligonucleotide
tatactccgatccctt

SEQ ID NO: 3 - DNA, 16 bp, Artificial Sequence
  Description of Artificial Sequence Synthetic oligonucleotide
gtggtatgatgggtgc

SEQ ID NO: 4 - DNA, 14 bp, Artificial Sequence
  Description of Artificial Sequence Synthetic oligonucleotide
aatgggaacggtaa

SEQ ID NO: 5 - DNA, 14 bp, Artificial Sequence
  Description of Artificial Sequence Synthetic oligonucleotide
tctttcaggtcata

SEQ ID NO: 6 - DNA, 22 bp, Artificial Sequence
  Description of Artificial Sequence Synthetic oligonucleotide
aaatatagaatatgtattgcgg

SEQ ID NO: 7 - DNA, 13 bp, Artificial Sequence
  Description of Artificial Sequence Synthetic oligonucleotide
tgactaagtggca

SEQ ID NO: 8 - RNA, 16 nt, Artificial Sequence
  Description of Artificial Sequence Synthetic oligonucleotide
gugguaugaugggugc

SEQ ID NO: 9 - DNA, 14 bp, Artificial Sequence
  Description of Artificial Sequence Synthetic oligonucleotide
gccatgatcatgat

SEQ ID NO: 10 - DNA, 19 bp, Artificial Sequence
  Description of Artificial Sequence Synthetic oligonucleotide
aggattgccatgcatcacg

SEQ ID NO: 11 - DNA, 19 bp, Artificial Sequence
  Description of Artificial Sequence Synthetic oligonucleotide
tgggacgatccgataaacc

SEQ ID NO: 12 - DNA, 19 bp, Artificial Sequence
  Description of Artificial Sequence Synthetic oligonucleotide
acctacacaggtggaccat

SEQ ID NO: 13 - DNA, 19 bp, Artificial Sequence
  Description of Artificial Sequence Synthetic oligonucleotide
gcgattagcggagtggcta

SEQ ID NO: 14 - DNA, 19 bp, Artificial Sequence
  Description of Artificial Sequence Synthetic oligonucleotide
cctactgacatccgtgcca